(19) United States
(12) Patent Application Publication
(10) Pub. No.: US 2025/0375296 A1
(43) Pub. Date: Dec. 11, 2025

(54) Devices and Systems For Fixating Tissue

(71) Applicant: Evalve, Inc., Santa Clara, CA (US)

(72) Inventors: Chad J. Abunassar, Alameda, CA (US); Grant Glaze, San Francisco, CA (US); Clara Park, Santa Clara, CA (US)

(73) Assignee: Evalve, Inc., Santa Clara, CA (US)

(21) Appl. No.: 19/228,977

(22) Filed: Jun. 5, 2025

Related U.S. Application Data
(60) Provisional application No. 63/656,690, filed on Jun. 6, 2024.
(60) Provisional application No. 63/725,141, filed on Nov. 26, 2024.

(51) Int. Cl. A61F 2/24

(52) U.S. Cl. CPC A61F 2/2466

(57) ABSTRACT

An implantable fixation device includes fixation elements and gripping elements for fixating tissue. A stud is coupled to the fixation elements and is configured such that movement of the stud moves the fixation elements within a range of positions. A binding plate is configured to engage the stud to arrest its movement and to disengage the stud to allow its movement. A biasing element engages the binding plate and biases the binding plate into engagement with the stud. A harness with a single foot is engaged to the binding plate is configured such that tensioning the harness moves the binding plate in opposition to the bias of the biasing element so as to release the binding plate from the stud. Releasing tension on the harness returns the binding plate into engagement with the stud and secures the fixation elements in one position within the range of positions.

### CROSS-REFERENCE TO RELATED APPLICATIONS

This application claims the benefit of U.S. Provisional Application No. 63/656,690, filed Jun. 6, 2024, and U.S. Provisional Patent Application No. 63/725,141, filed Nov. 26, 2024, the disclosures of which are hereby incorporated by reference in their entireties.

### BACKGROUND

The cardiac cycle is divided into two phases-diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrhythmias, pulmonary hypertension, stroke, and endocarditis, to name a few.

MVR and TVR can have a variety of etiologies which typically fall into the categories of degenerative (primary) and functional (secondary) regurgitation. Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart. Examples of conditions that can result in functional regurgitation include dilated cardiomyopathy, ischemic heart disease, pulmonary hypertension, and heart failure. Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### BRIEF SUMMARY OF THE DISCLOSURE

One aspect of the disclosure includes an implantable fixation device for securing tissue. The implantable fixation device may include a first fixation element and a second fixation element. The first and second fixation elements may have a first end, a free end opposite the first end, and an engagement surface therebetween for engaging tissue. The free ends may be moveable between a first position and a second position.

The implantable fixation device may also include an actuation mechanism coupled to the first and second fixation elements. The actuation mechanism may have a stud configured to move in an axial direction. Movement of the stud in the axial direction may move the first and second fixation elements between the first and second positions.

The implantable fixation device may further include a lock. The lock may include a wedging element. The wedging element may have a first surface and an opposing second surface. The wedging element may be at least partially disposed about the stud and may be moveable between a first position in which the wedging element engages the stud to arrest its movement in the axial direction, and a second position in which the wedging element is disengaged from the stud allowing the stud to move in the axial direction.

The lock may further include a biasing element. The biasing element may engage the first surface of the wedging element and may be configured to bias the wedging element toward the first position thereof.

The lock may also include a harness. The harness may have a first end portion and a second end portion. The second end portion may have a single foot. The single foot may be moveable to engage the second surface of the wedging element and to move the wedging element against the bias of the biasing element from the first position to the second position of the wedging element.

In some implementations of the harness, the harness may be configured such that pulling the first end portion of the harness in the axial direction may move the binding plate to the second position and may pivot the harness from a first orientation to a second orientation. The second orientation may be 6 degrees or less from the first orientation.

In further implementations of the harness, the first end portion of the harness may have a first straight segment. When the harness is in the first orientation, the first straight segment may be parallel to a longitudinal axis of the stud. The first end portion of the harness may also have a second straight segment connected to the first straight segment and a loop disposed between the first and second straight segments. The loop may define an eyelet configured to receive a lock line. Additionally, the first and second straight segments may be connected to each other by at least three spot welds. The harness may be formed from a wire. The second straight segment of the first end portion may define a terminal end of the wire. The wire may have a diameter of 0.0075 in. to 0.010 in., but preferably 0.008 in.

In additional implementations of the harness, the first end portion of the harness may have a first straight segment. The single foot may be oriented at an acute angle relative to the first straight segment. The acute angle may be 80 to 85 degrees. Additionally, the distal end portion of the harness may include a first straight segment and a second straight segment. The first straight segment may be angled outwardly relative to the first straight segment of the proximal end portion. The second straight segment may be parallel to the first straight segment of the proximal end portion. The single foot may be connected to and extend inwardly from the second straight segment of the distal end portion.

In some implementations of the lock, the lock may include a housing. The stud may extend into the housing, and the biasing element, wedging element, and single foot may be disposed within the housing. The housing may include a finger projecting inwardly therefrom and may be disposed at a first side of the stud. The single foot of the harness may be disposed at a second side of the stud opposite of the first side. Additionally, the wedging element may have a first end disposed adjacent to the finger and a second end disposed adjacent to the single foot. Also, the implantable fixation device may include a coupling member connected to the housing. The coupling member may be configured to releasably connect to a shaft of a delivery system.

In some implementations of the wedging element, the wedging element may be a binding plate. The binding plate may have a first end, a second end, and an opening extending through the first and second surfaces of the binding plate. The stud may be disposed within the opening of the binding plate. The single foot may be disposed adjacent to the second end of the binding plate such that, when the foot engages the binding plate, the foot pivots the second end of the binding plate about the first end of the binding plate.

In some implementations of the biasing element, the biasing element may be a leaf spring. The leaf spring may have a concave surface, a convex surface, a thickness defined between the concave surface and convex surface. A slot may extend through the concave and convex surfaces. The stud may extend through the slot. The thickness of the of the biasing element may be 0.0064 in. The biasing element may also define a width, and the slot of the biasing element may define a slot width. The width of the biasing element may be 0.060 in. to 0.062 in., and the slot width may be 0.029 in. to 0.031 in.

In some implementations of the actuation mechanism, the actuation mechanism may include first and second legs respectively pivotably connected to the first and second fixation elements. The actuation mechanism may also include a base pivotably connected to the first and second legs. The stud may be connected to and extend from the base.

In some implementations of the fixation device, the fixation device may further include first and second gripping elements. The first and second gripping elements may be respectively disposed opposite the first and second fixation elements and may be moveable relative thereto for capturing tissue therebetween. When tissue is captured between the first and second fixation elements and respective first and second gripping elements, the first and second fixation elements and the lock may define a threshold locking angle. The threshold locking angle may be 30 degrees or less.

Another aspect of the disclosure includes an interventional system for fixating tissue. The interventional system may include a delivery system. The delivery system may include a delivery catheter handle. The delivery catheter handle may have a lock control assembly. The delivery system may also include a delivery catheter. The delivery catheter may have a first end coupled to the delivery catheter handle and a second end remote from the first end. The delivery system may further include a lock line extending from the lock control assembly and through the delivery catheter.

The interventional system may further include a fixation device. The fixation device may be releasably coupled to the second end of the delivery catheter. The fixation device may include a first fixation element and a second fixation element. The first and second fixation elements may have a first end, a free end opposite the first end, and an engagement surface therebetween for engaging tissue. The free ends may be moveable between a first position and a second position.

The implantable fixation device of the interventional system may also include an actuation mechanism coupled to the first and second fixation elements. The actuation mechanism may have a stud configured to move in an axial direction. Movement of the stud in the axial direction may move the first and second fixation elements between the first and second positions.

The implantable fixation device of the interventional system may further include a lock. The lock may include a wedging element. The wedging element may have a first surface and an opposing second surface. The wedging element may be at least partially disposed about the stud and may be moveable between a first position in which the wedging element engages the stud to arrest its movement in the axial direction, and a second position in which the wedging element is disengaged from the stud allowing the stud to move in the axial direction.

The lock may further include a biasing element that engages the first surface of the wedging element. The biasing element may be configured to bias the wedging element toward the first position thereof.

The lock may also include a single-sided harness. The single-sided harness may have a first end portion and a second end portion. The first end portion may define an opening configured to receive the lock line. The second end portion may have a foot. The foot may be moveable to engage the second surface of the wedging element so as to move the wedging element against the bias of the biasing element from the first position to the second position of the wedging element.

In some implementations of the interventional device, the second end of the delivery catheter may define a longitudinal axis. The lock line may extend through the opening of the first end portion of the single-sided harness at a first side of the longitudinal axis. The foot of the single-sided harness may be disposed an opposite second side of the longitudinal axis.

In further implementations of the interventional system, the lock control assembly may include a lock knob and a spool coupled to the lock knob such that rotating the lock knob rotates the spool about a rotational axis. The lock line may extend at least partially about the spool such that rotating the spool about the rotational axis in a first rotational direction tensions the lock line and rotating the spool about the rotational axis in a second rotational direction releases tension on the lock line. The rotational axis may be perpendicular to the lock line and the tension thereof.

In some implementations of the spool, the spool may have a pair of opposing flanges and a core extending therebetween. The lock line may at least partially extend about the core. The core may have a diameter of 0.445 in. The lock knob may be rotatable in the first rotational direction between a first position and a second position. The spool may be configured to generate a tension force on the lock line of 1.25 lb to 6 lbf when the lock knob is rotated between the first and second position.

In further implementation of the lock control assembly, the lock control assembly may include a pin, a first detent corresponding the first position of the lock knob, and a second detent corresponding to the second position of the lock knob. The pin may be engageable with the first and second detents to secure the lock knob in the respective first and second positions thereof. The lock line may have a first end portion and a second end portion. The first end portion of the lock line may be fixedly secured to the lock knob, and the second end portion may be releasably secured to the lock knob. The lock knob may be releasable from the delivery catheter handle. Releasing the lock knob from the handle may allow the second end portion of the lock line to be pulled through the opening of the single-sided harness.

In some implementations of the delivery catheter handle, the delivery catheter handle may include a positioner assembly and an actuator rod connected to the positioning assembly. The actuator rod may extend through the second end of the delivery catheter and into releasable engagement with the stud of the fixation device. The actuator rod may be axially moveable upon actuation of the positioner assembly.

In further implementations of the interventional system, the second end of the delivery catheter may define a periphery, and the proximal portion of the harness may define a runout relative to the periphery of the second end of the delivery catheter upon tensioning of the lock line. The runout may be no greater than 0.011 in. to 0.018 in. The proximal portion of the single-sided harness may include a loop defining the opening. The loop may define an outer dimension of 0.037 in. to 0.041 in. The loop may define an inner dimension of 0.015 in. to 0.025 in. Additionally, the single-sided harness may be formed from a wire having a diameter of 0.0075 in. to 0.008 in.

A further aspect of the disclosure includes a method of fixating tissue via a transcatheter approach. The method may include grasping a first tissue between a first fixation element and a first gripping element of a fixation device. The method may also include grasping a second tissue between a second fixation element and a second gripping element of the fixation device. The method may further include rotating the first and second fixation elements to a threshold locking angle. The threshold locking angle may be defined between the first and second fixation elements and may be 30 degrees or less. The method may also include locking the first and second fixation elements at the threshold angle by disengaging a lock of the fixation device.

Another aspect of the disclosure includes a method of fixating tissue via a transcatheter approach. The method may include grasping a first tissue between a first fixation element and a first gripping element of a fixation device. The method may also include grasping a second tissue between a second fixation element and a second gripping element of the fixation device. The method may further include unlocking the first and second fixation elements by axially translating and pivoting a release harness of a lock of the fixation device from a first orientation to a second orientation. The second orientation may be no greater than 6 degrees from the first orientation. The method may also include rotating the first and second fixation elements between a first position and a second position.

A further aspect of the disclosure includes a method of fixating tissue via a transcatheter approach. The method may include advancing a fixation device to a first tissue and a second tissue via a delivery catheter of a delivery system. The method may also include grasping the first tissue between a first fixation element and a first gripping element of the fixation device. The method may further include grasping the second tissue between a second fixation element and a second gripping element of the fixation device. The method may also include unlocking the first and second fixation elements by tensioning a lock line of the delivery system such that a first end portion of a release harness of a lock of the fixation device advances outwardly relative to a periphery of a distal end of the delivery catheter no more than a distance therefrom. The distance may be selected from a range including 0.011 in. to 0.018 in. The method may further include rotating the first and second fixation elements between a first position and a second position.

In one implementation of the method, the rotating step may move the first fixation element toward the first end portion of the release harness.

In another implementation of the method, the unlocking step may include engaging a foot of a second end portion of the release harness with a wedging element of the lock and disengaging the wedging element from a stud of the fixation device. The unlocking step may also include rotating a spool of the delivery system in a first rotational direction about a rotational axis. The lock line may extend at least partially about the spool. Furthermore, the unlocking step may include tensioning the lock line from a first side of a longitudinal axis of distal end of the delivery catheter and moving a foot of the release harness into engagement with a wedging element of the lock at a second side of the longitudinal axis of the distal end of the delivery catheter.

The method may include locking the first and second fixation elements. Locking may include locking the first and second elements at an angle between the first and second positions thereof. Further, locking may be performed by releasing tension on the lock line such that the first end portion of the release harness moves to a position bounded within a periphery of the distal end of the delivery catheter. Additionally, locking may be performed by rotating the spool in a second rotational direction about the rotational axis to release tension on the lock line.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2A is a schematic view of a mitral valve during normal coaptation.

FIG. 2B is a schematic view of a mitral valve during regurgitate coaptation.

FIGS. 3A and 3B are schematic views of a fixation device according to an embodiment of the present disclosure grasping leaflets of a mitral valve.

FIG. 4A is a perspective view of a fixation device according to another embodiment of the present disclosure.

FIG. 4B is a perspective view of the fixation device of FIG. 4A including a covering.

FIG. 5A is a perspective view of a gripping device of the of the fixation device of FIG. 4A according to an embodiment of the present disclosure.

FIG. 5B is an elevational view of the gripping device of FIG. 5A.

FIG. 6A is a perspective view of a gripping device according to another embodiment of the present disclosure.

FIG. 6B is a partial schematic view of the gripping device of FIG. 6A coupled to a distal element of the fixation device of FIG. 4A.

FIG. 6C is a partial schematic view of a gripping device according to an alternative embodiment of the present disclosure coupled to a distal element according to an alternative embodiment of the present disclosure.

FIG. 7A is an elevational view of a coupling system according to an embodiment of the present disclosure for coupling the fixation device of FIG. 4A and a delivery system.

FIGS. 7B and 7C are schematic views of the coupling system of FIG. 7A in respective first and second configurations.

FIGS. 8A and 8B are schematic cross-sectional views of a coupling system according to another embodiment of the present disclosure for coupling a fixation device, such as the fixation device of FIG. 4A, and a delivery system.

FIGS. 9A-9B, 10A-10B, 11A-11B, 12A-12B and 13A-13C illustrate the fixation device of FIG. 4A in various possible positions during introduction and placement of the device within a mammalian body to perform a therapeutic procedure.

FIG. 14 is a perspective view of the fixation device of FIG. 4A including a lock according to an embodiment of the present disclosure and illustrating a plurality of proximal element lines and a lock line coupled to the fixation device.

FIG. 15 is an elevational view of the lock and proximal elements of the fixation device of FIG. 14 and illustrating a lock line and single proximal element line respectively coupled thereto.

FIG. 16 is a schematic view of the fixation device of FIG. 4A coupled to a delivery system and illustrating a plurality of proximal element lines coupled to a shaft of the delivery system.

FIGS. 17A and 17B are partial enlarged views of a distal end portion of the delivery system shaft of FIG. 16 according to an embodiment of the present disclosure.

FIG. 17C is a cross-sectional view of the delivery system shaft taken along line C-C of FIG. 17B.

FIG. 17D is a partial perspective view of a distal end portion of one of the proximal element lines of FIG. 16 including a catch element according to an embodiment of the present disclosure.

FIG. 17E is a partial elevational view of the delivery system shaft of FIG. 17A having holes configured to receive the catch element of FIG. 17D.

FIG. 17F is a partial elevational view of the delivery system shaft of FIG. 17A and an actuator rod disposed therein intersecting the holes of the delivery system shaft.

FIG. 17G is a partial elevational view of a distal end portion of one of the proximal element lines of FIG. 16 including a catch element according to another embodiment of the present disclosure.

FIG. 18A is an enlarged cross-sectional view of the lock of FIG. 14 taken along a midline thereof and in an unlocked configuration.

FIG. 18B is an enlarged elevational view of the lock of FIG. 14 and in a locked configuration.

FIG. 18C is a perspective view of a release harness of the lock of FIG. 14.

FIG. 19A is an elevational view of a lock of the fixation device of FIG. 4A according to another embodiment of the present disclosure.

FIG. 19B is a transparent perspective view of a binding plate of the lock of FIG. 19A.

FIG. 19C is an enlarged elevational view of the lock of FIG. 19A.

FIG. 20A is a perspective view of an exemplary fixation device including the lock of FIG. 19A, the lock being in an unlocked configuration.

FIG. 20B is an enhanced partial view of the lock of FIG. 20A.

FIG. 20C is a perspective view of the fixation device of FIG. 20A with the lock thereof being in a locked configuration.

FIG. 21A is a perspective view of an exemplary fixation device including a lock according to a further embodiment of the present disclosure.

FIG. 21B is an enhanced view of the lock of FIG. 21A.

FIG. 22A is a perspective view of a biasing element of the lock of FIG. 21A.

FIG. 22B top view of the biasing element of FIG. 22A.

FIG. 22C is a bar graph comparing the flattening of a biasing element of the lock of FIG. 19A and examples of the biasing element of FIG. 22A when subjected to a cyclical load.

FIG. 22D is a table tabulating a finite element analysis comparing the compression force, locked force, and locked strain of a biasing element of the lock of FIG. 19A and examples of the biasing element of FIG. 22A.

FIG. 23A is a perspective view of a release harness of the lock of FIG. 21A.

FIG. 23B is side elevational view of the release harness of FIG. 23A.

FIG. 23C is a front elevational view of the release harness of FIG. 23A.

FIG. 23D is an enhanced partial view of the release harness of FIG. 23A.

FIG. 24A is perspective view of the lock of FIG. 21A in an unlocked configuration.

FIG. 24B is an enhanced view of the lock of FIG. 24A.

FIG. 25A line graph comparing the unlocking force between the lock of FIG. 19A and the lock of FIG. 21A when subjected to cyclical loading.

FIG. 25B is a bar graph comparing the unlocking force between the lock of FIG. 19A and the lock of FIG. 21A with and without captured leaflets and at various angles of closure of a fixation device.

FIG. 26A is a top view of a binding plate according to another embodiment of the present disclosure.

FIG. 26B is a partial perspective view illustrating engagement between the release harness of FIG. 23A and the binding plate of FIG. 26A.

FIG. 26C is a partial view showing a force vector produced by the engagement of the release harness of FIG. 23A and the binding plate of FIG. 26A.

FIG. 27A is a partial view of an exemplary fixation device including an exemplary lock having the release harness of FIG. 23A in an alternative arrangement and in a locked configuration.

FIG. 27B is a partial view of the lock of FIG. 27A in an unlocked configuration.

FIG. 27C is an enhanced view of a loop of the release harness of FIG. 27A and a lock line engaged therewith.

FIG. 28 is a partial perspective view of a distal end of a release harness according to another embodiment of the present disclosure showing a single bump extending from a foot thereof.

FIG. 29 is a partial perspective view of a distal end of a release harness according to a further embodiment of the present disclosure showing a plurality of bumps extending from a foot thereof.

FIG. 30 is a partial perspective view of a distal end of a release harness according to yet another embodiment of the present disclosure showing a reinforcement proximate the foot thereof.

FIG. 31A is a top view of a binding plate according to a further embodiment of the present disclosure.

FIG. 31B is a side elevational view of the binding plate of FIG. 31A.

FIG. 31C is an end view of the binding plate of FIG. 31A.

FIG. 31D is a partial perspective view illustrating engagement between the release harness of FIG. 23A and the binding plate of FIG. 31A.

FIG. 31E is a partial view showing a force vector produced by the engagement of the release harness of FIG. 23A and the binding plate of FIG. 31A.

FIG. 31F is a table comparing opening force measurements between fixation devices having the binding plate of FIG. 26A and fixation devices having the binding plate of FIG. 31A.

FIG. 31G is a graph visually representing the data from FIG. 31F.

FIG. 31H is a table tabulating comparing unlock force measurements between the fixation devices having the binding plate of FIG. 26A and fixation devices having the binding plate of FIG. 31A

FIG. 31I is a graph visually representing the data from FIG. 31H.

FIG. 32 is a top view of a binding plate according to another embodiment of the present disclosure.

FIG. 33 is a perspective view of a binding plate according to a further embodiment of the present disclosure.

FIG. 34A is a top view of a binding plate according to yet another embodiment of the present disclosure.

FIG. 34B is a perspective view of the binding plate of FIG. 34A interacting with a lock housing according to another example of the present disclosure.

FIG. 34C is a partially transparent front view showing the interaction between the binding plate of FIG. 34A and the lock housing of FIG. 34B.

FIG. 35A is a bottom view of a binding plate according to still another embodiment of the present disclosure.

FIG. 35B is a side elevational view of the binding plate of FIG. 35A.

FIG. 36 is a top view of a binding plate according to another embodiment of the present disclosure.

FIG. 37 is a top view of a binding plate according to a further embodiment of the present disclosure.

FIG. 38 is a top view of a binding plate according to yet another embodiment of the present disclosure.

FIG. 39 is a top view of a binding plate according to a still further embodiment of the present disclosure.

FIG. 40 is a top view of a binding plate according to an even further embodiment of the present disclosure.

FIG. 41 is a partial perspective view of an interventional system according to an embodiment of the present disclosure and including an exemplary delivery system, an exemplary steerable guide system, and an exemplary stabilizer.

FIG. 42A is a partial perspective view of the delivery system of FIG. 41.

FIG. 42B is a partial cutaway view of the delivery system of FIG. 41 and the fixation device of FIG. 4A coupled thereto.

FIG. 43A is a cross-sectional view of a positioner assembly of the delivery system of FIG. 41.

FIG. 43B is a partial perspective view of the positioner assembly of FIG. 43A including a rotation control system according to an embodiment of the present disclosure and in an example of a first configuration.

FIGS. 43C and 43D are partial perspective views of the positioner assembly and rotation control system of FIG. 43B with the rotation control system in an example of a second configuration.

FIG. 44A is a partial perspective view of a gripper control assembly and a fluid management assembly of the delivery system of FIG. 41 and each according to an embodiment of the present disclosure.

FIG. 44B is a partial elevational view of the gripper control assembly of FIG. 44A.

FIG. 44C is a partial cross-sectional view of the gripper control assembly of FIG. 44A taken along a midline thereof.

FIG. 45A is a partial transparent perspective view of a lock control assembly of the delivery system of FIG. 41 according to an embodiment of the present disclosure.

FIG. 45B is another partial transparent perspective view of the lock control assembly of FIG. 45A.

FIG. 45C is an exploded view of the lock control assembly of FIG. 45A.

FIGS. 46A and 46B are perspective views of a lock handle shaft of the lock control assembly of FIG. 45A in an example of an unlocked configuration.

FIGS. 46C and 46D are perspective views of a lock handle shaft of the lock control assembly of FIG. 45A in an example of an unlocked configuration.

FIG. 46E is a perspective view of a post of lever of lock handle shaft of the lock control assembly of FIG. 45A.

FIG. 47A is an elevational view of a spool of the lock control assembly of FIG. 45A and according to one example.

FIG. 47B is a cross-sectional view of the spool of FIG. 47A taken along a midline thereof.

FIG. 48A is an elevational view of a spool of the lock control assembly of FIG. 45A and according to another example.

FIG. 48B is another elevational view of the spool of FIG. 48A.

FIG. 49A is a partial transparent perspective view of the lock control assembly of FIG. 45A with a lock knob thereof in an example of a lock configuration.

FIG. 49B is a partial transparent perspective view of the lock control assembly of FIG. 45A with a lock knob thereof in an example of an unlocked configuration.

FIG. 49C is a partial transparent perspective view of the lock control assembly of FIG. 45A with a lock knob thereof in an example of a third configuration.

FIGS. 49D and 49E are partial transparent perspective views of the lock control assembly of FIG. 45A with a release slider thereof in an example of a release position.

FIG. 49F is a partial transparent perspective view of the lock control assembly of FIG. 45A including lock knob thereof being removed.

FIG. 49G is a perspective view of the lock control assembly of FIG. 45A partially disassembled.

FIG. 50 is a schematic view of the lock of FIG. 21A and the spool of FIG. 48A in operation.

FIG. 51 is a line chart comparing the maximum lock line force of the spool of FIG. 32A and the spool of FIG. 48A.

FIG. 52A is a cross-sectional view of a delivery catheter fastening assembly of the delivery system of FIG. 41 according to an embodiment of the present disclosure.

FIG. 52B illustrates the delivery catheter fastening system of FIG. 52A being rotated in first and second directions.

FIGS. 52C and 52D illustrate the delivery system of FIG. 41 translating between first and second positions along a shaft of the delivery catheter fastening assembly of FIG. 52A.

FIG. 53 is a partial elevational view of the interventional system of FIG. 41 including a proximal attachment assembly thereof.

FIG. 54A is a perspective view of the proximal attachment assembly of FIG. 53.

FIGS. 54B and 54C are elevational views of the proximal attachment assembly of FIG. 54A in an example of a first configuration and an example of a second configuration, respectively.

FIGS. 54D and 54E are elevational views of the proximal attachment assembly of FIG. 54A in the first configuration and the second configuration, respectively.

FIG. 55 is partial perspective view of the interventional system of FIG. 41 including an exemplary distal attachment assembly thereof.

FIG. 56A illustrates the distal attachment connected to an outer guide catheter handle of the interventional system shown in a cutaway view.

FIG. 56B is a partial cross-sectional view of the distal attachment assembly of FIG. 56A taken along a midline thereof.

FIG. 57A is a perspective view of the stabilizer of FIG. 41.

FIG. 57B is a partial perspective view of a distal attachment of the stabilizer of FIG. 57A.

FIG. 57C is a partial perspective view of a proximal attachment of the stabilizer of FIG. 57A.

FIGS. 58A and 58B illustrate the distal attachment assembly of FIG. 56A being attached to the distal attachment FIG. 57A.

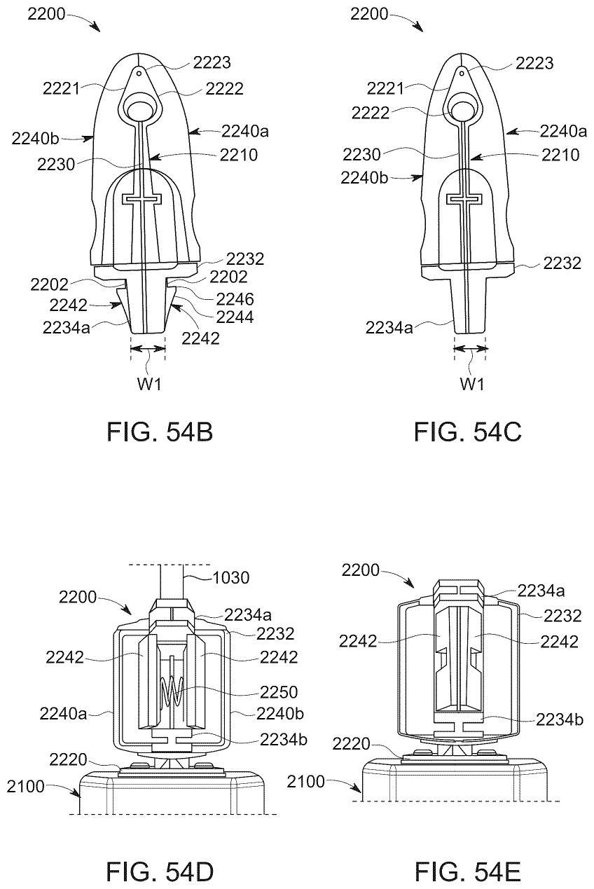

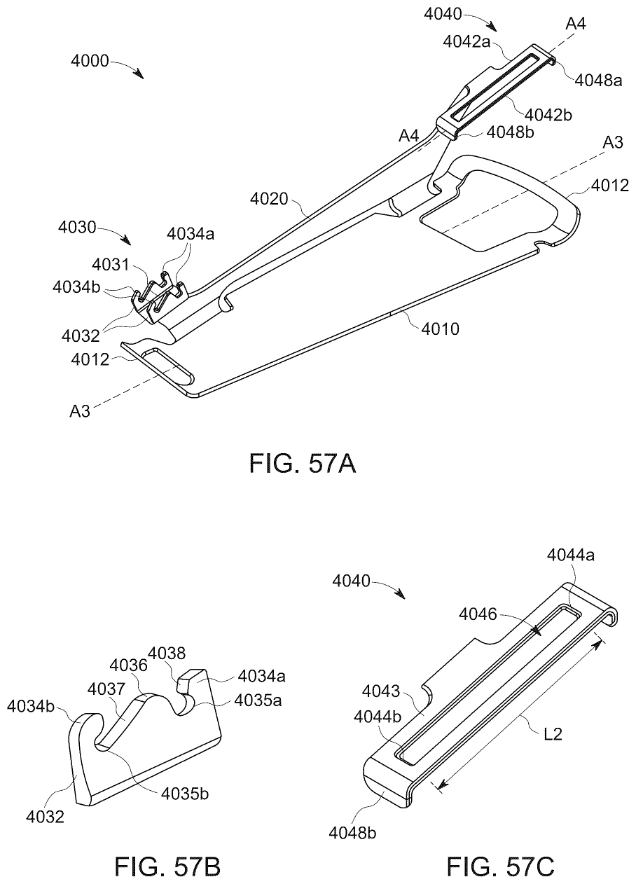

FIG. 57B is a partial perspective view of a distal attachment of the stabilizer of FIGS. 59A and 59B illustrate the proximal attachment assembly of FIG. 54A attached to the proximal attachment of FIG. 57A.

FIGS. 60A and 60B illustrate the proximal attachment assembly of FIG. 54A being moved relative to the proximal attachment of the stabilizer of FIG. 57A.

FIGS. 61A and 61B illustrate an example of manipulation of the fixation device during a procedure using the interventional system and stabilizer of FIG. 41.

### DETAILED DESCRIPTION

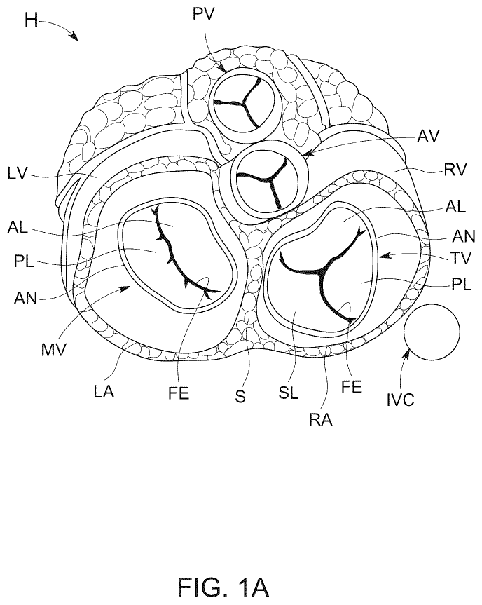

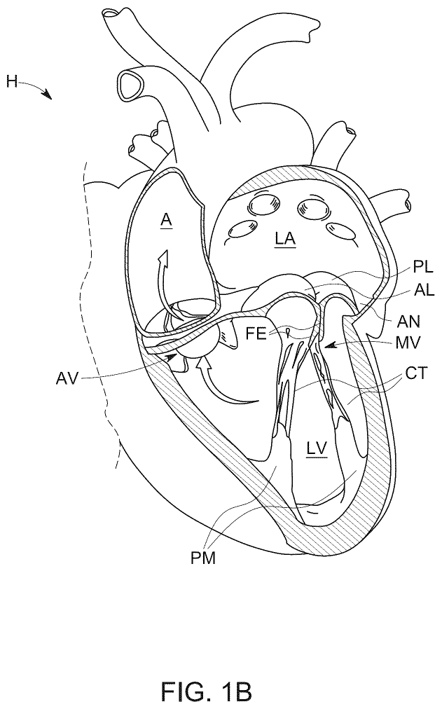

The valves of a normal heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, and the tricuspid valve TV separates the right atrium RA and the right ventricle RV. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV typically has three leaflets referred to as the anterior leaflet AL, the posterior leaflet PL, and the septal leaflet SL. However, studies have shown that, although the TV is typically composed of three leaflets of unequal size, in many cases, two or more than three leaflets may be present as anatomic variants in healthy subjects. Thus, reference herein to the tricuspid valve TV should be understood to refer to the atrioventricular valve located between the right atrium RA and right ventricle RV regardless of the number of leaflets be it two, three, or more than three leaflets. However, exemplary embodiments discussed herein refer to the usual anatomic structure of the tricuspid valve TV that includes three leaflets.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

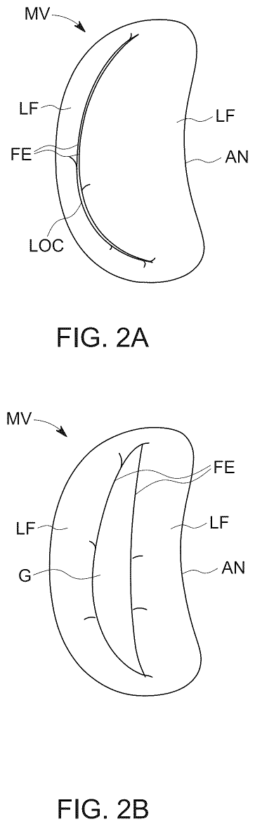

The mitral valve MV depicted in FIGS. 1B and 2A illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and left atrium LA respectively sealed off by the mitral valve MV and tricuspid valve TV, blood in the left ventricle LV can only flow through the aortic valve AV to the body, and blood in the right ventricle RV can only flow through the pulmonary valve PV to the lungs.

A number of structural defects in the heart H can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2B illustrates valvular insufficiency of an atrioventricular valve resulting in regurgitation. In the depicted example, an enlargement of the heart H may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet via the chordae CT. While an adjacent leaflet LF may maintain a normal profile, the prolapsing leaflet LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

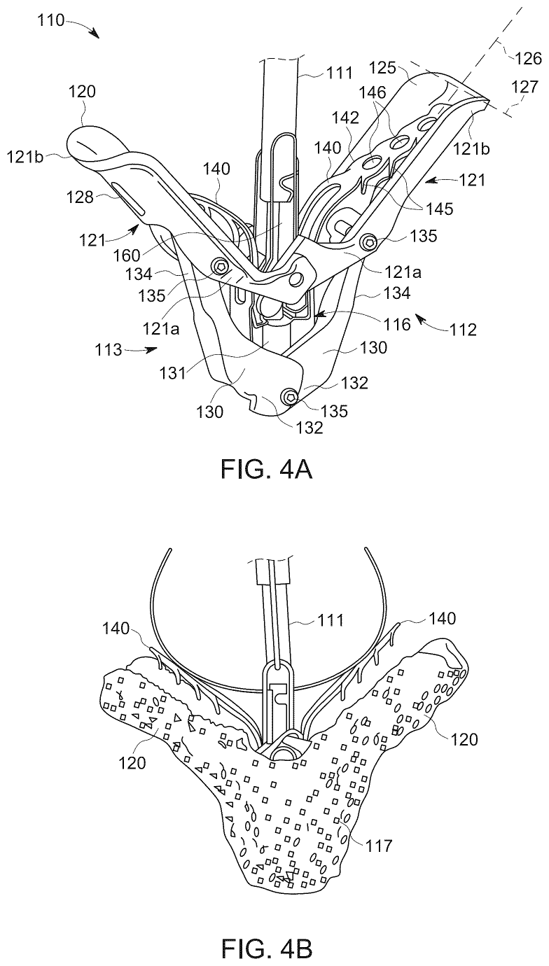

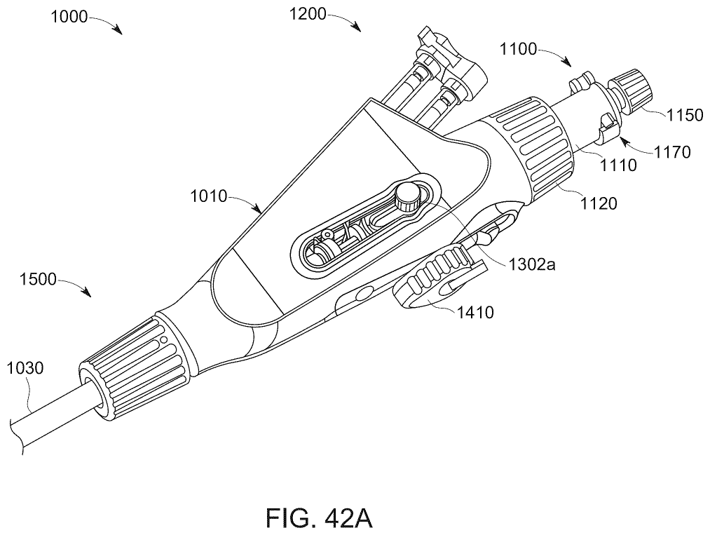

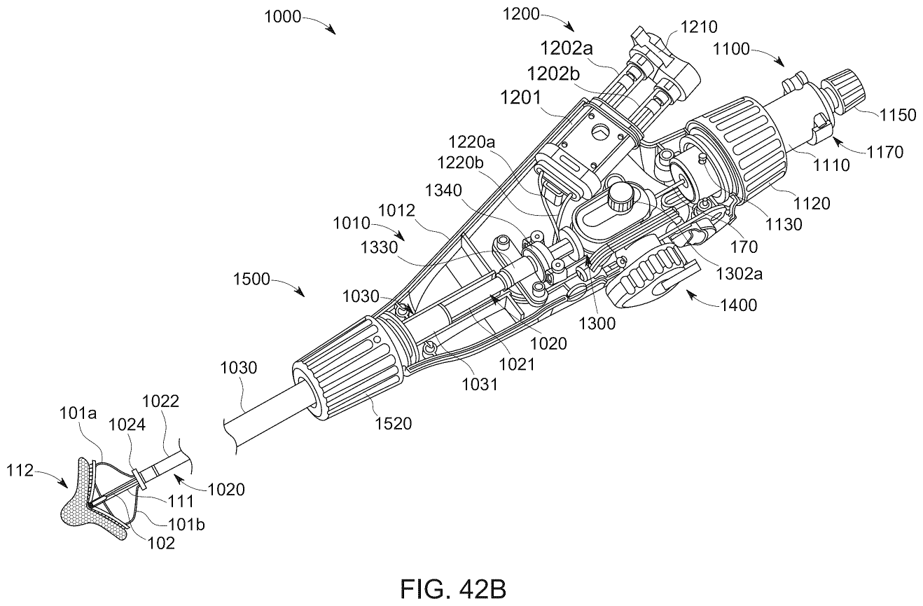

The present disclosure describes exemplary systems, devices, and methods for percutaneously repairing a valve to treat cardiac valve regurgitation, particularly MVR and TVR. For example, an interventional system 3, according to an embodiment of the present disclosure, may include a fixation device 112 (see e.g., FIG. 4A), a delivery system 1000 (see e.g., FIG. 42B) for delivery to and deployment of a fixation device (e.g., fixation device 112) within a heart valve, a steerable guide system 5 (see e.g., FIG. 41) for providing a conduit to guide delivery system 1000 and fixation device 112 to the heart valve, and a stabilizer 4000 (see e.g., FIG. 57A) for stabilizing and supporting the use of delivery system 1000 and the steerable guide system 5. The delivery system 1000 and steerable guide system 5 are collectively referred to herein as a multi-catheter system.

When referring to such disclosed systems, devices, and methods, the term “proximal” (P) shall mean closer to the user or in a direction toward a device to be manipulated by the user outside the patient's body (e.g., a delivery handle 1010 of delivery system 1000), and the term “distal” (D) shall mean more distant from the user or in a direction toward a device that is positioned at the treatment site within the patient's body (e.g., fixation device 112). With respect to the mitral valve and tricuspid valve, “proximal” shall refer to the atrial or upstream side of the valve leaflets, and “distal” shall refer to the ventricular or downstream side of the valve leaflets.

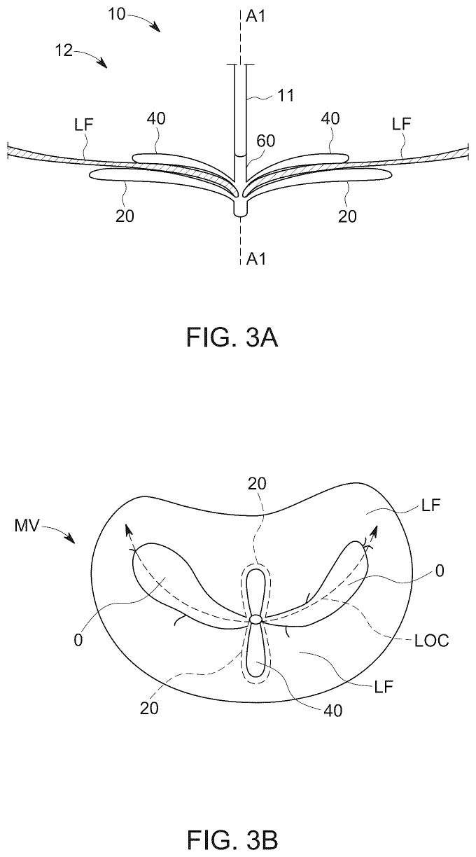

FIGS. 3A and 3B depict a fixation device 12, according to an embodiment of the present disclosure, grasping leaflets LF of an atrioventricular valve, which is illustrated as a mitral valve MV. Fixation device 12 may be releasably coupled to a distal end of a shaft 11 of a delivery system (e.g., delivery system 100) to form an interventional tool 10. Fixation device 12 may include distal elements 20 (also referred to herein as fixation elements) and proximal elements 40 (also referred to herein as gripping elements). Distal and proximal elements 20, 40 may be moveable relative to each other and may protrude radially outward relative to a longitudinal axis A1 of fixation device 12. As shown in FIG. 3A, fixation device 12 may be positionable on opposite sides of adjacent leaflets LF of the valve so as to capture or retain the leaflets LF therebetween. In this regard, proximal elements 40 may be positioned at a proximal side of the valve leaflets LF, and distal elements 20 may be positioned on a distal side of the valve leaflets LF. Proximal elements 40 may be made from cobalt chromium, nitinol, or stainless steel, for example, and distal elements 20 may be made from cobalt chromium or stainless steel, for example.

Fixation device 12 may be releasably coupled to shaft 11 such that it can be detached and left behind as an implant to hold the leaflets LF together in the coapted position. In this regard, fixation device 12 may be delivered to a target valve percutaneously using any one of a number of different approaches, such as via a transfemoral, a transapical, or a transjugular approach, for example. Thus, in one example of treating MVR, fixation device 12 may be delivered to the deficient mitral valve MV using a transfemoral approach in which fixation device 12 is guided through the inferior vena cava IVC (see FIG. 1A), across the interatrial septum S, and into left atrium LA where fixation device 12 is advanced into the mitral valve MV. Also, in one example of treating TVR, fixation device 12 may be guided transfemorally through the inferior vena cava IVC to the right atrium RA where fixation device 12 is advanced to a desired position within the tricuspid valve TV.

FIG. 3B is an atrial-side view of fixation device 12 in one example of a desired orientation in relation to adjacent leaflets LF of an atrioventricular valve, such as the depicted mitral valve MV. The distal and proximal elements 20, 40 are positioned to be substantially perpendicular to the line of coaptation LOC. Thus, in the case of a mitral valve MV, fixation device 12 may be oriented perpendicular (±5 degrees) to a line of coaptation LOC between the posterior leaflet PL and anterior leaflet AL, and in the case of a tricuspid valve TV, fixation device 12 may be positioned perpendicular (±5 degrees) to a line of coaptation between the septal leaflet SL and the anterior leaflet AL, the septal leaflet SL and the posterior leaflet PL, or the anterior leaflet AL and the posterior leaflet PL, for example. Fixation device 12 may be moved roughly along the line of coaptation LOC to the location of regurgitation. The leaflets LF may be held in place so that, during diastole, the leaflets LF remain in position between elements 20, 40 surrounded by openings O (also referred to herein as orifices) which result from the diastolic pressure gradient. Advantageously, leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, fixation device 112 may be repositioned. This may be repeated until an optimal result is produced wherein the leaflets LF are held in place.

FIGS. 4A-19C depict a fixation device 112 according to another embodiment of the present disclosure. Fixation device 112 may generally include a pair of distal elements 120, a pair of proximal elements 140, a coupling member 160, an actuation mechanism 113, and a stud 131. Distal elements 120 may include elongate arms 121 in which each arm has a proximal end portion 121a, which may be rotatably connected to the coupling member 160, and a free end 121b, as best shown in FIG. 4A. Free ends 121b may each have a rounded shape to minimize interference with and trauma to surrounding tissue structures according to one example. In one example, each free end 121b defines a curvature extending about two axes 126, 127. The first axis 126 may be a longitudinal axis of each respective arm 121. Additionally, arms 121 may each include an engagement surface 125 that may also be curved about first axis 126 and may extend at least partially along a length of arm 121 to the free end 121b. Thus, in some examples, engagement surfaces 125 may each have a cupped or concave shape which may maximize contact area engagement with tissue and may assist in grasping and holding valve leaflets. Such cupped or concave shape may further allow arms 121 to nest around shaft 111 of interventional tool 110 while in the closed position to minimize the profile of fixation device 112. Thus, arms 121 may be at least partially cupped or curved inwardly about their longitudinal axes 126 which may form a concavity extending along axis 126 which may nest proximal elements 140 when in a lowered position thereof. The second axis 127 about which each free end 121b may be curved may extend perpendicular to first axis 126, as is also shown in FIG. 4A. The curvature about this second axis 127 may be a reverse curvature located at the most distal portion of free ends 121b. In addition to the dual curvature, free ends 121b may flare outwardly at their respective longitudinal edges. It is believed that both the reverse curvature and flare help create an atraumatic configuration that minimizes trauma to the tissue engaged therewith.

In the nonlimiting embodiment depicted, a transverse width across engagement surfaces 125 (which is in the direction of second axis 127 and determines the width of tissue engaged) may be at least about 2 mm, 3-10 mm in some examples, and about 4-6 mm in some examples. In some embodiments, a wider engagement may be desired wherein the engagement surfaces 125 are larger, for example about 2 cm, or multiple fixation devices 112 may be used adjacent to each other. Arms 121 may also have a length of about 6-12 mm (defined along first axis 126), and engagement surfaces 125 may be configured to engage a length of tissue of about 4-10 mm along the longitudinal axis 126 of arms 121 according to some examples. Also, as shown in the illustrated example, each arm 121 may include a plurality of openings 128 to enhance grip and to promote tissue ingrowth following implantation.

In one example, actuation mechanism 113 may include two link members or legs 130. Legs 130 may be comprised of a rigid or semi-rigid metal or polymer such as Elgiloy®, cobalt chromium or stainless steel, however any suitable material may be used. Each leg 130 may have a first end 132, which may be rotatably joined with one of the distal elements 120 at a riveted joint 135, and a second end 134, which may be rotatably joined with stud 131, as shown in FIG. 4A. Although the depicted embodiment shows both legs 130 pinned to stud 131 by a single rivet 135, it is also contemplated that each leg 130 may be individually attached to the stud 131 by a separate rivet, pin or the like. In other embodiments of actuation mechanism 113, actuation mechanism 113 may include a base 139, and second ends 134 of legs 130 may be rotatably joined with base 169, such as by one or more riveted joints 135, as best shown in FIG. 10B. An actuator rod 170 of delivery system 1000 may be joinable with actuation mechanism 113 directly, such as via direct connection with base 139, or indirectly, such as via connection with stud 131, which itself may extend from base 139. In either of these embodiments, actuator rod 170 may be axially extendable and retractable in a proximal-distal direction to actuate actuation mechanism 113 and consequently rotate distal elements 120 between open, closed, and inverted positions, which are described further below. Additionally, coupling member 160, stud 131, and/or base 169 may comprise a center portion or center body of fixation device, for example.

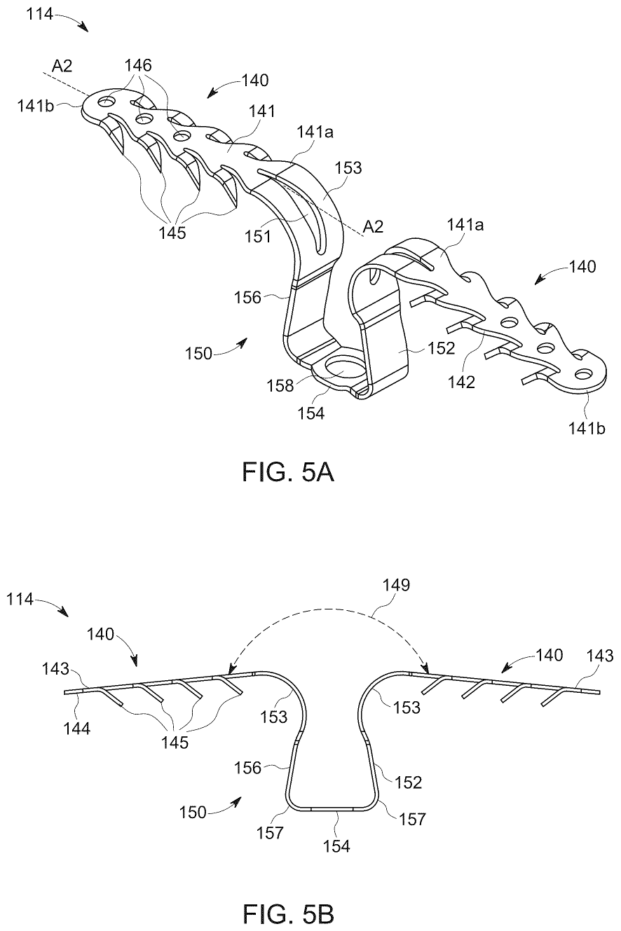

Proximal elements 140 may, in some examples, be flexible, resilient, and cantilevered from a center of fixation device 112. For example, FIGS. 5A and 5B depict a gripping device 114 according to an embodiment of the present disclosure that may generally include a pair of proximal elements 140, a base section 150, and a pair of arm bend features 153 partitioning proximal elements 140 from base section 150.

Proximal elements 140 may be in the form of elongate arms 141 that each extend along a longitudinal axis A2 from a first end portion or fixed end 141a to a second end portion or free end 141b, as shown in FIG. 5A. Each proximal element 140 may also have opposed side edges 142 that define a width transverse to the longitudinal axis A2. Such width may be less than the width of a corresponding distal element 120 such that proximal element 140 may be recessed within the concavity formed by engagement surface 125 of distal element 120 when proximal element 140 is moved into a lowered position, as described in more detail below.

Proximal elements 140 may also each have a first side or proximal side 143 and a second side or distal side 144. In one example, proximal elements 140 may include a plurality of openings 146 that may extend from proximal side 143 to distal side 144, as shown in FIG. 5A. Such openings 146 may be used to couple a proximal element line, which is discussed further below, to a proximal element 140 for raising and lowering proximal element 140. Each proximal element 140 may also include one or more frictional elements 145 extending from distal side 144. For example, each proximal element 140 may include one or more rows of frictional elements 145 where frictional elements 145 in each row may be aligned in a direction transverse to longitudinal axis A2. Frictional elements 145 in such rows may also be aligned with frictional elements 145 in other rows in a lengthwise direction thereby forming columns of frictional elements 145. For example, in the embodiment depicted in FIGS. 5A and 5B, each proximal element 145 may include four rows of two frictional elements 145. In other words, two columns of four frictional elements 145. In other embodiments, proximal elements 140 may include one to six rows of two to six frictional elements 145 per row, for example. However, in other embodiments, frictional elements 145 may be arranged in an offset relationship in a lengthwise and/or transverse direction such that at least some frictional elements 145 are not aligned with another frictional element 145 in such directions.

Frictional elements 145 may comprise frictional protrusions or tines having tapering pointed tips extending from distal side 144 of proximal elements 140. Frictional elements 145 may also be angled toward fixed end 141a of proximal element 140 which may help prevent frictional elements 145 from inadvertently snaring tissue during repositioning of fixation device 112. In one example, frictional elements 145 may be integral with or connected to a distal surface 144 of a proximal element 140 and protrude therefrom. In another example, as shown in FIG. 5A, frictional elements 145 may be formed from side edges 142, such as by cutting and bending the base material forming proximal elements 140, for example. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 145 be atraumatic and generally frictional rather than penetrative so as to not injure or otherwise affect the tissue in a clinically significant way.

Base section 150 may be connected to a center portion of fixation device 112 such that proximal elements 140 extend outwardly therefrom. For example, base section 150 may be coupled to coupling member 160. In the embodiment depicted, base section 150 may include a first member 152, a second member 154, and a third member 156. First and third members 152, 156 may be connected to second member 154 to form a generally U-shaped or box-shaped structure which may allow a lock (discussed below) to be positioned between first and third members 152, 156. However, other shapes may be formed, such as a V-shape, a crescent shape, or semicircular, for example. In some embodiments, first and third members 152, 156 may be connected to second member 154 via base bend features 157, for example. Also, second member 154 may include an opening 158 extending therethrough for receipt of stud 131 and/or actuator rod 170, as shown in FIG. 5A.

Arm bend features 153 may couple a respective proximal element 140 and base section 150. For example, an arm bend feature 153 can couple a proximal element 140 to first member 152 of base section 150, and another arm bend feature 153 can coupled the other proximal element 140 to third member 156 of base section 150. As shown, arm bend features 153 may form a living hinge about which proximal elements 140 may bend relative to base section 150. In this regard, arm bend features 153 may be integral with proximal elements 140 and base section 150 and may bias proximal elements 140 to a relaxed position. As illustrated in FIG. 5B, proximal elements 140 may form a relaxed angle 149 formed between proximal sides 143 of each proximal element 140. Such relaxed angle 149 is formed when proximal elements 140 are in the relaxed position and may form an angle of about 85 degrees to 200 degrees (±5 degrees). For example, proximal elements 140 may form a relaxed angle of 180 degrees in the relaxed position. In another example, proximal elements 140 may form a relaxed angle of 185 degrees in the relaxed position. Although the embodiment depicted illustrates bend features 153 as living hinges, in other embodiments bend features 153 may comprise a biased hinge that modularly connects proximal elements 140 to base section 150. For example, proximal elements 140 may be separately formed from base section 150 and modularly connected to base section 150 via arm bend features 153 which may each comprise a spring biased hinge biasing a respective proximal element 140 to the relaxed position, for example.

Arm bend features 153 may also each include an elongate opening extending 151 along the longitudinal axis A2 which may furcate each arm bend feature 153, as illustrated in FIG. 5A. Such an elongated opening 151 may have a uniform width extending along axis A2. However, in some embodiments, such as the embodiment depicted, elongate opening 151 may form a bowling-pin shape such that a width of opening 151 is narrower at one end (e.g., the end closest to free end 141b) than the other end (e.g., the end furthers from free end 141b) and is wider somewhere in between. Elongate opening 151 may also not be relegated to just arm bend feature 153 but may also extend from arm bend feature 153 to proximal element 140 and/or base body 150. The elongate opening 151 and corresponding furcation of arm bend features 153 may be configured (e.g., in size, shape, spacing, position, etc.) so as to provide the desired resiliency, fatigue resistance, and/or flexibility at the coinciding arm bend features 153.

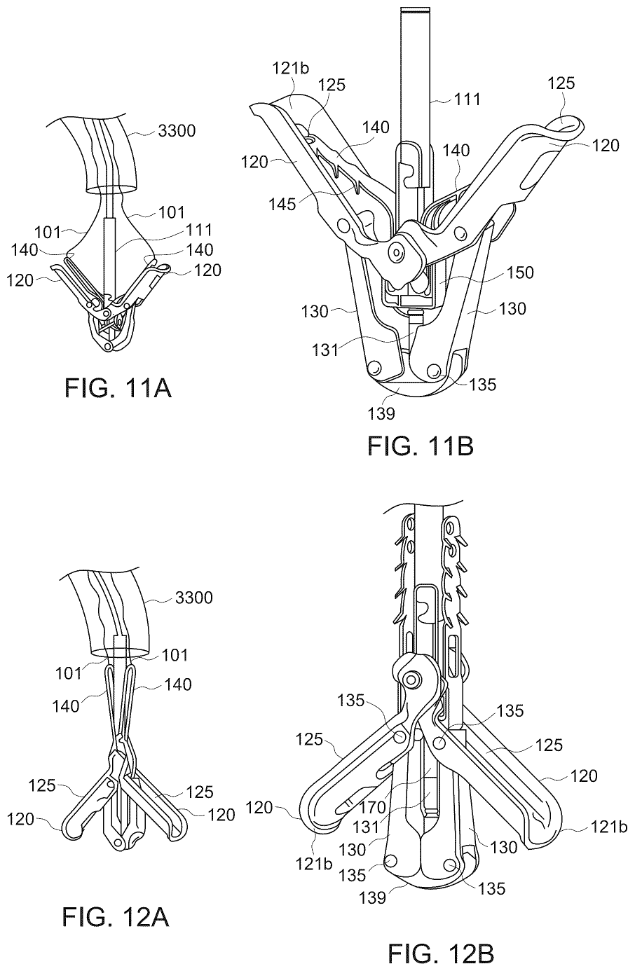

Base bend features 157 and arm bend features 153 may be configured to give gripping device 114 a bent configuration when gripping device is in a relaxed state (i.e., when proximal elements are in the relaxed position), such that when gripping device 114 is forced into a stressed state (e.g., by bending proximal elements at one or more of the base and/or arm bend features 157 and 153), gripping device 114 is resiliently biased toward the relaxed state. In the exemplary embodiment depicted, gripping device 114 may be formed from a metallic sheet of a spring-like material, such as a shape-memory metal (e.g., Nitinol) which may provide the bias of proximal elements 140 toward the relaxed position. Alternatively, elements 140 gripping device 114 could be molded from a biocompatible polymer. Each proximal element 112 may, in one example, be configured to be at least partially recessed within the concavity of the distal element 120 when no tissue is present. When fixation device 112 is in the open position, each proximal element 140 may be separated from the engagement surface 125 near free end 121b of arm 121 and may slope toward engagement surface 125 near free end 121b with the free end 141b of proximal element 140 contacting engagement surface 125, as illustrated in FIGS. 4A and 11B. This arrangement may be facilitated by the dimensions of base section 150. For example, increasing or decreasing the respective lengths of first, second, and third members 152, 154, 156 of base section 150 may increase or decrease the separation distance between a proximal element 140 and corresponding distal element 120 which may help accommodate a valve leaflet or other tissues of varying thicknesses. Further examples of gripping devices that may be utilized in fixation device 112 are described in more detail in U.S. Pat. No. 11,096,691, the disclosure of which is incorporated by reference herein in its entirety.

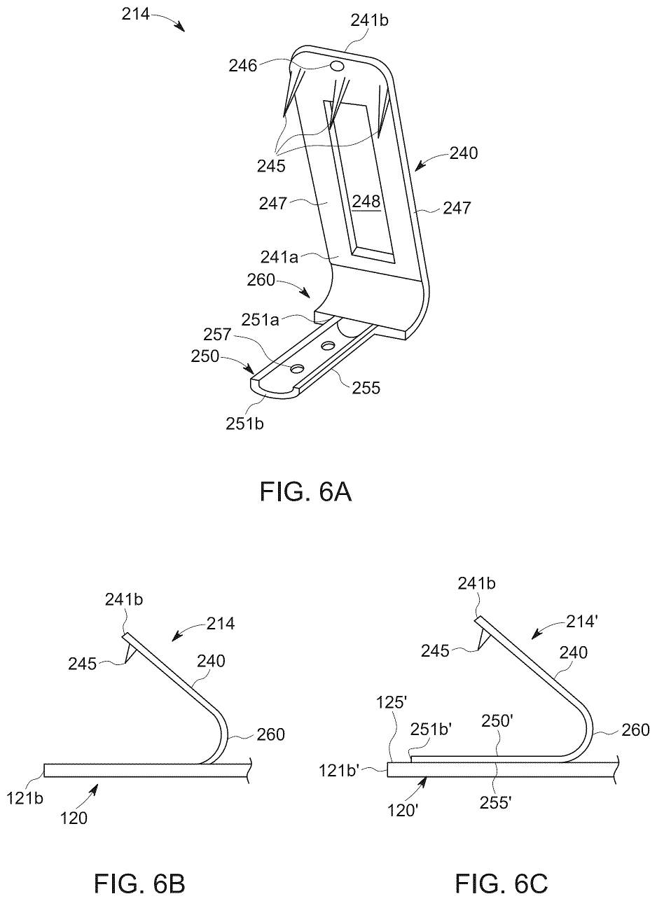

In other embodiments proximal elements may be connected to or otherwise extend from distal elements rather than from a center of fixation device, like that of fixation device 112. For example, FIGS. 6A and 6B depict a gripping device 214 according to another embodiment of the present disclosure that may generally include a first arm 240, a second arm 250, and an arm bend feature 260 partitioning first arm 240 from second arm 250. Gripping device 214 may be made from a memory-metal material, such as Nitinol, for example.

First arm 240 may constitute a proximal element of fixation device 112, like that of and as an alternative to proximal element 140 and may include one or more frictional elements 245 which may be similar to frictional elements 145 discussed above. Thus, a plurality of frictional elements 245 may extend from a distal side of first arm 240 such as in one or more rows and/or columns. In the embodiment depicted, a single row of three frictional elements 245 may be provided near a free end 241b of first arm 240. But, as mentioned above, first arm 240 may have any number of frictional elements 245, such as two, four, or six, for example. First arm 240 may also include a pair of elongate members 247 offset from each other to form a space 248 therebetween. Such space 248 may be configured to receive second arm 250, for example. Additionally, first arm 240 may include one or more openings 246, such as near free end 241b, as shown in FIG. 6A. Such opening 246 may be configured to receive a proximal element line for raising and lowering first arm 240.

Second arm 250 may be in the form of a beam or other elongate structure. Second arm 250 (also referred to herein as base section 250) may be configured to couple to a distal element 120. For example, in the embodiment depicted in FIGS. 6A and 6B, second arm 250 may be curved in a plane transverse to its longitudinal axis. For example, second arm 250 may be semi-cylindrical such that it may have a semi-circular profile. Thus, second arm 250 may have a convex surface 255 configured to conform to the cupped curvature of engagement surface 125 of a corresponding distal element 120. FIG. 6B illustrates second arm 250 coupled to proximal engagement surface 125 of distal element 120 such that it is generally recessed within distal element 120 and free ends 241b, 251b of first and second arms 240, 250 point in the general direction toward free end 121b of distal element 120. Thus, in some embodiments, second arm 250 may have a width configured to be positioned within the concavity of distal element 120 and secure to proximal engagement surface 125. In other embodiments, a second arm 250′ of an alternative gripping device 214′ may not be concave and may instead have a planar surface corresponding to a planar engagement surface 125′ of an alternative distal element 120′ and secured thereto, as illustrated in FIG. 6C. In further embodiments, distal element 120 may include a recess or pocket for receipt and securement of second arm 250, such as in a press-fit manner, for example. Second arm 250 may be secured to distal element 120 in any number of ways, such as via one or more sutures, welding, press-fit, fastener (e.g., rivet or screw) or the like. For example, a rivet, screw, or suture may pass through one or more openings 257 in second arm 250 and into distal element 120. A tissue fixation device, such tissue fixation device 112, may include a pair of gripping devices 214 with one coupled to each distal element 120 as mentioned above.

Arm bend feature 260 may be coupled to a fixed end 241 of first arm 240 and a fixed end 251a of second arm 250 such that first and second arms 240, 250 extend in the same general direction and may form a V-shape when first arm 240 is in an exemplary open or raised position, as illustrated in FIGS. 6B and 6C. As shown, arm bend feature 260 may form a living hinge about which first arm 240 may bend relative to second arm 250. In this regard, arm bend feature 260 may be integral with first arm 240 and second arm 250 so as to form a monolithic structure and may bias first arm 240 to a relaxed position. Such relaxed position may include second arm 250 extending through space 248 between elongate members 247 of first arm 240 to form an X-shape. However, it should be noted that such position can generally only be achieved when gripping device 214 is not coupled to distal element 120 as the presence of distal element 120 would prevent second arm 250 from passing into space 248. It should also be appreciated that in some embodiments of gripping device 214, arm bend feature 260 may be a spring loaded or otherwise biased hinge coupling separately formed first and second arms 240, 250.

Fixation device 114 may also have a covering 117, as shown in FIG. 4B. As depicted, covering 117 may encapsulate distal elements 120 and actuation mechanism 113. Thus, engagement surfaces 125 may be covered by covering 117 which may help minimize trauma on tissues and enhance primary fixation via additional friction to assist in grasping. Additionally, covering 117 on engagement surfaces 125 may facilitate tissue ingrowth to provide for secondary fixation to ensure long-term security. Covering 117 may be loosely fitted and/or may be flexible such that device 112 can freely move to various positions all the while covering 117 conforms to the contours of the device 112 and remains securely attached thereto. It may be appreciated that the covering 117 may cover specific parts of fixation device 112 while leaving other parts exposed. For example, proximal elements 140 may be exposed, while distal elements 120 and actuation mechanism 113 may be covered. However, in some embodiments, proximal elements 140 may be covered with covering 117 to enhance grip and tissue ingrowth following implantation. Preferably, when a covering 117 is used in combination with frictional elements 145 or other frictional features, such as those extending from proximal elements 140, such features may protrude through such covering 117 so as to contact any tissue engaged by proximal elements 140.

Covering 117 may be comprised of any biocompatible material, such as polyethylene terepthalate, polyester, cotton, polyurethane, expanded polytetrafluoroethylene (ePTFE), silicon, or various polymers or fibers and have any suitable form, such as a fabric (woven or unwoven), mesh, textured weave, felt, looped or porous structure. Generally, covering 117 has a low profile so as not to interfere with delivery through an introducer sheath or with grasping and coapting of leaflets or tissue. Covering 117 may alternatively be comprised of a polymer or other suitable materials dipped, sprayed, coated, or otherwise adhered to the surfaces of the fixation device 112. Optionally, a polymer coating may include pores or contours to assist in grasping the tissue and/or to promote tissue ingrowth. Any of the coverings 117 may optionally include drugs, antibiotics, anti-thrombosis agents, or anti-platelet agents such as heparin, COUMADIN® (Warfarin Sodium), to name a few. These agents may, for example, be impregnated in or coated on the coverings 117. These agents may then be delivered to the grasped tissues surrounding tissues and/or bloodstream for therapeutic effects.

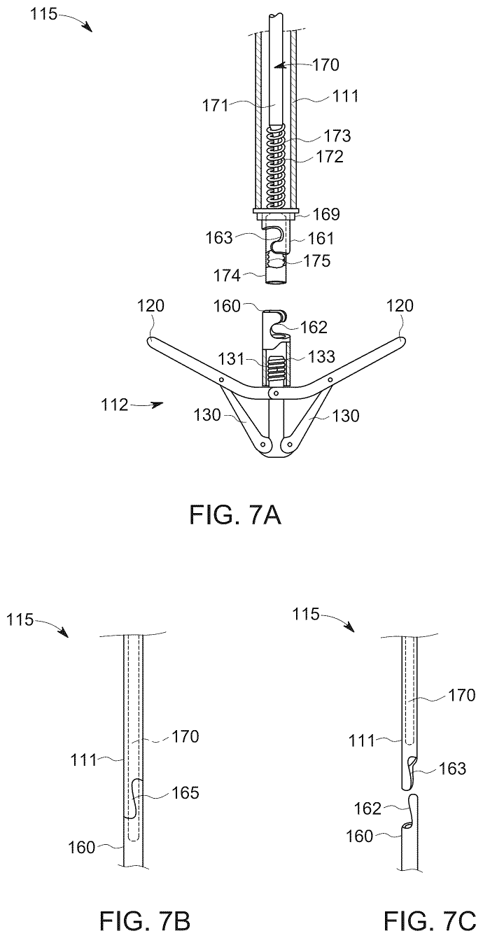

FIGS. 7A-7C depict an exemplary coupling system 115 between fixation device 112 and delivery system shaft 111. As mentioned above, once the leaflets of a target valve are coapted in the desired arrangement, fixation device 112 may then be detached from delivery system 1000 and left behind as an implant to hold the leaflets together in the coapted position. Such detachment may occur between coupling member 160 of fixation device 112 and a distal end of delivery shaft 111. Thus, coupling member 160 may be configured to be releasably coupled to shaft 111. Coupling member 160 may be disposed at a center of fixation device 112 and may extend proximally along it's the longitudinal axis of fixation device 112. In the coupling system 115 depicted, shaft 111 may form a tubular upper shaft with a first mating surface 163 formed at a distal end thereof, and coupling member 160 may form a detachable lower tubular shaft with a second mating surface 162 formed at a proximal end thereof. Mating surfaces 162, 163 may be correspondingly shaped so that they interlock and form a joining line 165 when merged together, as shown in FIG. 7B. In this regard, mating surfaces 162, 163 may have any shape or curvature which allows or facilitates interlocking and later detachment. For example, in the depicted embodiment, mating surfaces 162, 163 define a joining line 165 with an S-shaped curvature.

Coupling system 115 may also include actuator rod 170 and stud 131 (or alternatively base 139) such that fixation device 112 may also be releasably coupled to delivery system 1000 via connection between actuator rod 170 and stud 131. When shaft 111 is coupled to coupling member 160, they may collectively form an axial channel. Actuator rod 170 may pass through this channel to bridge the joining line 165, as shown in FIG. 7B. Actuator rod 170 may comprise a proximal extremity 171, a distal extremity 172, and a joiner 174. Distal extremity 172 may be smaller in diameter than proximal extremity 171 and may be optionally surrounded by a coil 173 which may serve to bias joiner 174 in a proximal direction. However, in some embodiments, actuator rod 170 may not have coil 173 or proximal and distal extremities 171, 172 of differing diameters. Joiner 174 may be removably coupled with stud 131 of fixation device 112 via any one of various possible release mechanisms. For example, in the embodiment depicted, joiner 174 may be threadedly connected to stud 131 of fixation device 112. In this regard, joiner 174 may have internal threads 175 which mate with external threads 133 on stud 131. Alternatively, joiner 174 may have external threads which mate with internal threads of stud 131. As described previously, stud 131 may be connected with distal elements 120 so that advancement and retraction of stud 131, by means of actuator rod 170, manipulates distal elements 120. It is also contemplated that joiner 174 may be directly threadedly engaged with base 139 where no stud 131 is provided. Once detachment of fixation device 112 is desired, actuator rod 170 may be rotated until threads 175 of joiner 174 disengage threads 133 of stud 131. Actuator rod 170 may then be retracted to a position above mating surfaces 162, 163 which in turn allows coupling member 160 to separate from shaft 111 along joining line 165, as illustrated in FIG. 7C.

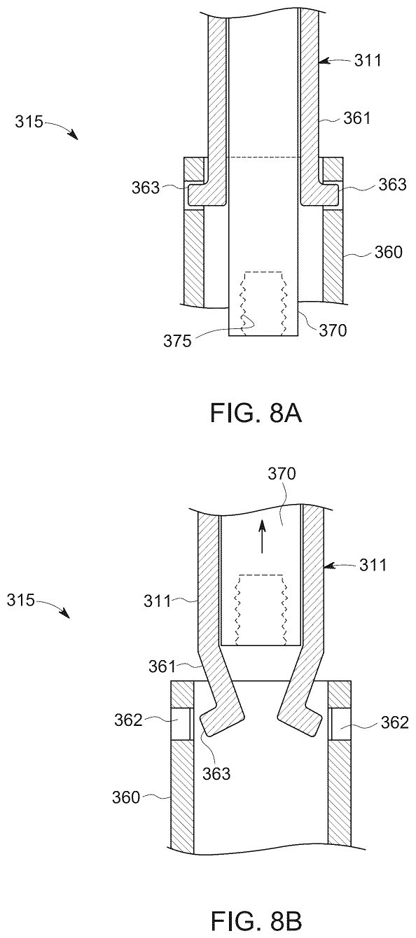

FIGS. 8A and 8B illustrate an alternative example of a coupling system. In this exemplary coupling system 315, shaft 311 of the delivery system (e.g., delivery system 1000) may be releasably coupled with coupling member 360 via a detent mechanism, for example. In this regard, shaft 311 may form an upper tubular shaft with detent mechanism features and coupling member 360 may form a lower tubular shaft with detent mechanism features configured to releasably connect with the detent mechanism features of shaft 311. In the embodiment depicted, the detent mechanism may include one or more spring arms 361 integrally formed on shaft 311 and one or more receptacles 362 sized to receive spring arms 361 within coupling member 360. However, shaft 311 may include receptacles 362, while coupling member 360 may include spring arms 361, for example. As shown, spring arms 361 may have a flange-like engagement element 363 at a distal end thereof and are preferably biased inwardly, i.e., toward an interior shaft 311, as shown in FIG. 8B. Receptacles or apertures 362 may be configured to receive and mate with respective engagement elements 363 of spring arms 361, as shown in FIG. 8A. Receptacles 362 may extend all the way through the wall of coupling member 360 and may be sized to snuggly fit both engagement elements 362. A snuggly fitting rod (such as actuator rod 370) may extend through shaft 311 and coupling member 360 and may outwardly deflecting the inwardly biased spring arm(s) 361 such that the engagement elements 363 are pushed into respective engagement with a corresponding receptacle 362 thereby coupling the shaft 311 to coupling member 360, as shown in the example of FIG. 8A. When desirable to detach fixation device 112 from delivery system 1000, actuator rod 370 may be retracted to a position above spring arm(s) 361 and engagement features 363 thereof. This allows the inwardly biased spring arms 361 and corresponding engagement elements 363 to disengage from receptacles 362 thereby detaching shaft 311 and coupling member 360. As mentioned above, actuator rod 370 may be threadedly engaged to stud 131. Thus, actuator rod 370 may first be rotated to unthread its threads 375 from stud 131 and then retracted to release coupling member 360 according to an example of the disclosure.

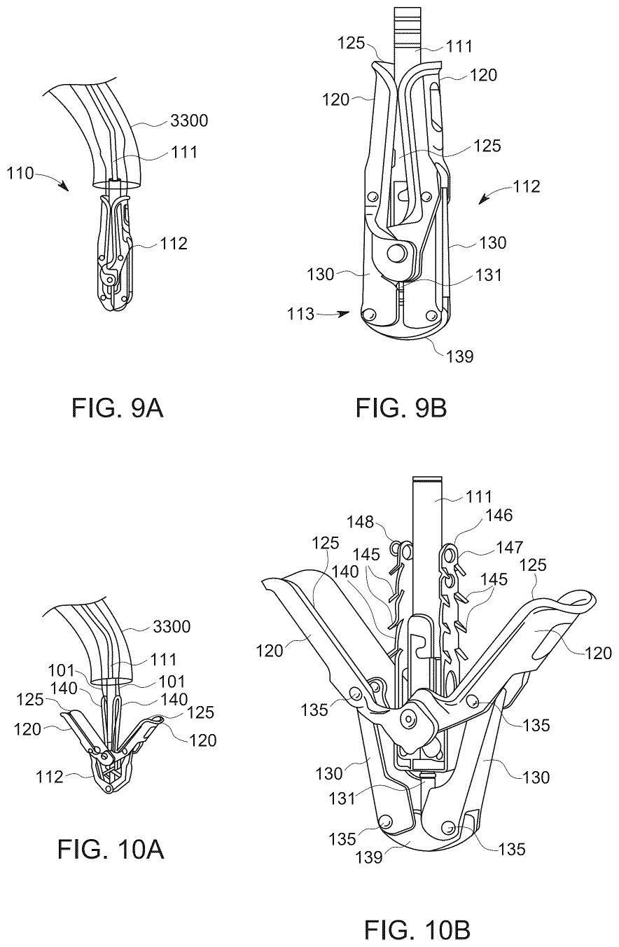

As mentioned above, fixation device 112 may, in one example, be actuated through multiple positions within a mammalian body during a transcatheter procedure such as by extending and retracting actuator rod 170 when coupled to stud 131 and/or base 139. FIGS. 9A-9B, 10A-10B, 11A-11B, 12A-12B, and FIGS. 13A-13B illustrate several of these possible positions and in a sequence that may be utilized during a transcatheter procedure.

FIGS. 9A and 9B depict fixation device 112 in an example of a closed position or delivery position. Fixation device 112 may assume the closed position when being delivered through a guide catheter or sheath 3300 of steerable guide system 5, as shown in FIG. 9A. In the closed position, the opposed pair of distal elements 120 may be positioned so that engagement surfaces 125 thereof face each other. The cupped or concave shape of each arm 121 in this example allows arms 121 to surround shaft 111 and optionally contact each other on opposite sides of shaft 111. This provides a low profile for fixation device 112 so that it is readily passable through catheter 3300 and through any anatomical structures, such as those within the cardiovascular system.

FIGS. 10A-10B depict fixation device 112 in an example of an open position. Fixation device 112 may assume the open position for capturing and grasping leaflets of a heart valve. In an open position, distal elements 120 may be rotated so that engagement surfaces 125 thereof face a first direction such that engagement surfaces 125 are disposed at an acute angle relative to shaft 111. For example, the acute angle formed between each engagement surface 125 and shaft may be 45 degrees to 90 degrees. Stated differently, in the open position, engagement surfaces 125 of distal elements 120 may be oriented 90 degrees to 180 degrees relative to each other. However, it is generally preferable for arms to be positioned 120 degrees relative to each other (and 60 degrees relative to shaft 111) for capturing leaflets. Movement of fixation device 112 from the closed position to the open position may be achieved by advancing stud 131 distally relative to coupling member 160 by distally advancing actuator rod 170. Conversely, fixation device 112 may be moved from the open position to the closed position by retracting actuator rod 170 and retracting stud 131 proximally, according to one example of the disclosure.

As shown in FIG. 10B, proximal elements 140 (or proximal elements 240) may be in a raised or insertion position when fixation device 112 is in the open position to facilitate insertion of leaflets between distal and proximal elements 120, 140 for their capture. Proximal elements 140 are, in one example, biased toward distal elements 120. In this regard, proximal elements 140 may be moved inwardly toward shaft 111 and held against shaft 111 with the aid of proximal element lines 101 which can be in the form of sutures, wires, nitinol wire, rods, cables, polymeric lines, or other suitable structures, as shown in FIG. 10A. Thus, FIGS. 10A and 10B depict fixation device 112 in an insertion configuration in which proximal elements 140 are in a raised position and distal elements 120 are in an open position.

Once fixation device 112 has been positioned in a desired location against the valve leaflets, the leaflets may then be captured between proximal elements 140 and distal elements 120. FIGS. 11A and 11B illustrate fixation device 112 in an example of such a position. Here, proximal elements 140 are lowered toward engagement surfaces 125 so that proximal elements 140 are in a lowered or capture position, and the leaflets are held between distal and proximal elements 120, 140. Proximal elements 140 are, in one example, lowered into the lowered position while distal elements 120 remain in the open position. Thus, fixation device 112, as shown in FIGS. 11A and 11B is in an example of a capture configuration which may be similar to the insertion configuration of FIGS. 10A and 10B, but with the difference being that proximal elements 140 are now lowered toward distal elements 120 by releasing tension on proximal element lines 101 to compress the leaflet tissue therebetween. At any time, the proximal elements 140 may be raised and the distal elements 120 adjusted or inverted to reposition fixation device 112 if regurgitation is not sufficiently reduced according to one example of the disclosure.

FIGS. 12A-12B depict an example of an inverted position of fixation device 112. Fixation device 112 may assume the inverted position to aid in repositioning or removal of fixation device 112. In one example of the inverted position, distal elements 120 may be further rotated from the open position, which may be achieved by advancing stud 131 further relative to the open position, so that the engagement surfaces 125 of distal elements 120 face outwardly, and free ends 121b point distally. Additionally, in some examples, engagement surfaces 125 of each arm 121 may form an obtuse angle relative to shaft 111. For example, the obtuse angle formed between each engagement surface 125 and shaft 111 may be 135 degrees to 180 degrees. Stated differently, in the inverted position, engagement surfaces 125 of distal elements 120 may be oriented 270 degrees to 360 degrees relative to each other.

Also, as shown in FIG. 12B, in one example proximal elements 140 are in their raised position against shaft 111 while distal elements 120 are in the inverted position by exerting tension on the proximal element lines 101. Thus, a relatively large space may be created between proximal and distal elements 140, 18 for repositioning. In addition, the inverted position allows withdrawal of the fixation device 112 through the valve while minimizing trauma to the leaflets. Engagement surfaces 125 provide an atraumatic surface for deflecting tissue as the fixation device is retracted proximally. It should be further noted that tines 145 of proximal elements 140 may, in some examples, be angled slightly in the distal direction (away from the free ends of the proximal elements 140), reducing the risk that tines 145 will catch on or lacerate tissue as fixation device 112 is withdrawn and while proximal elements 140 are in the raised position.

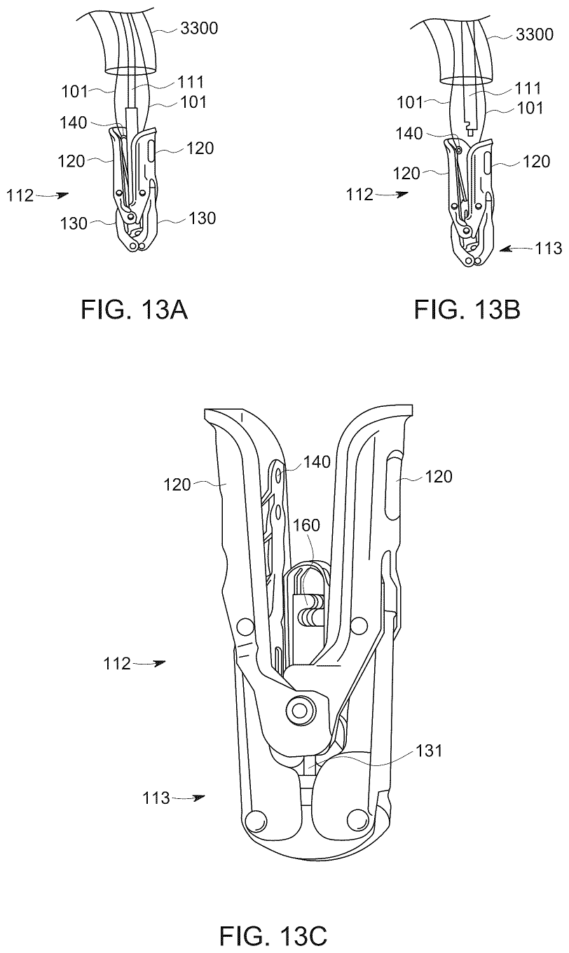

After the leaflets have been captured between distal and proximal elements 120, 140, distal elements 120 may be returned to or toward the closed position where they may be locked in place. An example of such locking is described further below. FIG. 13A illustrates fixation device 112 in the closed position wherein the leaflets (not shown) are captured and coapted. In one example, this is achieved by retraction of the stud 131 proximally relative to coupling member 160 so that the legs 130 of the actuation mechanism 113 apply an upwards force to distal elements 120 which in turn rotate distal elements 120 so that engagement surfaces 125 again face one another, similar to that of FIGS. 9A and 9B, and so that distal elements 120 rotate proximal elements 140 in a direction toward shaft 111. However, because the leaflets are captured between distal and proximal elements 120, 140, it may be desirable to keep distal elements 120 at about 20 degrees to 60 degrees relative to each other so as to limit the amount of tension and stress on the native tissue. Thus, while fixation device 112 may be returned to the closed position, such closed position may not be as closed as in the initial delivery position.

As shown in FIG. 13B, fixation device 112 may then be released from shaft 111 of delivery system 1000 while in the closed position. As mentioned, fixation device 112 may be releasably coupled to delivery system 1000 via a coupling system (e.g., coupling system 115 or 315). When the coupling structures of such coupling system are released, proximal element lines 101 may remain attached to proximal elements 140 following detachment to function as a tether to keep the fixation device 112 connected with the delivery catheter 1020 (see FIG. 42B) for reconnection and repositioning. However, in other embodiments, proximal elements lines 101 may be released prior to release of fixation device 112 or concurrently with the release of fixation device 112, as described in more detail below.

FIG. 13C illustrates a released fixation device 112 in an example of a closed position. As shown, coupling member 160 remains separated from shaft 111 of delivery system 1000, and proximal elements 140 are deployed so that tissue (not shown) may reside between proximal elements 140 and distal elements 120.

As mentioned above, proximal element lines or actuators 101 may be releasably coupled to proximal elements 140. In some examples, proximal element lines 101 may pass through an opening in proximal elements 140, such as openings 146 and 246 in the case of proximal element 240. In other examples, eyelets, which may be formed from one or more lengths of suture, may be coupled to proximal elements 140 and proximal element lines 101 may pass through such eyelets. Thus, proximal element lines 101 may be released from proximal elements 140 prior to, concurrent with, or after release of fixation device 112 from delivery system 1000 according to various examples.

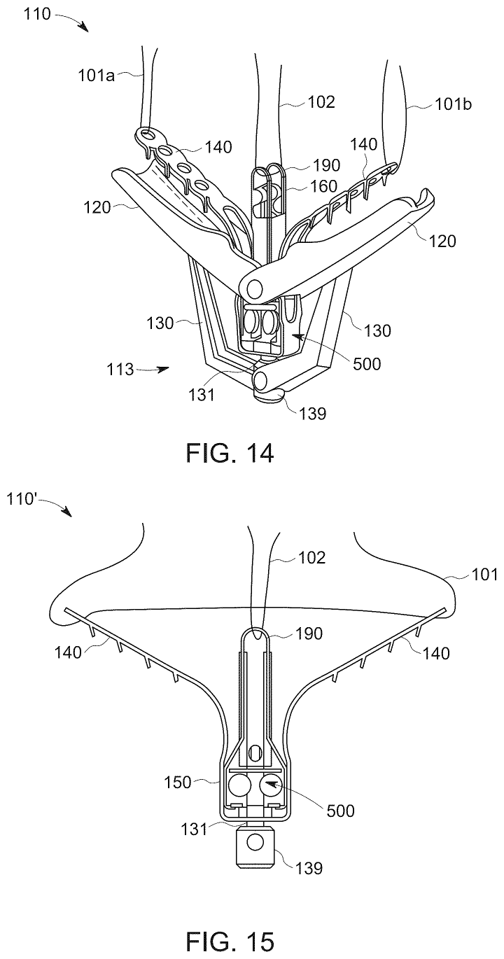

In an exemplary embodiment of interventional tool 110, as shown in FIG. 14, a plurality of proximal element lines 101a, 101b may be coupled to proximal elements 140 of fixation device 112. Each of proximal element lines 101a and 101b may be elongated flexible threads, wire, cable, sutures, or lines extending through shaft 111, looped through proximal elements 140, and extending back through shaft 111 to a delivery device handle 1010 (see FIG. 42A) of delivery system 1000. When detachment is desired, one end of each proximal element line 101a, 101b may be released from delivery system 1000, and the other end pulled to draw the free end distally through shaft 111 and through proximal element 140 thereby releasing it. Also, in this arrangement, proximal element lines 101a and 101b may be independently or concurrently manipulated so as to independently or concurrently raise and lower proximal elements 140, respectively.

In another example, interventional tool 110′ may be configured, as shown in FIG. 15 with respect to certain components thereof, such that proximal elements 140 may alternatively be supported by a single proximal element line 101 which may extend through both of the proximal elements 140. In this arrangement both proximal elements 140 may be raised and lowered concurrently by action of a single proximal element line 101. Whether proximal elements 140 are manipulated individually by separate proximal element lines 101 or jointly by a single proximal element line 101, the proximal element lines 101 may extend directly through openings (e.g., openings 146, 246) of the proximal elements 140 and/or through a layer or portion of a covering 117 on proximal elements 140, or through a suture loop/eyelet above or below a covering 117, for example.

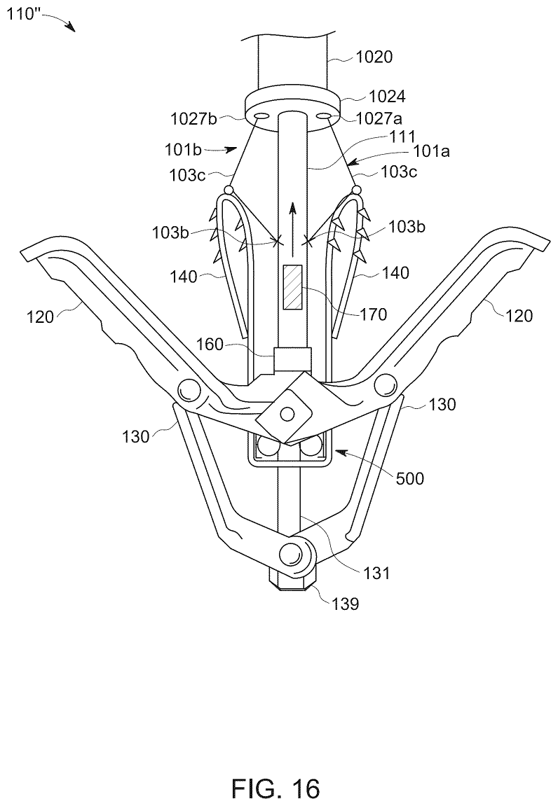

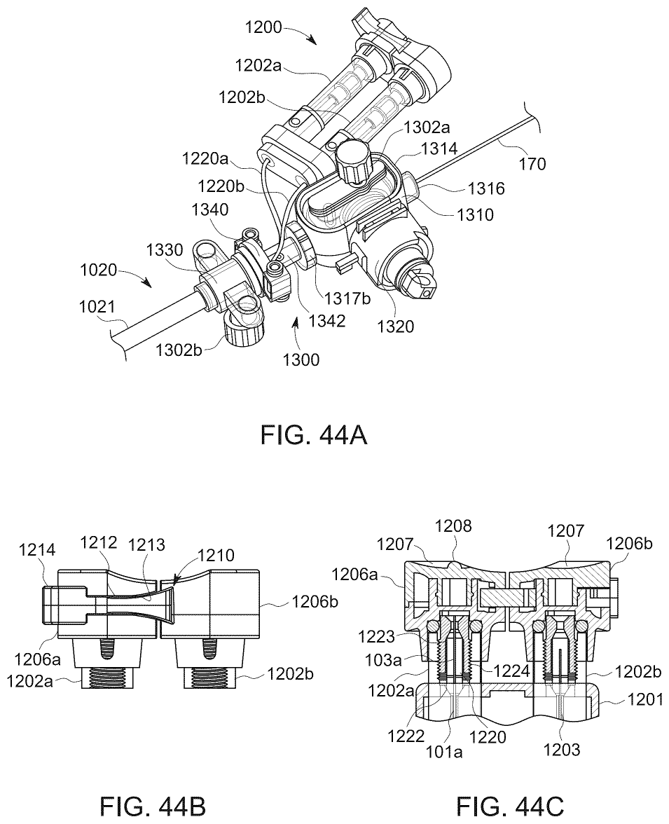

In a further example, interventional tool 110″ may be configured, as shown in FIG. 16, such that each proximal element line 101a, 101b may be releasably engaged with structures that are activated by removal of the actuator rod 170 that passes through coupling member 160 and shaft 111 such that release of proximal element lines 101a, 101b occurs concurrently with the release of fixation device 112 from delivery system 1000. Thus, in one example, which is depicted in FIG. 16, each proximal element line 101a, 101b may have a first end portion 103a (e.g., proximal end portion), which may be coupled to an actuator of a delivery system handle 1010 (see e.g., FIG. 44C), a second end portion 103b (e.g., distal end portion) which may be releasably engages to shaft 111 via actuator rod 170, and an intermediate portion 103c which may be coupled to a proximal element 140. As described above and as illustrated in FIG. 7A, stud 131 may be releasably attached to actuator rod 170 which passes through coupling member 160 and shaft 111 of delivery system 1000. In this way, actuator rod 170 is connectable with fixation device 112 and acts to manipulate fixation device 112 so as to move it through its various positions, which are described above. After the leaflets have been coapted, actuator rod 170 may be removed proximally from stud 131 which may thereby also release coupling member 160 from shaft 111, as described with respect to FIGS. 7A-7C and also FIGS. 8A and 8B with respect to coupling system 315. This action of actuator rod 170 may be utilized to release distal end portion 103b of each of proximal element lines 101a, 101b.

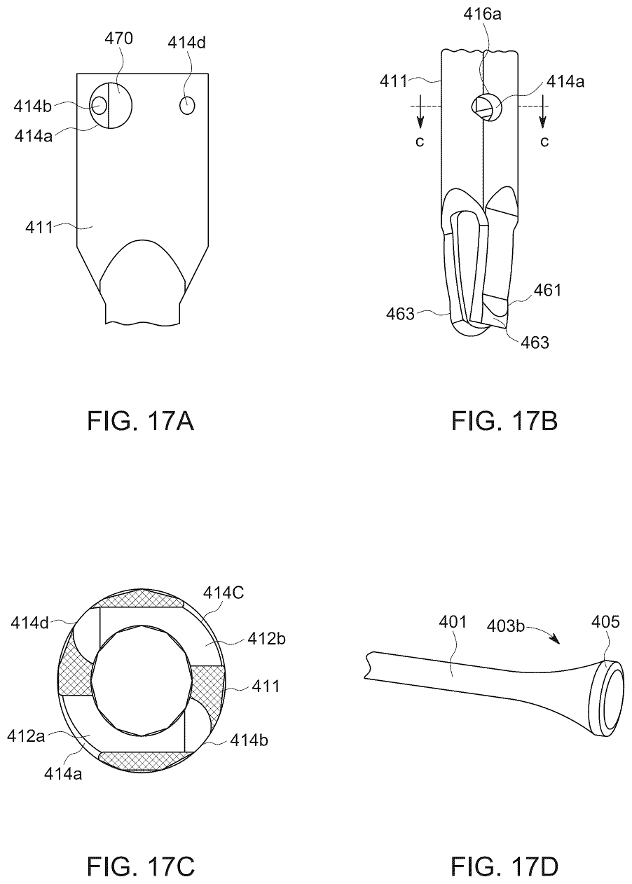

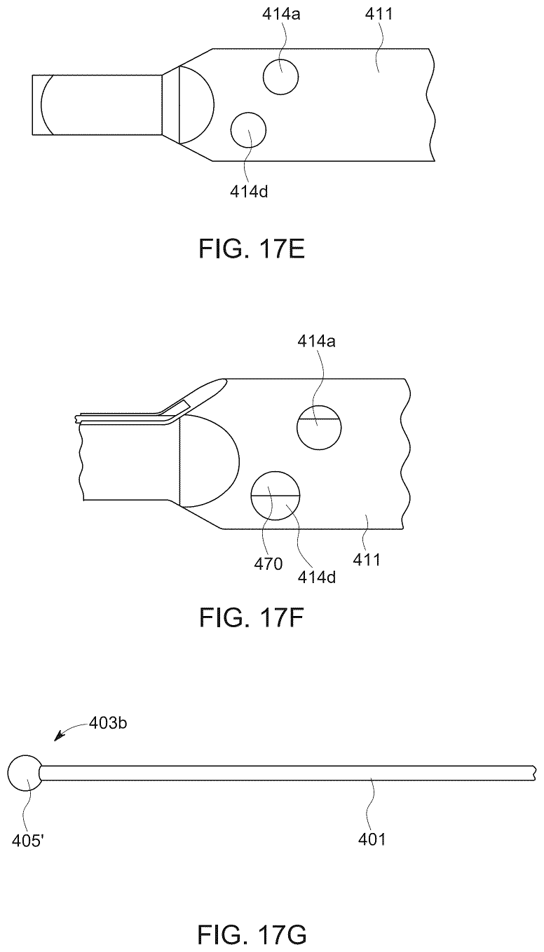

Exemplary features which may be implemented in interventional tool 110″ to facilitate release of proximal element lines 101a, 101b in this manner are shown in FIGS. 17A-17G. As depicted, an actuator rod 470 may be used as an anchor to restrict proximal movement of one or more proximal element lines 401. Proximal element line 401 has a distal end portion 403b which may include a catch element 405, for example a trumpet 405 having a cone shape (see FIG. 17D) or other shapes, such as a ball 405′ having a spherical shape (see FIG. 17G), which can be sized to be received within shaft 411. As shown in the example of FIGS. 17B, shaft 411 may have spring arms 461 like that of the coupling system 315 of FIGS. 8A and 8B for releasing device 112 from shaft 411. However, shaft 411 may also have mating surfaces of FIGS. 7A and 7C. In any event, a portion of shaft 411 proximal of spring arms 461 (or mating elements 463), may have two slots 412a and 412b defined therein. Slot 412a can define holes 414a and 414b and slot 412b can define holes 414c and 414d. Holes 414a and 414c can be sized to receive catch element 405 of a pair of proximal element lines 401, respectively, therethrough and into slots 412a and 412b, respectively. Holes 414b and 414d can be sized to prevent catch element 405 of proximal element lines 401, respectively, from extending beyond slots 412a and 412b, respectively. The configuration of slots 412a and 412b and holes 414a-414d can allow for easier manufacture of the features in shaft 411. Slots 412a and 412b can be drilled to ensure that slots 412a and 412b do not pass the entire way through shaft 411. In this example configuration, catch elements 405 of proximal element lines 401 can be maintained within shaft 411 to manage the slack of proximal element lines 401.

In one example, catch element 405 of proximal element line 401 can be inserted into slot 412a through hole 414a beyond a longitudinal axis of shaft 411 and toward hole 414b, and catch element 405 of proximal element line 401 can be inserted into slot 412b through hole 414c beyond the longitudinal axis of shaft 411 and toward hole 414d prior to the insertion and coupling of the actuator rod 470 (which passes through shaft 411) with stud 131 of fixation device 112. With actuator rod 470 extending through shaft 411, actuator rod 470 may directly engage catch elements 405 of lines a plurality of proximal element lines 401 thereby preventing their movement back out along the path through which they were inserted. For example, trumpets 405 can be inhibited from being advanced through holes 414b and 414d, respectively, and can be prevented from being pulled past actuator rod 470 and through holes 414a and 414c, respectively. Accordingly, the second end portions 403b of proximal element lines 401 can be held in place relative to shaft 414. Once the actuator rod 470 is decoupled from stud 131 and subsequently retracted, movement of catch elements 405 at the distal end portions of proximal element lines 401 is no longer restricted and proximal element lines 401 are free to move. Upon proximal retraction, proximal element lines 401 can thread through holes 414a and 414c, respectively, and decouple from the proximal elements 140.

In accordance with one example of the disclosed subject matter, slots 412a and 412b can be drilled at an angle towards the distal end of shaft 411 (see FIGS. 17E and 17F), e.g., with hole 414b formed distal to hole 414a on one side, and hole 414d formed distal to hole 141c on the other side. This example configuration of slots 412a and 412b can provide easier deployment of a plurality of proximal element lines 401 and can reduce friction.

Prior to securing second end portion 403b of each proximal element line 401 with the shaft 411, each proximal element line 401 can be coupled with a respective proximal element 140, such as in the manner described above with respect to FIG. 16. Thus, when proximal element lines 401 are actuated proximally, proximal element lines 401 can move proximal elements 140 relative to distal elements 120, thereby moving proximal elements 140 between their respective raised and lowered positions.

As mentioned above, fixation device 112 optionally includes a lock (e.g., lock 500) for locking device 112 in a particular position, such as in any one of the aforementioned open, closed, and inverted positions or any position therebetween. It may be appreciated that according to various examples, the lock may be configured for both locking and unlocking which correspondingly allows device 112 to be both locked and unlocked. As described in more detail below with respect to various lock examples, such locks may have components disposed between coupling member 160 and base 139 which may be configured to selectively arrest proximal-distal movement of stud 131/base 139 which consequently arrests movement of distal elements 120. Such locks may help provide end user control of the final arm angle of fixation device 112 for tailored and optimal results for each patient. Additionally, such locks in conjunction with distal and proximal elements 120, 140 may bring the leaflets and annulus together in a secure relationship which may result in beneficial dimensional changes of the target valve which can prevent adverse remodeling of the heart, particularly for patients with heart failure.

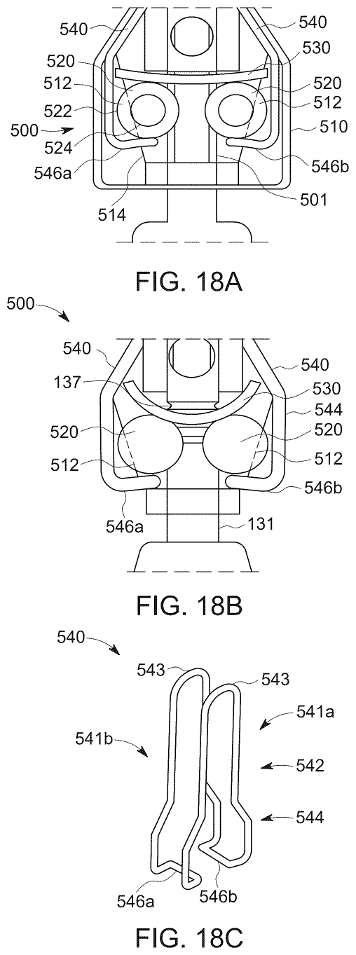

FIGS. 14-16, and 18A-18C illustrate an embodiment of the lock 500. Lock 500 generally includes a housing 510, one or more wedging elements 520, a release harness 540, and a biasing element 530. Housing 510 may be positioned distal to coupling member 160 and may be free-floating, coupled to, or integral with coupling member 160, such as at a distal end thereof. Housing 510 may form a window 514 which may be defined at opposite sides with sloping or tapered surfaces 512 which slope inwardly toward stud 131 in a proximal to distal direction. Wedging elements 520 may be in the form of rolling elements, such as a pair of barbells, disposed on opposite lateral sides of stud 131 and between sloping surfaces 512, as shown in FIGS. 18A and 18B. Each barbell 520 may have a pair of end caps 522 and a shaft or bar 524 therebetween, as illustrated in the barbell cross-section of FIG. 18A. Barbells 520 and stud 131 are preferably comprised of cobalt chromium or stainless steel, however any suitable material may be used. Biasing element 530 may be a spring, such as a leaf spring, for example, and may be positioned at a proximal end of housing 510 between sloping surfaces 512 and proximal to barbells 520 such that spring 530 bears on barbells 520 and biases them in a distal direction. Thus, when barbells 520 are pushed distally by spring 530, they are correspondingly pushed inwardly and wedged against stud 131 by sloping surfaces 512, as illustrated by FIG. 18A, which depicts barbells 520 in a proximal and unlocked position, and FIG. 18B, which depicts barbells 520 in a distal and locked position.

As shown in FIGS. 14, 15, and 18C, release harness 540 may be in the form of a ridged wire or rod that may extend proximally from between base 139 and coupling member 160 toward a proximal end of fixation device 112. The wire that may form harness 540 may have a diameter of 0.007 inches (0.178 mm), for example. Release harness 540 may have a first section or front section 541a and a second section or rear section 541b which, when coupled to fixation device 112, are respectively disposed at a front and rear of stud 131.

Harness 540 may also include a first end portion or proximal portion 542 and a second end portion or distal portion 544. In the embodiment depicted, proximal portion 542 of each section 541a, 541b of harness 540 may include a crest or closed end 543 which may define a proximal terminal end of harness 540. However, in some embodiments, only one of sections 541a and 541b may include a closed end 543. Closed end 543 may be formed by a curved segment of harness 540 which may define a half-loop through which a lock line 102 may be threaded and engaged, as described below. Thus, where each section 541a, 541b includes a closed end 543, lock line 102 may be threaded through each closed end 543.

Distal portion 544 of harness 540 may include a pair of feet or inward extensions 546a, 546b that extend inwardly toward each other and at opposite sides of a longitudinal axis of harness 540. In this regard, harness 540 may be referred to herein as a double-sided harness (“DSH”) or double-foot harness. In the embodiment depicted, feet 546a, 546b may be bent inwardly, such as by bending the wire that may form harness 540. However, in other embodiments, feet 546a, 546b may be separately connected to harness 540 rather than integral therewith. Additionally, as illustrated in FIG. 18C, feet 546a, 546b may extend between and connect first and second sections 541a, 541b. Thus, when harness 540 is coupled to fixation device 112, feet 546a, 546b may be positioned at opposite lateral sides of stud 139 and may extend front to rear between sloping surfaces 512 of housing 510, as shown in FIGS. 18A and 18B. Feet 546a, 546b may be moveable proximally-distally within window 514 and may be positioned distal of barbells 180 such that pulling up on harness 540 moves feet 546a, 546b proximally so as to push the respective barbells 520 against the bias of spring 530 and move them away from stud 131 to their unlocked position.

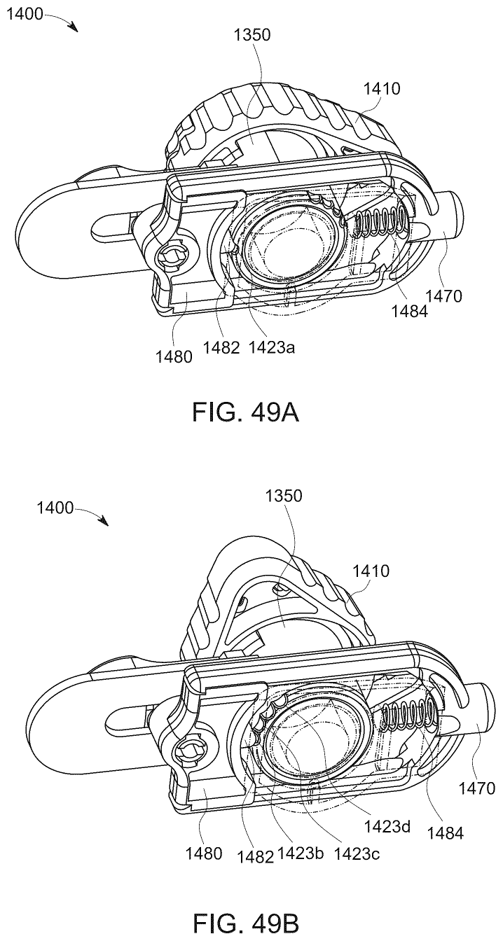

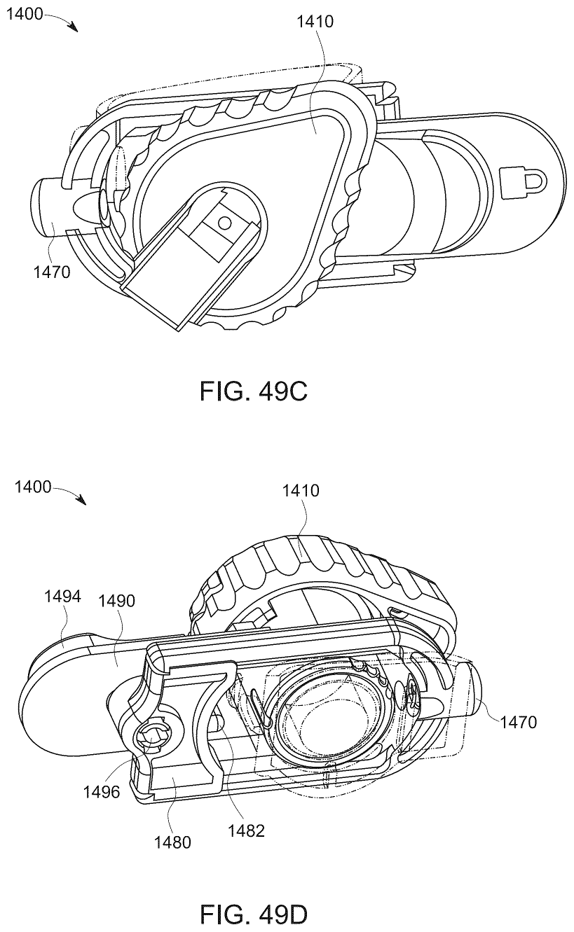

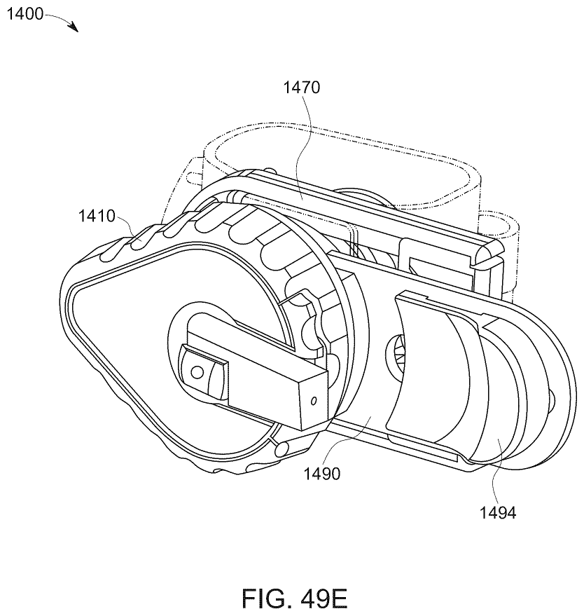

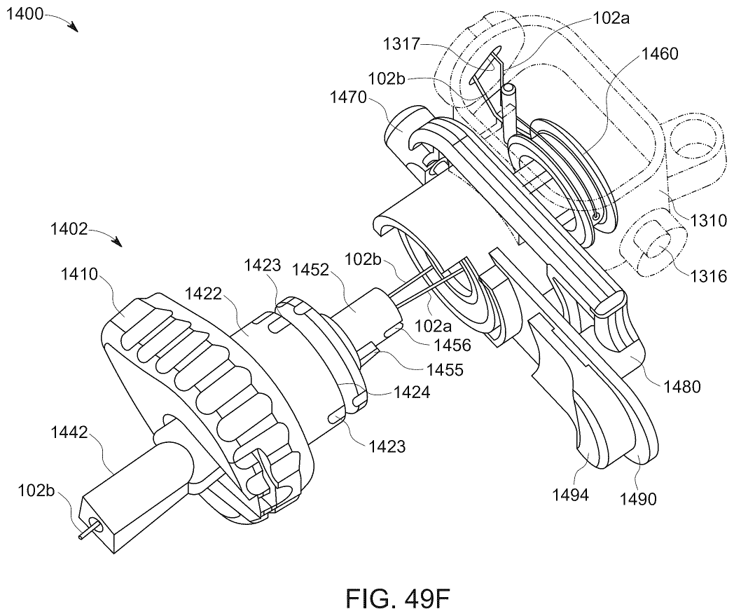

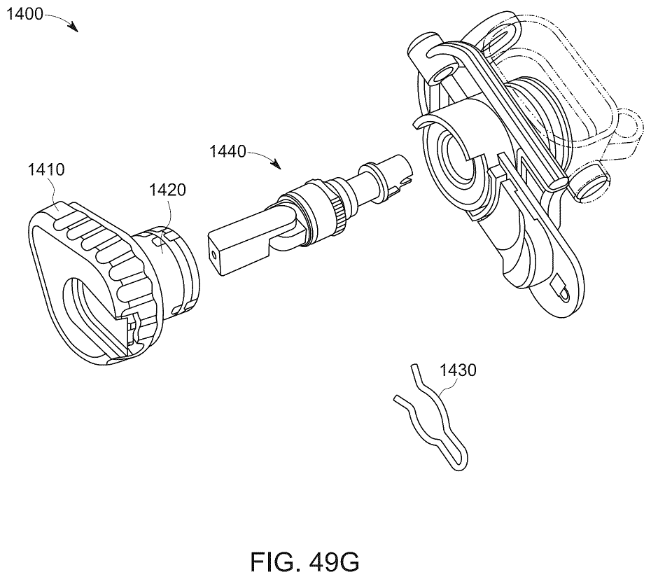

Movement of harness 540 may be performed by one or more lock line 102 which may be coupled to harness 540 such as by threading lock line 102 through closed end 543 of proximal portion 542 of harness 540, as shown in FIGS. 14 and 15. Such lock line 102 may have a first end portion 102a fixedly secured to delivery system handle 1010 of delivery system 1000 and a second end portion 102b releasably secured to delivery system handle 1010 (see e.g., FIG. 49F), as described in more detail below. In this regard, tension can be selectively applied to lock line 102 to unlock and lock lock 500. Also, lock line 102 can be released from release harness 540 prior to, concurrently with, or after release of fixation device 112 from delivery system 1000 which may be achieved by releasing the second end 102b from delivery system handle 1010 and pulling lock line 102 and its second end through shaft 111. Lock line 102 may be comprised of any suitable material, typically wire, nitinol wire, cable, suture, or thread, to name a few. In addition, lock line 102 may include a coating, such as parylene. Parylene is a vapor deposited pinhole free protective film which is conformal and biocompatible. It is inert and protects against moisture, chemicals, and electrical charge.

When an upwards force is applied to harness 540 by lock line 102, feet 546a, 546b may raise barbells 520 against spring 530, as shown in FIG. 18A. This may draw barbells 520 up along sloping surface 512 which unwedges barbells 520 from against stud 131. In this position, stud 131 is free to move. Thus, when lock line 102 is tensioned to raise or lift harness 540, lock 500 is in an unlocked position wherein stud 131 is free to move actuation mechanism 113 and therefore distal elements 120 to any desired position. Releasing tension in lock line 102 may, on the other hand, transition the lock 500 to a locked position, as shown in FIG. 18B. Thus, by releasing the upwards force on barbells 520 by feet 546a, 546b, spring 530 forces barbells 520 downwards and wedges barbells 520 between a sloping surface 512 and stud 131. This restricts motion of stud 131, which in turn locks actuation mechanism 113 and therefore distal elements 120 in place. In addition, stud 131 may include one or more grooves or indentations 137 which may receive shaft 524 of each barbell 520. This may provide more rapid and positive locking by causing barbells 520 to settle in a definite position, increase the stability of lock 500 by further preventing movement of barbells 520, as well as tangible indication to the user that each barbell 520 has reached a locking position. In addition, grooves 137 may be used to indicate the relative position of distal elements 120, particularly the distance between distal elements 120. For example, each groove 137 may be positioned to correspond with a 0.5- or 1.0-mm decrease in distance between distal elements 120. As stud 131 is moved, barbells 520 may contact grooves 137, and by counting the number of grooves 137 that are felt as stud 131 is moved, the user can determine the distance between distal elements 120 and can provide the desired degree of coaptation based upon leaflet thickness, geometry, spacing, blood flow dynamics and other factors. Thus, grooves 137 may provide tactile feedback to the user.

Lock 500 allows fixation device 112 to remain in an unlocked position when attached to delivery system 1000 during grasping and repositioning and then maintain a locked position when left behind as an implant. It may be appreciated, however, that lock 500 may be repeatedly locked and unlocked throughout the placement of the fixation device 112 if desired. Once the final placement is determined, lock line 102 may be removed, and fixation device 112 may be left behind.

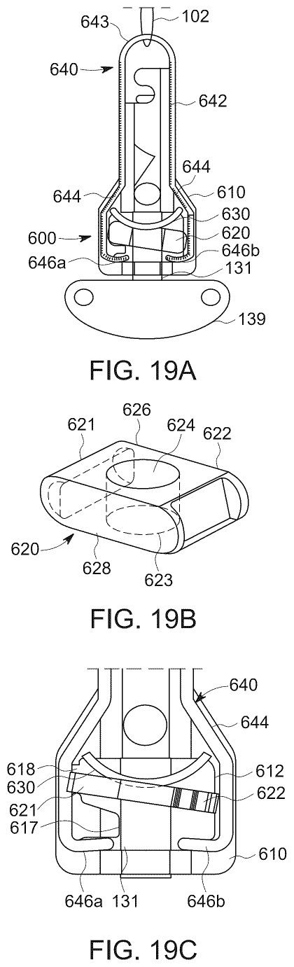

FIGS. 19A-19C depict a lock 600 according to another embodiment of the present disclosure that may be incorporated into a fixation device, such as fixation device 112, for example. In this embodiment, lock 600 also includes a housing 610, a wedging element 620, a biasing element or spring 630, and a release harness 640. Release harness 640 may be the same as release harness 540. However, instead of sloping surfaces 512 as present in the example of lock 500, housing 610 may include sidewalls 612, which may be parallel, and may include a finger or protrusion 617 extending from one of sidewalls 612 toward stud 131, as best shown in FIG. 19C. Such finger 617 may slope in a distal direction and may define a proximal notch 618. Release harness 640 may be the same or similar to release harness 540 in that it may include a proximal portion 642 defining a closed end 643 for receipt and engagement of lock line 102 and a distal portion 644 that includes a pair of inwardly extending feet 646a, 646b. As shown in FIG. 19C, a first foot 646a of release harness 640 may be positioned distal of finger 617 and may be constrained from movement by finger 617, and a second foot 646b may be disposed distal of wedging element 620 and may be free to move into engagement therewith.

Furthermore, wedging element 620 may be in the form of a binding lever or binding plate 620. As shown in FIG. 19B, binding plate 620 may have an oblong shape that may extend lengthwise between a first end 621 and a second end 622 thereof. An opening 624 may be formed between first and second ends 621, 622 and may extend from a top surface or first surface 626 through a bottom surface or second surface 628 of binding plate 620. Top and bottom surfaces 626, 628 may be planar, for example. The intersection of bottom surface 628 with opening 624 forms an edge 623 which may engage stud 131 to secure stud 131 from movement, as described further below. Binding plate 620 may be positioned between sidewalls 612 so that stud 131 passes through opening 626 and so that first end 621 of binding plate 620 is positioned within notch 618 proximal of finger 617, as best shown in FIG. 19C. Thus, finger 617 may be positioned between first end 621 of binding plate 620 and first foot 646a of released harness 620. Also, spring 630 may be positioned proximal to binding plate 620 and provide downward or distal bias thereto. Binding plate 620 and stud 131 may be comprised of any suitable material. In some embodiments, binding plate 620 may have a higher hardness than stud 131. In other embodiments, binding plate 620 may be comprised of a flexible or semi-flexible material. Such flexibility may allow slight movement of stud 131 in the proximal and distal directions, therefore allowing slight movement of distal elements 120 when lock 600 is in the locked position. This may allow fixation device 112 to adjust in response to dynamic cardiac forces.

FIGS. 19A and 19C illustrate binding plate 620 in a locked position or configuration. In this regard, spring 630 pushes binding plate 620 in a distal direction. However, because first end 621 of binding plate 620 is positioned within notch 618, axial movement of first end 621 toward a distal end of housing 610 is prohibited by finger 617 while axial movement of second end 622 of binding plate 620 is permitted. Thus, when lock line 102 is tensioned so as to transition lock to an unlocked position or configuration, finger 617 obstructs first end 621 from axial movement and creates a lever or pivoting-type movement of binding plate 620. Moreover, finger 617 obstructs foot 646a of release harness 620 from axial movement resulting in a side-to-side pivoting of release harness 640 upon tension of lock line 102. This pivoting movement correspondingly results in second foot 646b of release harness 640 moving proximally and controlling movement of second end 622 of binding plate 620. As such, when an upwards force is applied to harness 640 by lock line 102, second foot 646b of release harness 640 raises second end 622 of plate 620 against spring 630 so that top and bottom surfaces 626, 626 of binding plate 620 move toward a perpendicular orientation or close to a perpendicular orientation to stud 131. This aligns opening 624 with stud 131 and disengages edge 623 from stud 131 allowing free movement of stud 131 in the proximal-distal direction. As such, binding plate 620 need not necessarily reach the perpendicular orientation to disengage edge 623 from stud 131 for free movement, as long as edge 623 disengages, stud 131 is free to move. Thus, in this state, lock 600 is unlocked wherein stud 131 is free to move actuation mechanism 113 and therefore distal elements 120 to any desired position.

Releasing tension of lock line 102 and correspondingly on harness 640 transitions lock 600 back to the locked position. By releasing the upwards force on second end 622 of binding plate 620, spring 630 forces second end 622 of biding plate 620 downwards, which misaligns opening 624 relative to stud 131, and correspondingly wedges edge 623 of binding plate 620 against stud 131, as best shown in FIG. 19C. This arrests movement of stud 131, which in turn locks actuation mechanism 113 and therefore distal elements 120 in place. Thus, binding plate 620 is at least partially disposed about stud 131 and is moveable between a first position in which binding plate 620 engages stud 131 to arrest its axial movement, and a second position in which binding plate 620 is disengaged from stud 131 allowing stud 131 to move in the axial direction. In some examples, engagement of binding plate 620 with stud 131 may arrest movement in both a proximal and distal direction. However, in other examples, binding plate 620 may arrest movement of stud 131 in only one axial direction (e.g., the distal direction) so as to form a one-way lock which may allow stud to move in the other direction (e.g., proximal direction) allowing distal elements 120 to continue to be closed (i.e. moved toward each other) while prohibiting distal elements 120 from being opened (i.e., moved away from each other).

It may be appreciated that binding plate 620 may have any suitable form to function as described above. For example, plate 620 may have a variety of shapes with or without planar surfaces 626, 628 and/or the opening 624 may be of a variety of shapes and positioned in a variety of locations, to name a few. For example, binding plate 620 may not have a through-hole, like that of opening 624, but may rather have a notch such that binding plate 620 does not encircle stud 131 but rather partially surrounds it. Further, it may be appreciated that any number of binding plates 620 may be present. Each binding plate 620, in this regard, may provide an additional binding location which may enhance lock performance.

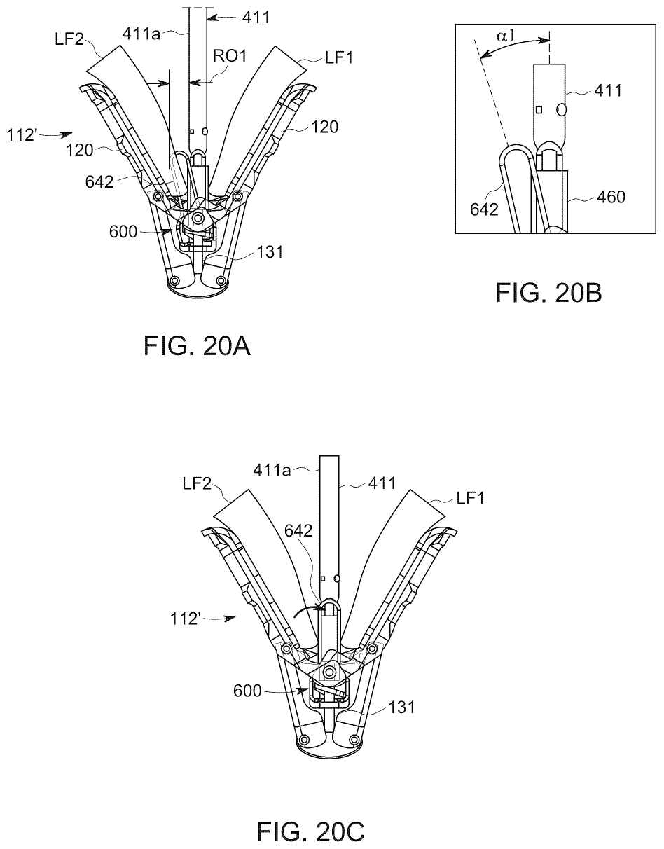

FIGS. 20A-20C illustrate a fixation device 112′ in use to capture tissue, such as opposing leaflets LF1, LF2 of a heart valve, such as a mitral valve MV or tricuspid valve TV, for example. Fixation device 112′ includes lock 600. FIGS. 20A and 20B depict fixation device 112′ in an unlocked configuration, and FIG. 20C depicts fixation device 112′ in a locked configuration. As shown in FIGS. 20A and 20B, and as described above, when release harness 640 is tensioned to the unlocked configuration, such as via lock line 102, second foot 646b moves proximally to engage binding plate 620 so as to disengage binding plate 620 from stud 131, while first foot 646a remains substantially stationary beneath finger 617 of housing 610. Thus, as lock 600 is unlocked, release harness 640 moves proximal and also rotates or tilts outwardly toward a distal element 120 that is at the same side of fixation device 112′ as first foot 646a. The result is that proximal end 642 of release harness 640 moves radially away from delivery system shaft 411 and coupling member 460. In other words, release harness 640, when in the unlocked configuration, forms an excursion or runout RO1 into the area where tissue. such as leaflet L2, is captured between a distal and proximal element 120, 140. As shown in FIG. 20A, this runout RO1 can be measured between a lateral periphery 411a of delivery shaft 411 and the furthest most edge of proximal portion 642 of release harness 640. It should be noted that shaft 411 and coupling member 460 are depicted, but any of the aforementioned shafts 111, 311 and coupling members 160, 360 may be utilized. As an example, at a nominal lock line load (e.g., 1.25 lbf to 3.0 lbf), the runout RO1 of lock 600 can be upwards of approximately 0.033 in. (0.838 mm) to 0.036 in. (0.914 mm). As another example, as shown in FIG. 20B, harness 640 can form a tilt angle α1 as measured from a longitudinal axis of shaft 411 and a center of proximal portion 642 of harness 640 which may be upwards of 12 degrees when in the unlocked configuration. On the other hand, when lock 600 is in the locked configuration, the runout RO1 is minimal and proximal portion 642 and tilt angle α1 is approximately 0 degrees, as shown in FIG. 20C.

The runout RO1 of release harness 640 when in the unlocked configuration can limit the degree to which distal elements 120 can be closed prior to initiating locking. For example, distal elements 120 in a TEER procedure may form an angle between them of about 10 to 30 degrees when leaflets LF1, LF2 are captured, distal elements 120 are fully closed, and fixation device 112′ is released from shaft 411. However, if a user attempts to close distal elements to 10 to 30 degrees of closure prior to initiating locking, leaflet LF1 and/or leaflet LF2 may impinge on release harness 640 prior to reaching such degree of closure which can cause difficulties in establishing the locked configuration. Thus, to avoid such difficulties, locking may be established by a user at a threshold locking angle in which impingement is avoided. In other words, the threshold locking angle is referred to herein as the minimum angle between fixation elements 120 that lock 600 can consistently be engaged without interference from tissue irrespective of tissue variations from patient to patient. Such threshold locking angle may be 60 degrees or greater, for example. In other words, if distal elements 120 are closed to an angle less than the threshold angle while lock 600 is in the unlocked configuration, leaflet LF1 may block harness 640 from returning to its centered, locked position, for example. In such circumstance, fixation device 112′ may inadvertently open, such as up to an additional 10 degrees, before the obstruction caused by leaflet L1 is cleared and lock 600 can engage stud 131. Such inadvertent opening can undermine the repair.

Additionally, it may be desirable to vary the degree of closure during a procedure until the desired reduction in regurgitation is achieved without overly restricting blood flow over the valve into the ventricle. In other words, decreasing the closure angle of distal elements 120 may further decrease regurgitation but may correspondingly restrict desirable blood flow to the ventricle. Thus, it may be desirable to unlock lock 600 to expand distal elements 120 whether it be for testing other closure angles or to attempt regrasping. However, once distal elements 120 are closed to a narrower degree relative to the threshold locking angle, unlocking may be restricted or impinged by leaflet L2 potentially prohibiting lock 600 from unlocking.

Furthermore, harness 640 can be inadvertently pushed into the unlocked configuration thereby inadvertently unlocking lock 600. For example, in circumstances where leaflet LF1 is relatively thick, and leaflet L2 is relatively thin, the thick leaflet LF1 may push proximal portion 642 of release harness 640 towards the thin leaflet LF2 during closure which can cause lock 600 to unlock potentially disrupting the valve repair.

### G6 Locking Mechanism

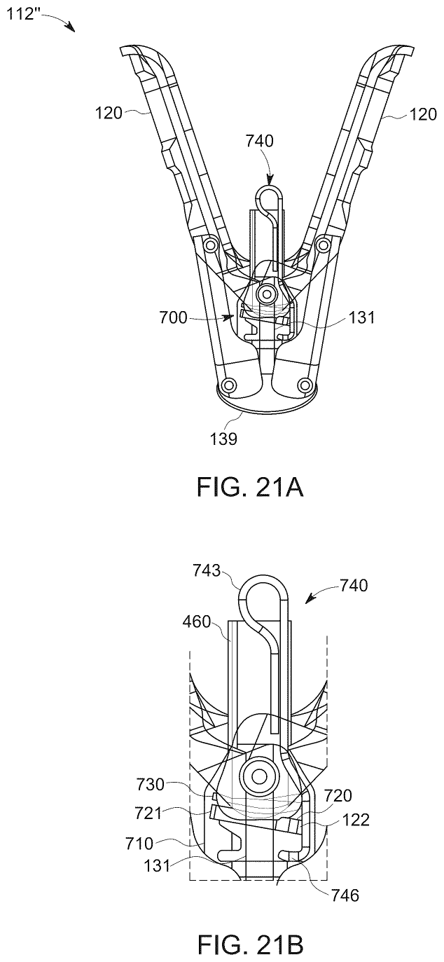

FIGS. 21A and 21B depict a fixation device 112″ which includes a lock 700 according to another embodiment of the present disclosure. Lock 700 generally includes a housing 710, a binding plate 720 (or wedging element), a biasing element 730, and a release harness 740. Housing 710 and binding plate 720 are similar to housing 610 and binding plate 620 and are therefore accorded corresponding reference numerals in the 700-series of numbers. However, release harness 740 differs substantially in that it is a single-sided harness and is configured to limit runout while providing sufficient strength and fatigue resistance, as described below. Additionally, biasing element 730 is constructed for increased reliability as compared to biasing element 630. Between fabrication, quality control testing, and use during a procedure, biasing element 630 may undergo multiple cycles of loading and unloading which can fatigue biasing element 630 leading to permanent deformation potentially leading to a weakened engagement force and an increased rate of inadvertent fixation device opening. Biasing element 730 is configured to resist such deformation, as described below.

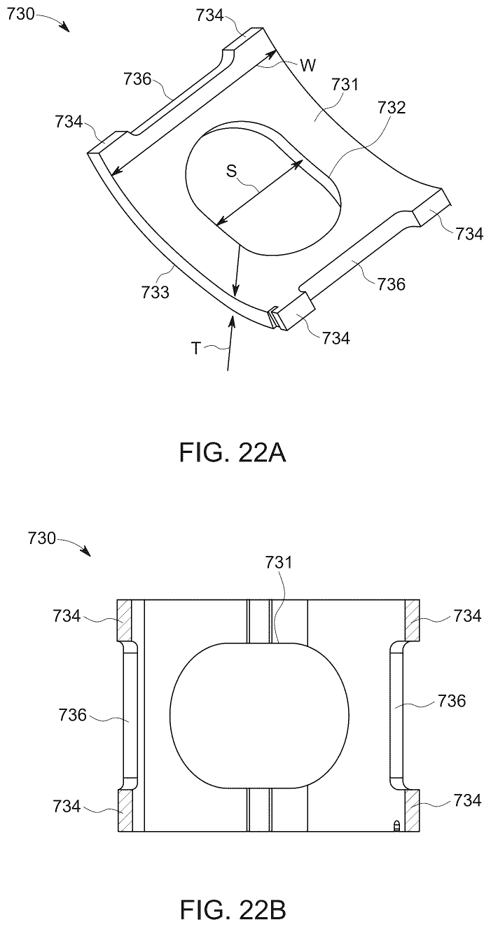

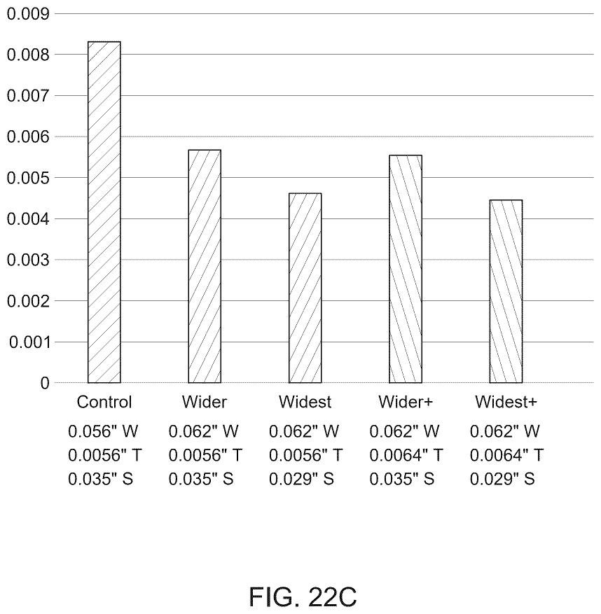

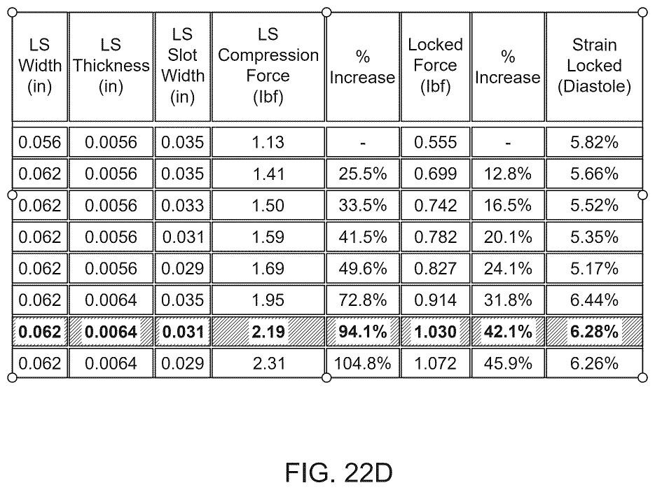

FIGS. 22A and 22B depict biasing element 730 according to one example. Biasing element 730 may be a leaf spring with a concave surface 731 and a convex surface 733 disposed opposite concave surface 731. A thickness T of biasing element 730 is defined between concave and convex surfaces 731, 733. Biasing element 730 defines a width W spanning from edge to edge and a slot 732 extending through concave and convex surfaces 731, 733. Slot 732 defines with a slot width S. Opposing ends of biasing element 730 may each include a pair of legs 734 which define a recess 736 therebetween. Such recesses 736 may receive a portion of housing 710 such that legs 734 bear against housing 710 thereby securing biasing element 730 to housing 710. As shown in FIG. 21B, biasing element 730 may be disposed within housing 710 such that its convex surface 733 faces binding plate 720. However, in some embodiments, biasing element 730 may be oriented such that its concave surface 731 faces binding plate 720. As compared to biasing element 630, biasing element 730 has a greater thickness T, a wider width W, and a narrower slot width S. For example, thickness T of biasing element 730 may be 0.0058 in. (0.1473 mm) to 0.0069 in. (0.175 mm), but preferably 0.0064 in. (0.1626 mm), width W of biasing element 730 may be 0.060 in. (1.524 mm) to 0.062 in. (1.575 mm), and slot width S may be 0.029 in. (0.737 mm) to 0.031 in. (0.7874 mm). On the other hand, biasing element 610 may have a thickness of 0.0056 in. (0.1422 mm), a width of 0.056 in. (1.422 mm), and a slot width of 0.035 in. (0.889 mm).

Additionally, the thickness T and width W of biasing element 730 may have a relationship with a width and thickness of binding plate 720 for enhanced performance. For example, a ratio between a thickness of binding plate 720 and thickness of biasing element 730 may be about 1.74 to 2.24, but preferably 1.95. In another example, a ratio between a width of binding plate 720 and a width of biasing element 730 may be about 0.84 to 0.9, but preferably 0.87.

Such dimensional differences of biasing element 730 have demonstrated unexpectedly significant improved performance and deformation resistance compared to biasing element 630 particularly. For example, FIG. 22C illustrates improvements in deformation resistance of biasing element 730 compared to the control (i.e., biasing element 630). Test samples were tested using an Instron test platform at 5 lbf for 20 cycles at 37 degrees Celsius. The results show a marked improvement of about 35% to 45% resistance in flattening for biasing element 730 compared to the control 630. Additionally, FIG. 22D tabulates results of a finite element analysis of biasing element 730 compared to the control (i.e., biasing element 630) and demonstrates a significant improvement in engagement force which correspondingly improves lock reliability and consistency. For example, as shown, biasing element 730 unexpectedly provides 94% to 105% increase in compression force and a 42% to 46% increase in locked force as compared to biasing element 630. The increase in width W, narrowing of slot width S, and increase in thickness T increases the locking force and balances the strain for enhanced performance and reliability of lock 700. Through this testing it was learned that simply increasing the thickness of the biasing element 730 as compared to biasing element 630 increased strain significantly making it more prone to deformation, but that by widening biasing element 730 and narrowing slot width S, strain was reduced significantly and unexpectedly in what appears to be due to a beneficial effect on stress concentrations. In other words, thickening biasing element 730 resulted in an expected increase in strain. However, widening biasing element 730 and narrowing slot width S had a more dramatic effect on strain reduction than expected as it was anticipated that such dimensional changes would not have been able to overcome the increased strain. The superior performance of biasing element 730 results from a balance between material thickness, width, and slot geometry that optimizes both elastic deformation capacity and spring force. The 0.0064 in. thickness creates a cross-sectional moment of inertia that, when combined with the 0.060-0.062 in. width and 0.029-0.031 in. slot width, generates an ideal stress-strain curve. Finite element analysis revealed that this dimensional relationship creates stress distribution patterns that prevent plastic deformation at typical operating loads. The narrow slot width compared to biasing element 630 creates beneficial stress concentration zones that counterintuitively strengthen rather than weaken the overall structure by distributing strain more evenly across the entire element.

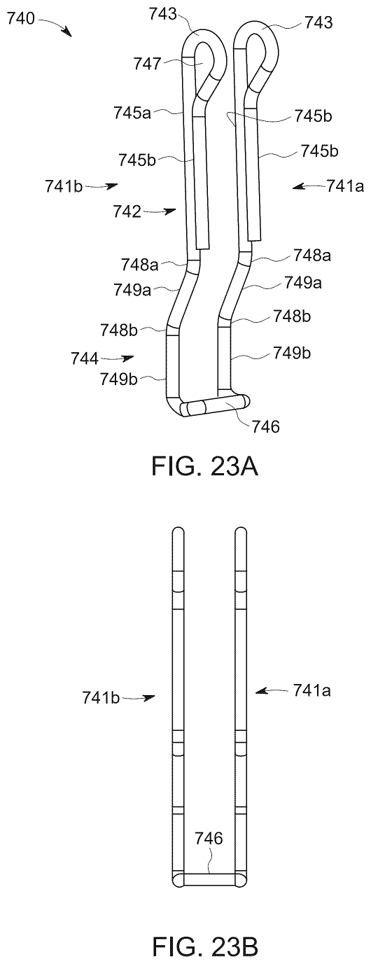

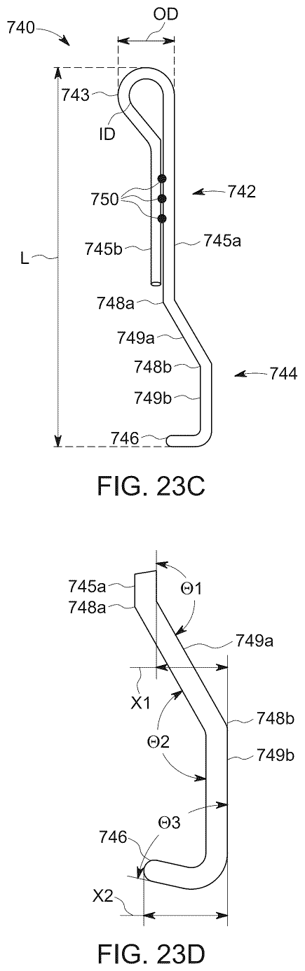

FIGS. 23A-23D depict release harness 740 according to one example. Harness 740 may be in the form of a ridged wire or rod that may extend proximally from between base 139 and coupling member 460 toward a proximal end of fixation device 112″. Release harness 740 may have a first section or front section 741a and a second section or rear section 741b which, when coupled to fixation device 112″, are respectively disposed at a front and rear of stud 131.

Harness 740 may also include a first end portion or proximal portion 742 and a second end portion or distal portion 744. In the embodiment depicted, proximal portion 742 of each section 741a, 741b of harness 740 may include a loop or closed end 743 which may define a proximal terminal end of harness 740. However, in some embodiments, only one of sections 741a, 741b may include a loop 743. In the embodiment depicted, loop 743 of each section 741a, 741b is formed by the wire or rod that comprises harness 740 and is a closed loop that defines an eyelet or opening 747. In this regard, proximal portion 742 of each section 741a, 741b may include a first straight segment 745a and a second straight segment 745b with loop 743 being disposed therebetween. Second straight segment 745b may define a terminal end of the wire comprising harness 745b, as shown in FIGS. 23A and 23C. First and second straight segments 745a, 745b may be connected so as to ensure the integrity of loop 743 and harness 740. In other words, the connection of first and second straight segments 745a, 745b helps resist fracturing of harness 740 under repeated loading cycles (i.e., repeated locking and unlocking of lock 700). Such connection may be achieved via various welding techniques, including spot welding, seam welding, or wobble welding, such a zig-zag wobble welding, for example. In some embodiments, the welds may be configured as partial depth welds that do not fully penetrate the segments, thereby preventing melted material from extending inboard where it could contact adjacent components and generate friction. This shallow weld approach helps ensure the locking mechanism behaves repeatably under different loads. In one specific example depicted in FIG. 23C, three spot welds 750 may connect first and second straight segments 745a, 745b. However, more spot welds 750 may be utilized, such as four or five spot welds 750, for example, which can provide additional strength and improved stress distribution as desired. In some embodiments, five partial depth spot welds may be utilized to maximize strength and stress distribution across the connection while maintaining loop integrity and preventing interference with adjacent components. The placement of three to five welds between segments 745a and 745b creates a stress distribution zone that prevents localized strain concentration.

Additionally, the dimensions of each loop 743 are minimized to limit runout as compared to harness 640. For example, each loop 743 may have an inner diameter or inner dimension ID sufficiently large to allow the passage of lock line 102 without restricting its passage and without generating substantial friction that can undermine the lock when lock line 102 is withdrawn from loops 743, and an outer diameter or outer dimension OD that minimizes runout. For example, the inner diameter ID may be 0.015 in. (0.381 mm) to 0.025 in. (0.635 mm). Further, outer diameter OD may be 0.037 in. (0.940 mm) to 0.041 (1.041 mm). Such outer diameter OD may be smaller than a cross-sectional dimension of coupling member 460, as shown in FIG. 21B. Thus, when leaflets, such as leaflets LF1 and LF2 of FIG. 20A, are compressed toward the center of fixation device 112″, their engagement with release harness 740 is minimal as coupling member 460 may provide a barrier to such engagement. Additionally, connecting first and second straight segment 745a, 745b also has the added benefit of reducing the profile of proximal portion 742 such that a maximum lateral width of proximal portion 742 is the maximum width, or outer diameter OD, of loop 743. Thus, as shown in FIG. 23C, first and second straight segments 745a, 745b have an even narrower lateral profile than loops 743 which further minimized the profile of harness 740.

Distal portion 744 of harness 740 may include a single foot or inward extension 746 that extends inwardly. In this regard, harness 740 may be referred to herein as a single-sided harness (“SSH”) or single-foot harness. In the embodiment depicted, foot 746 may be bent inwardly, such as by bending the wire that may form harness 740. In other words, first straight segment 745a of proximal portion 742 may define a longitudinal axis, and foot 746 may be bent inwardly toward such longitudinal axis, as shown in FIGS. 23C and 23D. However, in other embodiments, foot 746 may be separately connected to harness 740 rather than integral therewith. Additionally, as illustrated in FIGS. 23A and 23B, foot 746 may extend between and connect first and second sections 741a, 741b. Thus, when harness 740 is coupled to fixation device 112″, foot 746 may be positioned at one side of stud 131 and may extend front to rear through housing 710, as shown in FIG. 21B. As shown, foot 746 extends continuously from first section 741a to second section 741b. In some embodiments, foot 746 may be discontinuous (i.e., interrupted) between first and second sections 741a and 74b. In such embodiments, foot 746 may nonetheless still be considered a single foot.

Furthermore, distal portion 744 may flare outwardly relative to proximal portion 742, as shown in FIG. 23C such that distal portion forms a side of harness 740 which, as shown, is a single side. Such outward flare offsets foot 746 relative to proximal portion 742 such that foot may be disposed distal a second end 722 of binding plate 720 while proximal portion 742 extends along coupling member 460 and delivery system shaft 411 when harness is coupled to fixation device 112″, as illustrated in FIG. 21B. In other words, proximal portion 742 may define an axis of harness 740, and foot 746 may be disposed to one side of such axis. Since harness 740 has only a single foot 746, harness 740 is considered a SSH. This is in contrast to harness 640 which is a DSH as it has feet 646a, 646b disposed at opposite sides of an axis defined by proximal portion 642.

Distal portion 744 of each of front and rear sections 741a, 741b may have a series of bends 748a, 748b and straight segments 749a, 749b. Such bends 748a, 748b may be configured to offset foot 746 to the one side of harness 740, as mentioned above. For example, as shown, distal portion 744 of each section 741a, 741b may include first and second bends 748a, 748b and first and second straight segments 749a, 749b. As shown in FIG. 23D, first bend 748a may be connected to one end of first straight segment 745a of proximal portion 742 and one end of first straight segment 749a of distal portion 744. Additionally, second bend 748b may be connected to another end of first straight segment 749a of distal portion 744 and one end of second straight segment 749b of distal portion 744 while foot 746 may be connected to the other end of second straight segment 749b. Additionally, first straight segment 749a of distal portion 744 may be angled relative to first straight segment 745a of proximal portion 742 by a first angle Θ1 and relative to second straight segment 749b of distal portion 744 by a second angle Θ2. Such angles Θ1, Θ2 may be offsetting such that second straight segment 749b is oriented parallel to first straight segment 745b of proximal portion 742. For example, first angle Θ1 may be 150 degrees in a first rotational direction (±/−), and second angle Θ2 may be 150 degrees in an opposite rotational direction (±/−). Although in the depicted embodiment each section 741a, 741b includes first and second bends 748a, 748b and first and second straight segments 745a, 745b, in other embodiments, more or less bends and straight segments may be provided. Alternatively, such bends 748a, 748b and straight segments 749a, 749b may instead be one bent section that curves in an arc from proximal portion 742 to foot 746.

Foot 746 may be angled at a third angle Θ3 relative to second straight segment 749b of distal portion 744. Third angle Θ3 may be an acute angle, such as 80 to 85 degrees, for example, but preferably 80 degrees. As discussed below, such third angle Θ3 provides numerous benefits. The foot angle Θ3 of 80-85 degrees represents a compromise between force transmission efficiency and deformation resistance. At this angle, the vector components of the tensile force applied through lock line 102 are optimally distributed between the axial force needed to displace binding plate 720 and the lateral force component that contributes to runout. When foot angle Θ3 decreases below 80 degrees, the enhanced mechanical advantage can create excessive force amplification at the binding plate interface, leading to premature disengagement. Conversely, angles exceeding 85 degrees can result in suboptimal force transmission requiring excessive input tension to achieve displacement. Foot 746 may be moveable proximally-distally within housing 710 and may be positioned distal to a second end 722 of binding plate 720 such that pulling up on harness 740 moves foot 746 proximally so as to push second end 722 of binding plate 720 against the bias of biasing element 730 and pivot binding plate 720 to a perpendicular orientation or near perpendicular orientation sufficient to disengage binding plate 720 from stud 131. Thus, lock 700, has a locked configuration and an unlocked configuration. The locked configuration is shown in FIGS. 21A and 21B and, in the locked configuration, binding plate 720 is oriented at an oblique angle relative to stud 131 such that binding plate 720 wedges against stud 131 arresting its axial movement.

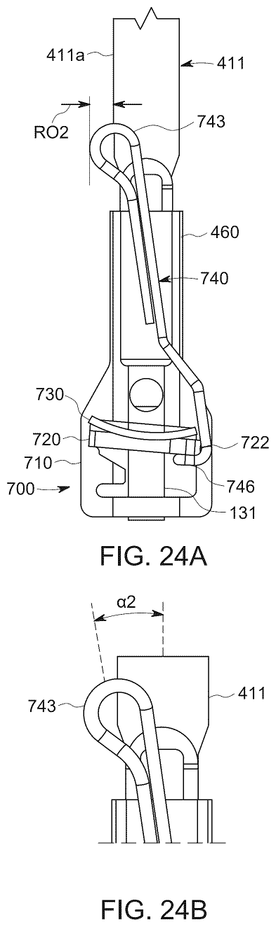

The unlocked configuration is shown in FIGS. 24A and 24B and, in the unlocked configuration, foot 746 bears against second end 722 of binding plate 720 such that it resists the bias of biasing element 730 and positions it at an orientation that allows stud 131 to move freely in an axial direction. Thus, when release harness 740 is tensioned to the unlocked configuration, such as via lock line 102, foot 746 moves proximally to engage binding plate 720 so as to disengage binding plate 720 from stud 131. Thus, as lock 700 is unlocked, release harness 740 moves proximally and also rotates or tilts outwardly toward a distal element 120 that is at an opposite side of foot 746. Although release harness 740 tilts outwardly to form an excursion or runout RO2, such runout RO2 is significantly lessened relative to that of release harness 640 so as to alleviate the potential issues described above with respect to harness 640. As shown in FIG. 24A, this runout RO2 can be measured between a lateral periphery 411a of delivery shaft 411 and the furthest most edge of loop 743 of proximal portion 742. It should be noted that shaft 411 and coupling member 460 are depicted, but any of the aforementioned shafts 111, 311 and coupling members 160, 360 may be utilized. As an example, at a nominal lock line load (e.g., 1.25 lbf to 3 lbf), the runout RO2 of lock 700 can be upwards of about 0.011 in. (0.279 mm) to 0.018 in. (0.457 mm). Thus, the runout RO2 is reduced to approximately one-third that of runout RO1. As another example, as shown in FIG. 20B, harness 740 can form a tilt angle α2 as measured from a longitudinal axis of shaft 411 and a center of loop 743 of harness 740 which may be upwards of 6 degrees when in the unlocked configuration as compared to the approximately 12-degree tilt angle α1 of harness 640. In other words, harness 740 may be moveable from a first orientation to a second orientation, the second orientation being 6 degrees or less than the first orientation. In the first orientation, first straight segment 745a of proximal portion 742 is parallel to a longitudinal axis of stud 131. Thus, in the second orientation, first straight segment 745a may be oriented up to 6 degrees from the longitudinal axis of stud 131.

This significant reduction in runout between harness 740 and harness 640 reduces the threshold locking angle of fixation device 112″ to 30 degrees or less, at which locking can be initiated thereby reducing the likelihood of lock 700 will not engage and allowing the user to more deliberately modify the repair. Additionally, the risk of inadvertent unlocking due to a thick/thin leaflet configuration is significantly reduced or eliminated. The reduced runout RO2 can be attributable at least to the low profile of proximal portion 742 of harness 740 and the significantly reduced tilt angle α2 which is at least a function of the single-sided construction of release harness 740. In this regard, the single-sided construction limits the pivoting action of release harness 740 such that movement of harness is primarily in the axial direction. Further, as mentioned above, the foot angle Θ3 relative to second straight segment 749b, and correspondingly, first straight segment 745a of proximal portion 742, is 80 to 85 degrees, and preferably 80 degrees. This foot angle Θ3 has been found to provide optimal performance. For example, angles Θ3 greater than 85 degrees can deform to such a degree that performance is compromised. For example, angles Θ3 greater than 85 degrees have been found to become more easily wedged between binding plate 720 and housing 710 thereby increasing drag. Additionally, foot angles Θ3 greater than 85 degrees have been found to increase the length L of harness and correspondingly increases the runout RO2. Thus, the foot angle also Θ3 limits runout of harness 740 to so that the benefits mentioned can be realized. It has also been found, surprisingly, that foot angles Θ3 less than 80 degrees can make it too easy to unlock lock 700 running the risk of inadvertent unlocking when the user pulls lock line 102 out from loops 743. For example, tests were performed with the foot angle Θ3 at 45 and 60 degrees. It was found that lower foot angles Θ3 such as these create a more efficient unlock mechanism such that unlocking became too easy and removing lock line 102 from eyelets 743 unintentionally unlocked lock 700.

One effect of implementing an SSH, like harness 740, versus an DSH, like harness 640, is that tension from lock line 102 is concentrated in the single foot 746 rather than distributed to two feet, like feet 646a, 646b, which may result in harness 740 being more vulnerable to deformation. It has been found that a wire diameter of 0.0075 in. (0.1905 mm) to 0.008 in. (0.203 mm), and preferably 0.008 in., provides a balance between strength and operability as wire diameters less than 0.0075 in. were found to cause severe stretching resulting in an increase in foot angle Θ3 under load making foot 746 more susceptible to becoming wedged between binding plate 720 and housing 710. For comparison, the wire diameter of harness is 0.007 in. (0.178 mm). On the other hand, wire diameters greater than 0.008 in. have been found to increase the runout RO2 of harness 740 and reduce the inner diameter ID of loop 743 potentially undermining the benefits conferred by harness 740.

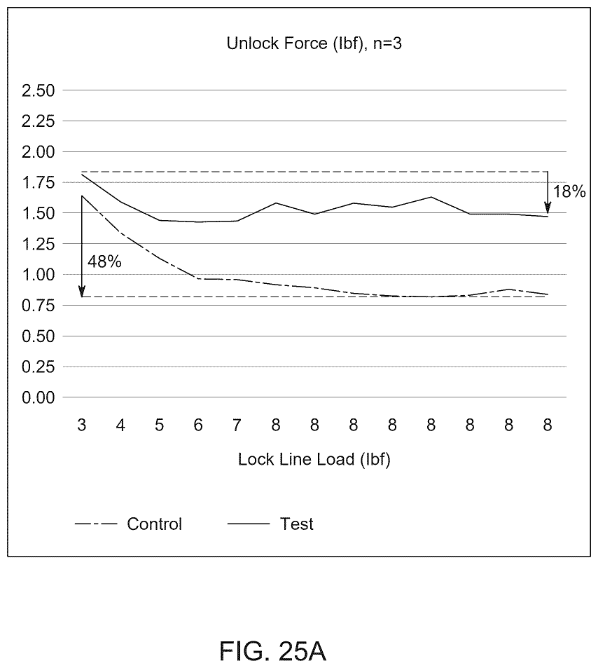

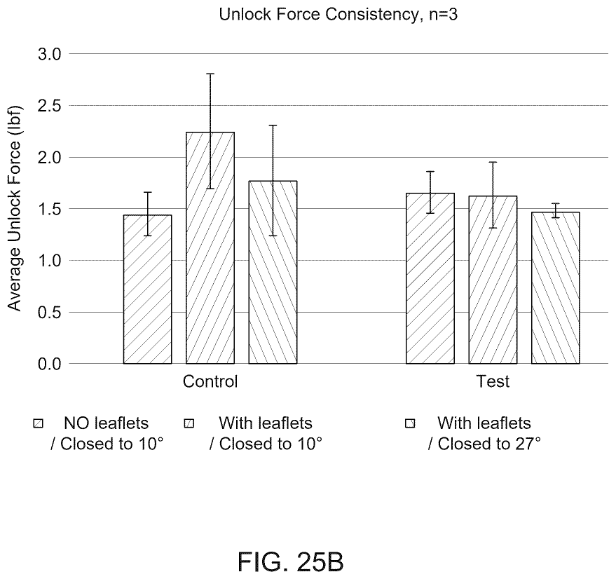

FIGS. 25A and 25B further illustrate the unexpectedly enhanced performance of lock 700. For example, as shown in FIG. 25A, three samples of a control (i.e., fixation devices with lock 600) were compared to three test samples (i.e., fixation devices with lock 700). Such samples were subjected to increasing lock line tension through repeated cycles of locking and unlocking, and the unlock force was measured. In this regard, this test method evaluates the locking force of the lock by overcoming it with an unlock operation where the force to unlock is measured. It would have been expected that, with repeated cycles of loading and unloading, release harnesses 640 and 740 would deform resulting in a decrease in unlocking force. Additionally, it would have been expected that DSH 640 with load distributed to its two feet 646a, 646b would outperform SSH 740, which concentrates load in its single foot 746. In other words, it would have been expected for SSH 740 to have greater deformation and thereby a greater decrease in unlocking force over repeated cycles than DSH 640. However, the opposite occurred in a surprising result. Harness 740 saw a reduction in 18% in unlocking force, while harness 640 saw a 48% reduction in unlocking force demonstrating that harness 740 is less sensitive to harness deformation than harness 640.

Furthermore, FIG. 25B demonstrates that lock 700 is also less sensitive to leaflet interactions and provides more consistent unlocking forces. In this example, three samples of a control (i.e., fixation devices with lock 600) were compared to three test samples (i.e., fixation devices with lock 700). Such samples were closed to 10 degrees without leaflets, 10 degrees with leaflets, and 27 degrees with leaflets. The presence of leaflets can increase the force for unlocking the lock. This test demonstrated that the unlocking force did indeed increase for lock 600 due to interactions with leaflets, but that the unlocking force for lock 700 remained relatively constant even in the presence of leaflets and at tight angles of closure (e.g., 10 degrees of closure). Thus, despite being more prone to deformation, harness 740 of lock 700 has been found to be unexpectedly robust and more stable when exposed to cyclic loads across a range of increasing loads as compared to harness 640. Lock 700 demonstrates a system where dimensional relationships between various components contribute to the enhancement overall performance. Single-sided harness configuration including the foot angle Θ3, the biasing element dimensions, and the corresponding spool diameter (discussed below) can each provide benefits individually, while also offering enhanced performance when implemented together in various combinations. Testing showed that these features provide flexibility in design implementation, as the performance advantages may be realized through different combinations of these features depending on the specific application requirements.

While the above-described nonlimiting examples of fixation device 112 may utilize a push-to-open, pull-to-close mechanism for opening and closing distal elements 120, it should be understood that a pull-to-open, push-to-close mechanism may alternatively be provided. For example, distal elements 120 may be coupled at their proximal ends to stud 131 rather than to coupling member 160, and legs 130 may be coupled at their proximal ends to coupling member 160 rather than to stud 131. In this example, when stud 131 is pushed distally relative to coupling member 160, distal elements 120 may close, while pulling on stud 131 proximally toward coupling member 160 may open distal elements 120. Regardless, the aforementioned lock examples may be configured to arrest stud to lock distal elements 120 in the desired position, as described.

While the SSH 740 of lock 700 provides significant advantages as described above, further testing and analysis has revealed specific challenges associated with its implementation. The primary challenge involves side loading that can occur during operation of lock 700. Due to the inherent asymmetry of the single-sided configuration, harness 740 applies force that is not purely axial to binding plate 720. Without a counterbalancing foot on the opposite side, as in the case of DSH 540, there is no counterforce to restrict binding plate motion, which can result in undesirable lateral movement of binding plate 720.

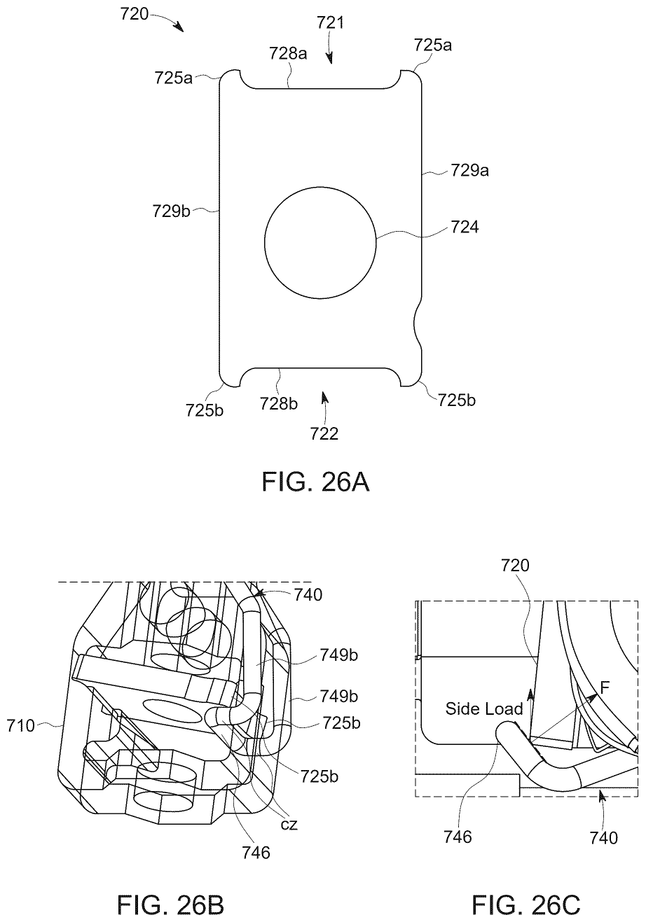

As shown in FIG. 26A, binding plate 720 extends lengthwise between a first end 721 and a second end 722. First end 721 defines a first end edge 728a and a pair of ears (or projections) 725a extending from first end edge 728a at opposite sides of plate 720 such that first end edge 728a is recessed relative to ears 725a. Second end 722 defines a second end edge 728b and a pair of ears (or projections) 725b extending from second end edge 728b such that second end edge 728b is recessed relative to the pair of ears 725b. Binding plate 720 has a first side edge 729a and a second side edge 729b extending along the length of the binding plate. Binding plate 720 has a top surface 723a and a bottom surface 723b. A through hole 724 extends through the top and bottom surfaces for receipt of stud 131.

The SSH configuration of lock 700, while more efficient than the DSH configuration of lock 600, creates force vectors that can contribute to side loading. As shown in FIG. 26B, the contact between foot 746 of harness 740 and second end 722 of binding plate 720 occurs primarily at ears 725b of binding plate 720 and at opposite sides of foot 746 of release harness 740. These contact zones (CZ) are positioned such that a component of the applied force F generates a side load rather than a purely axial force, as shown in FIG. 26C. This side load component of the force vector F creates lateral movement rather than purely axial displacement of binding plate 720.

Several factors contribute to the development of side loading during operation. First, the ear features 725b at the second end 722 of binding plate 720 create points where foot 746 can catch or apply non-axial force. In some instances, foot 746 can become trapped within one or both ears 725b, further exacerbating the side loading effect. Second, biasing element 730, being stiffer than biasing element 630, necessitates more lifting force to compress. This increased force demand amplifies any non-axial force components, which can make side loading more pronounced during operation. Additionally, the routing of lock line 102 diagonally across catheter shaft 411 contributes to side loading by creating lateral force components rather than purely axial tension. Testing has also revealed that harness foot 746 can experience deformation under load, which further alters its contact point and force vector, potentially increasing side loading through cyclic operation of lock 700.

Such side loading can impact lock performance. When side loading occurs, binding plate 720 can press against or bind up against an inner surface of housing 710 or other structures within housing 710. This creates variable friction and resistance when attempting to open fixation device, such as fixation device 112, leading to inconsistent performance. As binding plate 720 experiences side loading, it may freely translate into contact with stud 131, particularly when locking device 700 is unlocked. This translation can increase resistance during opening operations, especially when unlocking occurs at higher lock line loads. Side loading can also cause foot 746 to become trapped or wedged within ears 725b of binding plate 720. This wedging effect creates a mechanical interlock that may increase opening resistance and potentially prevents proper device function. Side loading creates variability in how much force is needed to open the device, resulting in unpredictable performance.

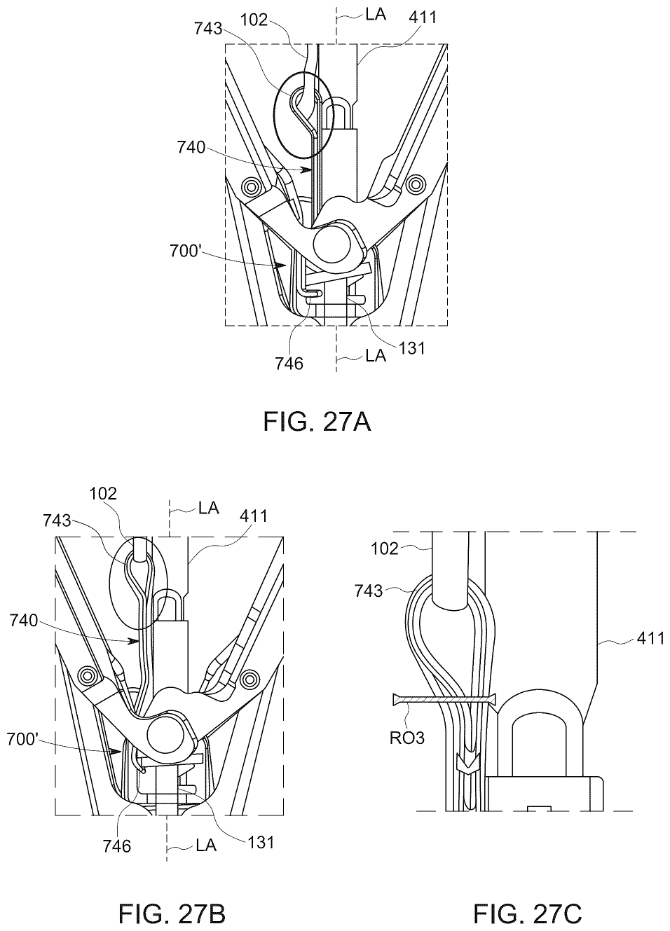

Several modifications to release harness 740 have been developed which can address side loading and wedging of binding plate 740 as described above. One exemplary approach involves modifying the lock line routing to create a more favorable force vector. In this exemplary configuration, as shown in FIGS. 27A-27C, lock line 102 routing is changed relative to that described with respect to FIGS. 24A and 24B. In this regard, lock line 102 extends through release harness 740, such as through eyelet 743, at the same side of a longitudinal axis LA of a catheter shaft 411 and/or fixation device stud 131 as foot 746 which allows lock line 102 to pull from the same side of catheter shaft 411 as foot 746 instead of diagonally across the longitudinal axis LA. FIG. 27A shows the lock 700′ in the locked configuration with lock line 102 extending through eyelet 743 of release harness 740 at the same side of stud 131 and catheter shaft 411 as that of foot 746. FIG. 27B shows the lock 700′ in the unlocked configuration with lock line 102 tensioning the eyelet 743 of release harness 740.

As shown in FIG. 27C, the runout RO3 of release harness 740 is negligible as compared to the locked configuration. In this regard, the runout RO3 is effectively the dimension of the eyelet 743 as the travel of pull via lock line 102 is purely in the axial direction. In the example depicted, the dimension of the eyelet 743 and, therefore, the runout RO3 is 0.0362 in. This lifting-side lock line routing creates a more direct, purely vertical pulling force on harness 740, reducing opening force even at higher loads by eliminating the lateral force components that contribute to side loading. This approach fundamentally changes the direction of applied force to ensure more axial loading on binding plate 720.

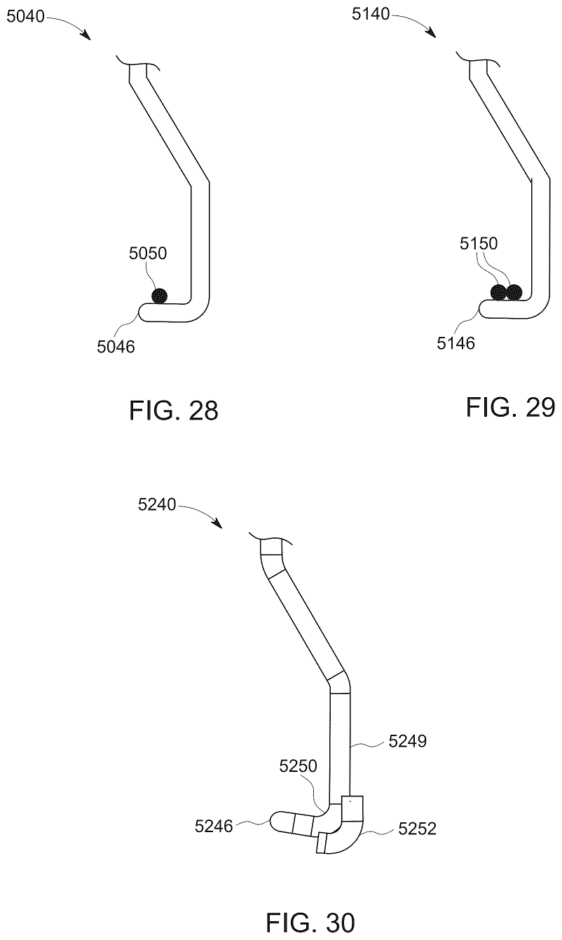

FIG. 28 depicts a release harness 5040 according to another example. Release harness 5040 mitigates sideloading through a modified contact geometry via the addition of contact features to a harness foot 5046. In this example, release harness 5040 is the same as release harness 740 except that the foot 5046 has a single bump 5050 extending from it in an upward or proximal direction. Bump 5050 may also extend from one side of harness 5040 to the other in the same direction as that of foot 5046.

In another example shown in FIG. 29, a release harness 5140 has a plurality of bumps 5150, such as two bumps 5150, for example, arranged adjacent to each other on a foot 5146.

The bumps 5050, 5150 of harnesses 5040 and 5140 help ensure lock efficiency by avoiding wedging of harness between 5040, 5140 between a binding plate, such as binding plate 720, and a lock housing, such as housing 710. This is achieved by controlling the contact location between harness 5040, 5140 and binding plate 720. The raised bumps 5050, 5150 contact binding plate 720 more centrally rather than at ends of feet 5046, 5146 where wedging could occur, as described above with respect to FIG. 26B, thereby providing enhanced control over the force transmission between components.

FIG. 30 depicts a release harness 5140 according to a further example. Release harness 5140 is the same as that of harness 740 except that the part of the harness that forms foot 5146 is reinforced with extra material 5150 to resist bending. In this regard, a bend 5250 is formed between foot 5246 and straight segment 5429 of harness 5240. Such bend 5250 is susceptible to deformation when foot 5246 engages binding plate 740 under lock line tension. Testing has revealed that foot deformation precedes wedging, suggesting that maintaining the original foot geometry under load would mitigate side loading issues. Alternatively, a starting foot angle less than 80-90 degrees such as 60 degrees may provide favorable performance as deformation to a final angle of 80 degrees correlates with high quality performance. In the example depicted, the wire structure of harness 5240 is strengthened with extra material reinforcement at the bend 5250 between straight segment 5249 and foot 5246 so as to help maintain foot geometry (e.g., foot angle Θ3) under operational loads which in turn helps prevent harness wedging and a more consistent and reliable operation.

It should be noted that any of the aforementioned release harness embodiments (740, 5040, 5140, 5240) may be combined with each other to achieve a cumulative benefit in reducing sideloading effects. For example, the lock line orientation modification shown in FIGS. 27A-27C may be implemented together with the single or double bump features of harness 5040 or 5140 to provide both improved force vector orientation and controlled contact location to minimize wedging. Similarly, reinforcement 5250 at bend 5250 of harness 5240 may be incorporated into a harness having bumps 5050, 5150, providing both deformation resistance and precise contact control. These combinations allow for tailored solutions that address multiple aspects of sideloading simultaneously, enabling optimization for specific manufacturing constraints or performance requirements. The selection of a particular combination may be determined based on the binding plate being utilized, with certain release harness features being particularly complementary to specific binding plate configurations some of which are described below.

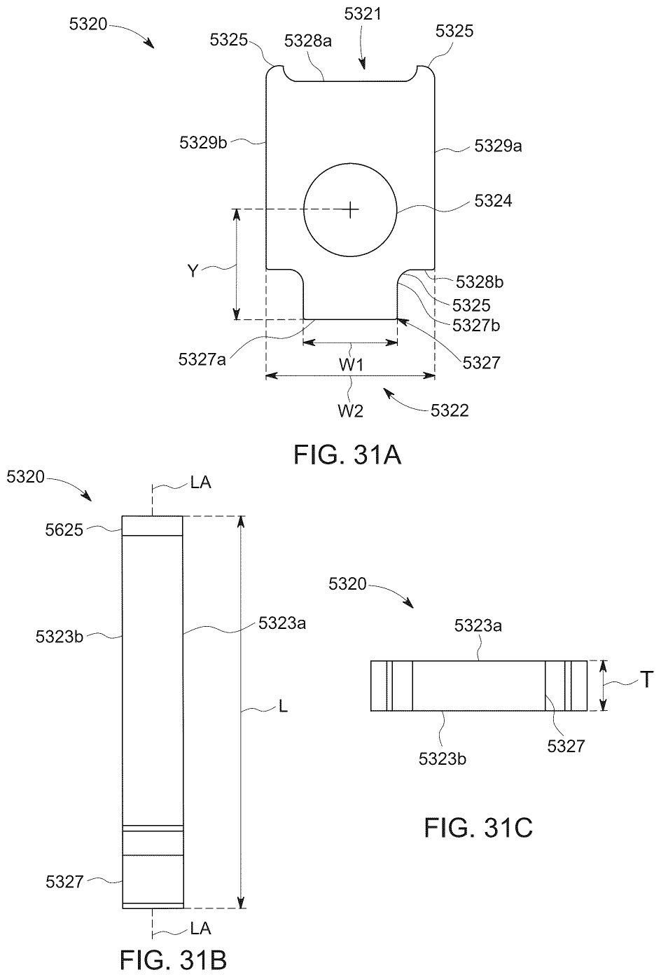

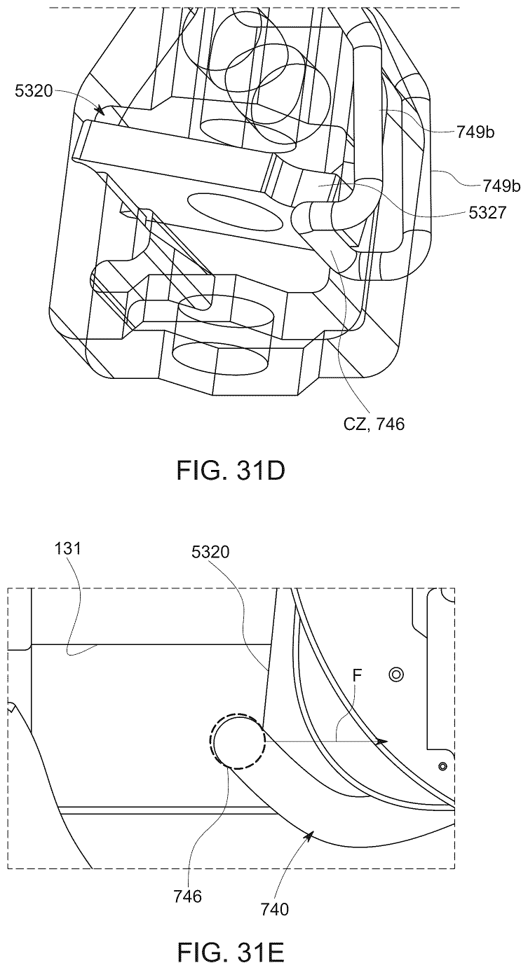

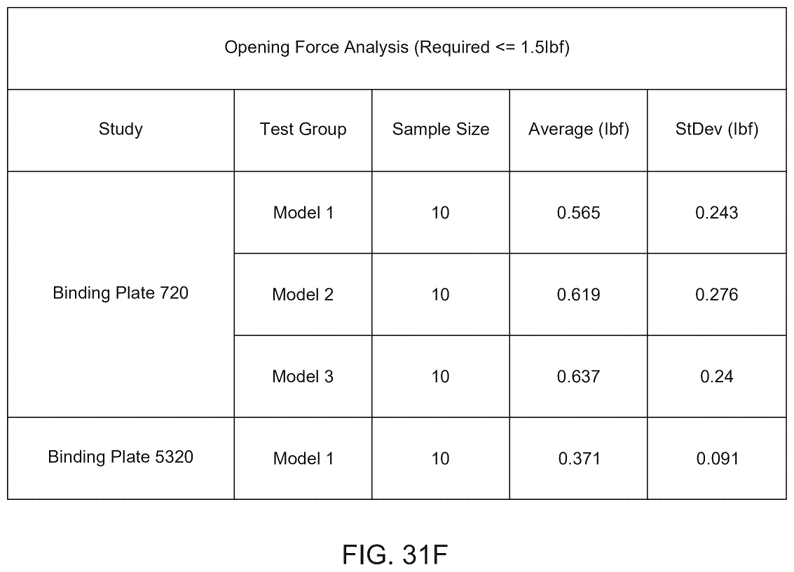

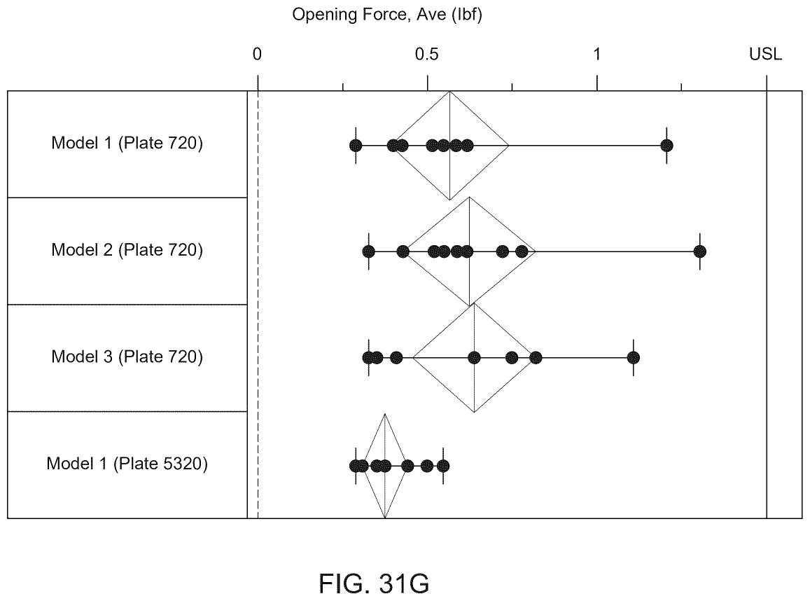

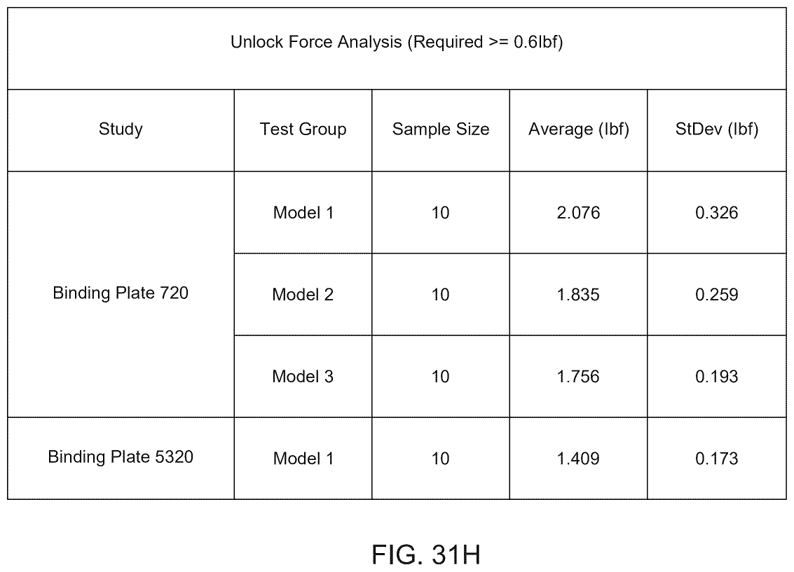

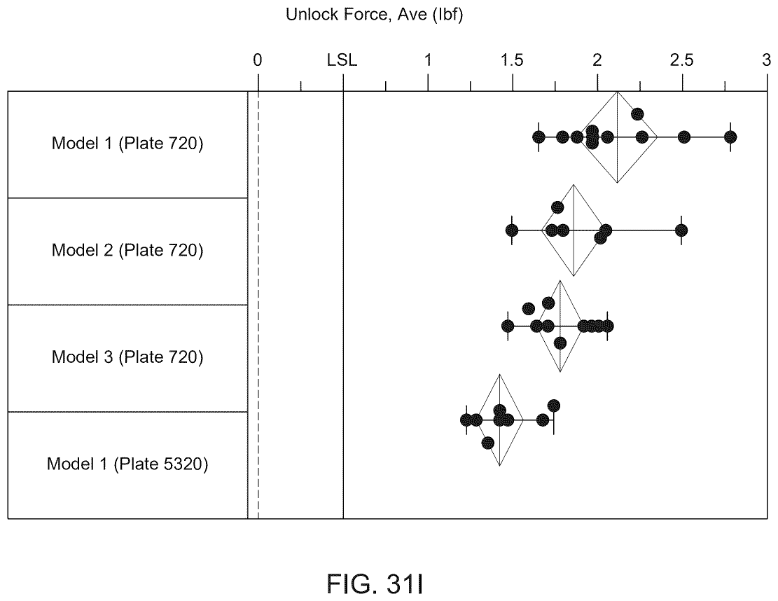

Additionally or alternatively, side loading may be addressed through binding plate modification. FIGS. 31A-31E depict a binding plate 5320 according to another embodiment of the present disclosure. Binding plate 5320 is like binding plate 720 with the exception that it does not include ears like ears 725b at the second side 5322 thereof and instead includes a tab 5327 extending from second side 5322.

In this regard, binding plate 5320 extends lengthwise between a first end 5321 and a second end 5322. First end 5321 defines a first end edge 5328a and a pair of ears (or projections) 5325 extending from first end edge 5328a at opposite sides of plate 5320 such that first end edge 5328a is recessed relative to ears 5325. Tab 5327 extends from the second end 5322 and along a longitudinal axis of binding plate 5320. As shown in FIG. 31A, tab 5327 has a width W1 and binding plate 5320 has a width W2 extending between side edges 5329a, 5329b, which is larger than W1. This forms cutouts at both sides of tab 5327 for the wire structure of release harness 740. The interface where tab 5327 joins with second end edge 5328b of plate 5320 forms a fillet 5325 merging with straight end edge 5328b of binding plate 5320 and straight side edge 5327b of tab 5327. Tab 5327 defines the second end 5322 of binding plate 5320 and has an end edge 5327a. Binding plate 5320 has a length L extending from first end 5321 to second end 5322. Binding plate 5320 also has a through opening 5324 extending through top and bottom surfaces 5323a, 5323b for receipt of stud 131.

In one example, the length L of binding plate 5320 is about 0.081 in. (±5%). The width W1 of tab 5327 is about 0.030 in. (±5%). The width W2 of binding plate 5320 is about 0.054 in. (±5%). The thickness T of the plate is about 0.0125 in. (±5%). The distance Y from the center of through hole 5324 to end edge 5327a of tab 5327 is about 0.036 in. (±5%).

The dimensions of tab 5327 and the corresponding cutout structure are specifically configured to prevent harness wedging. The tab length and cutouts are configured such that when fixation device 112 is locked, the gap created between edge 5328b of binding plate 5320 and adjacent components (e.g., lock housing 710) is always less than the diameter of harness wire (e.g., 0.008 in.), thereby preventing harness 740 from wedging in the gap formed therebetween. More preferably, this gap is less than three-quarters of the wire diameter at the top of the tolerance range for a maximum gap. In optimal configurations, the average gap measured across multiple devices is approximately half the harness wire diameter. This dimensional relationship ensures reliable operation by preventing harness wedging during locking operations.

In specific embodiments, the ratio of the gap dimension to the harness wire diameter is maintained below 1.0, preferably below 0.75, and most preferably at approximately 0.5. For a harness wire diameter of 0.008 in., the gap would be maintained below 0.008 in., preferably below 0.006 in., and most preferably at approximately 0.004 in. These dimensional relationships may be achieved through various combinations of tab length, pocket depth, and overall binding plate dimensions.

FIG. 31D illustrates release harness 740 in contact with second end 5322 of binding plate 5320. Harness 740 wraps around tab 5327 such that the contact zone CZ is centered on foot 746 of release harness 740 through direct contact with tab 5327. The narrow width W1 of tab 5327 facilitates favorable contact with foot 746 and also provides clearance for the wire structure of harness 740, particularly straight segments 749b and the bend features interconnecting foot 746 with straight segments 749b, so as to prevent harness 740 from becoming trapped or wedged as can occur with the ears 725b of plate 720.

FIG. 31E illustrates the force vector F which is generated between plate 5320 and harness 740 during unlocking upon tensioning of lock line 102. As shown, the force vector F is primarily in the axial direction with minimal to no side force component. Thus, tab 5327 creates a more defined contact point for harness foot 746, resulting in better-controlled force transmission between components. By eliminating the ear features 725b of plate 720 where wedging can occur between binding plate 720 with SSH 740, binding plate 5320 reduces physical interference between components and limits unwanted lateral translation of binding plate 5320 during unlocking operations. Therefore, tab 5327 works synergistically with SSH 740 to create a balanced system that capitalizes on the efficiency benefits of harness 740 while mitigating its potential drawbacks. As such, binding plate 5320 helps reduce variability in operation that can occur due to side loading thereby creating more predictable lock behavior across a range of operational conditions.

The performance benefits of binding plate 5320 have been demonstrated through testing of binding plate 5320 in a fixation device, like that of fixation device 112, relative to binding plate 720 in a similar fixation device. In this regard, opening force and unlock force testing was performed. Binding plate 720 was tested in three separate fixation device 112 models where the primary difference between models was the width of the distal element 120. Binding plate 5320 was tested in a single model of fixation device 112 which most closely reflected the second model of the binding plate 720 testing. The opening force testing results are shown in the table of FIG. 31F and graph of FIG. 31G. The closing force testing results are shown in the table of FIG. 31H and the graph of FIG. 31I.

As illustrated, the results of the binding plate 5320 testing revealed unexpectedly significant performance improvements compared to that of binding plate 720. Regarding opening force testing, FIGS. 31F and 31G demonstrate a surprisingly substantial reduction in opening force (0.371 lbf for binding plate 5320 as compared to 0.565-0.637 lbf for binding plate 720) along with unexpectedly improved consistency as evidenced by lower standard deviation (0.091 lbf for binding plate 5320 versus 0.240-0.276 lbf for binding plate 720). All testing values remained well below the maximum required opening force threshold of 1.5 lbf, with the magnitude of improvement exceeding initial design expectations. This result was particularly surprising because conventional design wisdom suggested that the narrow tab 5327 of binding plate 5320 would likely cause edge wedging that would result in side loading issues similar to or worse than those observed with binding plate 720. Contrary to these expectations, the narrow tab configuration of binding plate 5320 demonstrated consistently superior performance. Another unexpected benefit observed during testing was that, unlike conventional binding plate configurations (e.g., binding plate 620) where performance can degrade under increased load, the opening force of binding plate 5320 remains remarkably stable even as applied loads increase, providing more predictable operation across a range of physiological conditions.

Regarding unlock force testing, FIGS. 31H and 31I surprisingly and unexpectedly demonstrated that, while the unlock force for binding plate 5320 (1.409 lbf) was lower than that of binding plate 720 (1.756-2.076 lbf), it remained well above the required minimum threshold of 0.6 lbf while simultaneously showing surprisingly improved consistency as evidenced by the lower standard deviation (0.173 lbf versus 0.193-0.326 lbf). Perhaps most surprising was the improvement in performance ratio between unlock force and opening force. Binding plate 720 had unlock-to-opening force ratios of 2.76-3.68, while the modified binding plate 5320 achieves an unexpectedly higher ratio of 3.80, providing enhanced balance between ease of opening and security against unintended unlocking. Thus, these testing results reveal the geometry of binding plate 5320 mitigates or eliminates the potential impact of side loading during locking and unlocking cycles.

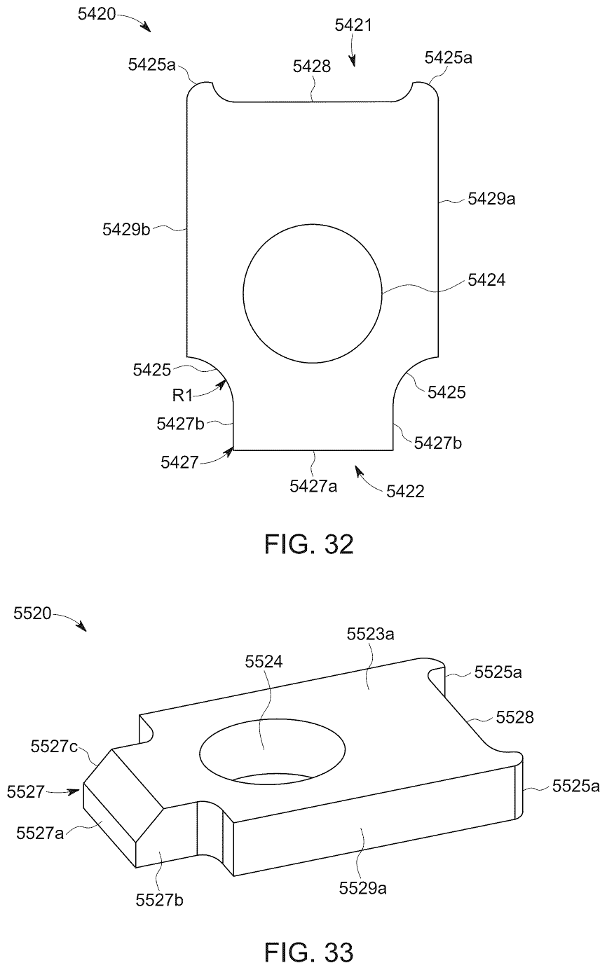

Referring now in addition to FIG. 32, which depicts a binding plate 5420 according to another embodiment of the present disclosure. Binding plate 5420 is similar to binding plate 5320, with the exception that binding plate 5420 is configured with a smoothed tab 5427 characterized by fillets 5425 that each have a radius R1 and each extend from a side edge 5427b of tab 5427 to a corresponding side edge 5429a, 5429b of binding plate 5420. Each radius R1 forms a concave curve that is complementary to the rounded wire structure of the release harness engaged to binding plate 5420. For example, when harness 740 is engaged to binding plate 5420, first and second straight segments 749b may engage and nest within the curvature of a corresponding fillet 5425. The complementary radius R1 of each fillet 5425 may reduce wear on both the binding plate 5420 and harness 740 over repeated use cycles.

Referring now in addition to FIG. 33, which depicts a binding plate 5520 according to another embodiment of the present disclosure. Binding plate 5520 is similar to binding plate 5320, with the exception that binding plate 5520 is configured with a chamfered tab 5527 that has an angled cut forming a chamfered surface 5527c thereby creating a tapered profile in a side view. The chamfered surface 5527c on tab 5527 intersects a top surface 5523a of binding plate 5520 and intersects with an end edge 5527a of tab 5527. For example, the angle cut may be at a 45-degree angle. Thus, tab 5527 has a longer length proximate a bottom surface of binding plate 5520 than proximate the top surface 5523a of binding plate 5520. This configuration may help overcome tolerance challenges by reducing the potential of tab binding-up against a wall of a lock housing, such as lock housing 710, while also closing a gap between the lock housing and binding plate 5520 to avoid wedging of a release harness, such as harness 740. In other words, the longer tab length proximate the bottom side of tab 5527 closes the gap with lock housing 710 to prevent the wire structure of harness 740 from wedging, while the minimized dimension proximate the top surface 5523a minimizes risk of tab 5527 contacting or binding-up against an inner surface of lock housing 710.

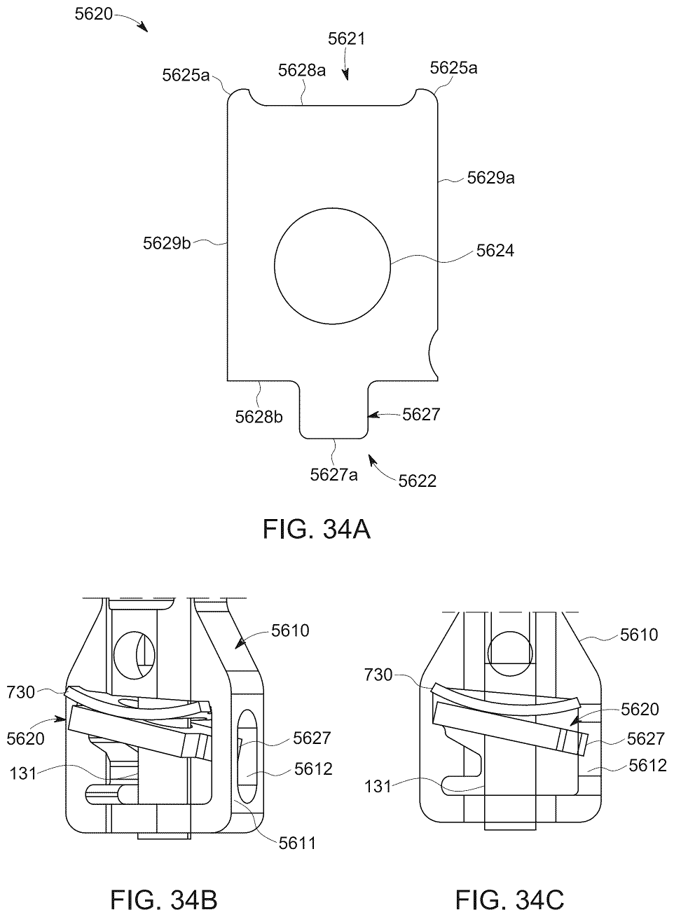

Referring now in addition to FIGS. 34A-34C, which depict a binding plate 5620 and a lock housing 5610 according to further embodiments of the present disclosure. Binding plate 5620 is similar to binding plate 5320, with the exception that binding plate 5620 is configured with a finger 5627 extending from second end edge 5628b of plate 5620 and terminating at end edge 5627a of finger 5627. Finger 5627 is generally longer and narrower than tab 5527a. The lock housing 5610 has walls 5611 that house the lock components including binding plate 5620, and one of such walls 5611 opposing the finger 5627 has an elongate slot 5612 configured to receive the finger 5627, thereby allowing the finger 5627 to be received and rotated through slot 5612 during locking and unlocking. Lock housing 5610 is coupled to a coupler, such as coupler 460, which is configured to couple to a catheter shaft, such as catheter shaft 411. Finger 5627 also extends between sides 741a, 741b of release harness 740 so that foot 746 engages finger 5627 from below and serves to rotate plate 5620 between the locked and unlocked positions. As plate 5620 is rotated, finger 5627 moves within slot 5612. This configuration prevents the wire structure of harness 740 from wedging through guided movement of binding plate 5620. Additionally, centered contact between foot 746 of release harness 740 minimizes side loading force components. Thus, the tab-and-slot arrangement provides controlled movement during locking and unlocking operations, preventing lateral displacement by physically constraining the movement path of binding plate 5620.

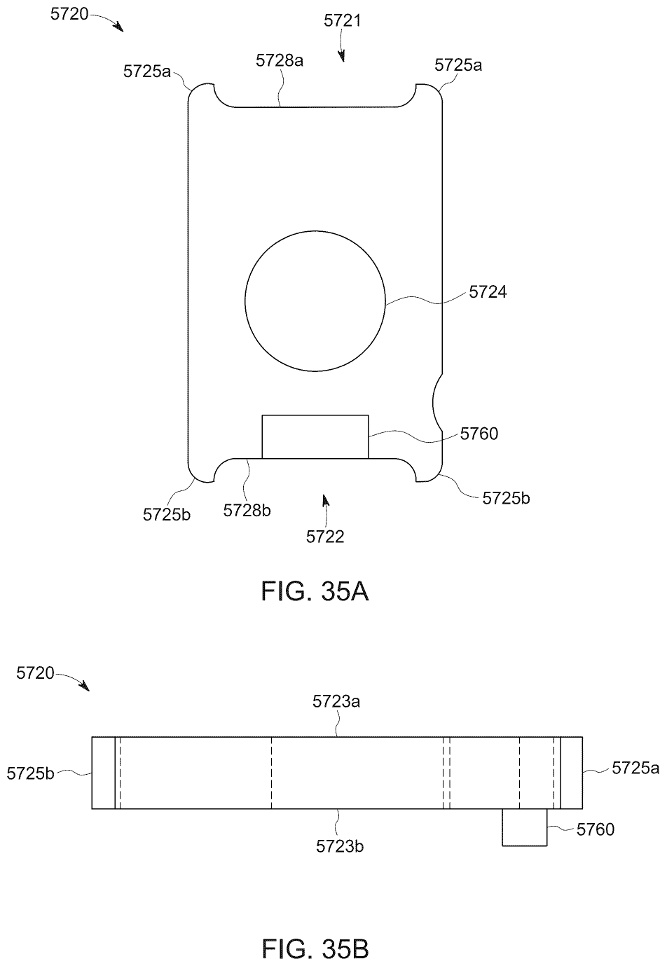

Referring now in addition to FIGS. 35A-35B, which depict a binding plate 5720 according to another embodiment of the present disclosure. Binding plate 5720 is similar to binding plate 720, with the exception that binding plate 5720 is configured with a bump 5760 that extends from a bottom surface 5723b and is located at the second end 5722 of binding plate 5720 between ears 5725b at this end. Bump 5760 is configured to be disposed between sides 741a, 741b of release harness 740 and offset from foot 746 when harness 740 is engaged therewith so that harness 740 wraps around bump 5760. In this regard, binding plate 5720 incorporates a bump or boss feature 5760 on binding plate 5720 that fixes the position of foot 746 of harness 740, thereby preventing wedging of harness 740. Since foot 746 “hooks” around this bump 5760, the moment arm for applying force onto binding plate 5720 is shorter, which results in favorably higher unlock forces, potentially improving security against unintended unlocking.

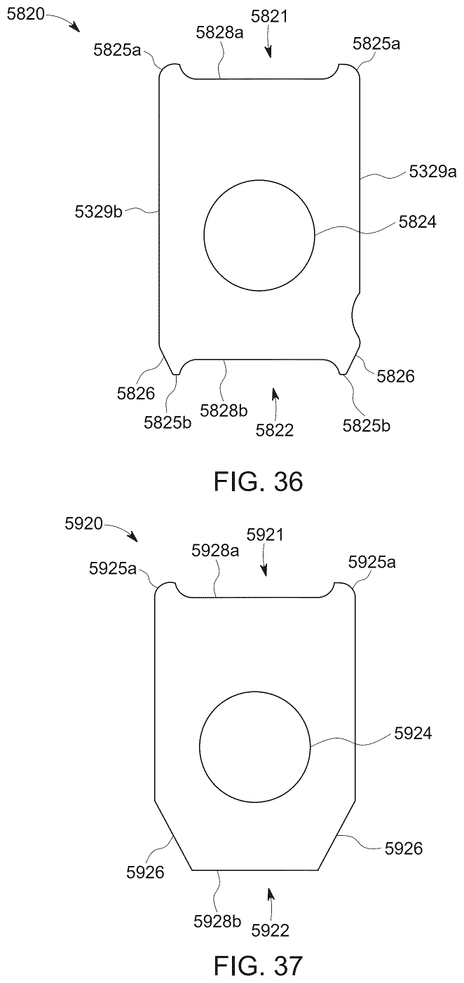

Referring now in addition to FIG. 36, which depicts a binding plate 5820 according to another embodiment of the present disclosure. Binding plate 5820 is similar to binding plate 720, with the exception that binding plate 5820 is configured with ears 5825b at the second side 5822 that have tapered side surfaces 5826 which are tapered inwardly. For example, tapered side surfaces 5826 may have a taper angle of 25 degrees relative to a corresponding side surface 5329a, 5329b of binding plate 5820. Thus, while this example maintains ears 5825b at second end 5822 of plate 5820, the tapered profiles of ears 5825b help guide contact with a release harness, such as harness 740, thereby controlling contact with the wire structure of harness 740 while maintaining structural support. The tapered ear configuration of binding plate 5820 may help to keep both components centered under loading and reduce wear on both binding plate 5820 and harness 740 over repeated use cycles by providing a guided engagement path.

Referring now in addition to FIG. 37, which depicts a binding plate 5920 according to another embodiment of the present disclosure. Binding plate 5920 is similar to binding plate 720, with the exception that binding plate 5920 does not have a second pair of ears at the second end edge 5928b. Instead, angled side surfaces 5926 intersect side surfaces 5929a, 5929b and taper inwardly toward a longitudinal axis of binding plate 5920 and intersect second end edge 5928b. Thus, binding plate has a width at second end 5922 less than that at first end 5921. In one example, the taper angle of each angled side surface 5926 taper may be about 26 degrees relative to a respective side surface 5929a, 5929b. This configuration maintains structural support while guiding foot 746 of harness 740, for example, to a more centralized contact position. In this regard, tapered edges 5926 may help improve alignment during engagement and reduce wear over repeated use cycles.

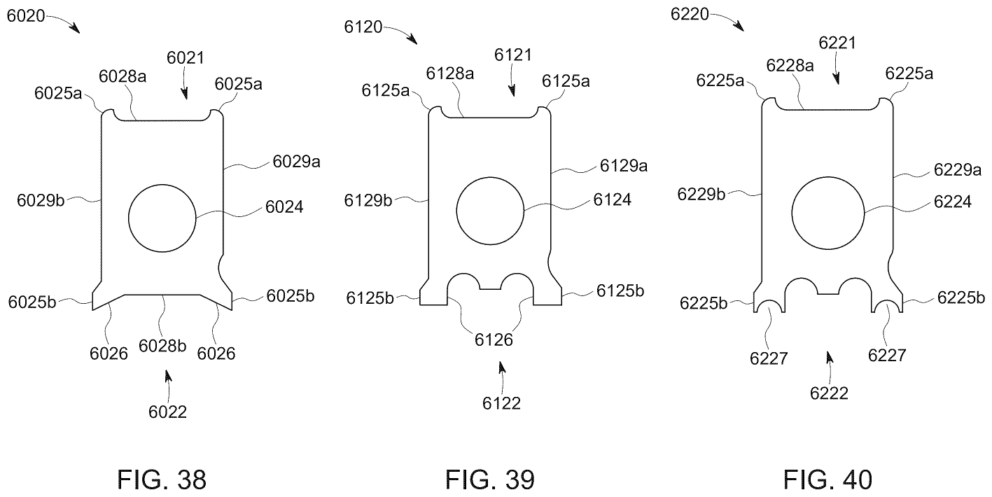

Referring now in addition to FIG. 38, which depicts a binding plate 6020 according to another embodiment of the present disclosure. Binding plate 6020 is similar to binding plate 720, with the exception that binding plate 6020 is configured with ears 6025b at the second end 6022 that are wide ears (or angled wide ears) which extend outwardly away from a longitudinal axis of binding plate 6020. As such, ears 6025b at least partially define a width of binding plate 6021 at second end 6022 that is wider than a width defined between side edges 6029a, 6029b and at first end 6021 of binding plate 6020. Additionally, ears 6025b each have an angled inner surface 6026 that is angled at an obtuse angle relative to a second end edge 6028b of binding plate 6020. Such angled inner surfaces 6026 may provide enhanced support for the wire structure of a release harness, such as harness 740, and may induce controlled inboard wedging to reduce unlocking inefficiency but with the benefit of more consistent behavior. In other words, angled inner surfaces 6026 help guide harness foot 746 to maintain alignment or centeredness with respect to binding plate 6020 as tension is applied to harness 740 via lock line 102. Such angled wide ear configuration of binding plate 6020 may enhance stability during operation while reducing wear over repeated use cycles.

Referring now in addition to FIG. 39, which depicts a binding plate 6120 according to another embodiment of the present disclosure. Binding plate 6120 is similar to binding plate 720, with the exception that binding plate 6120 is configured with ears 6125b at the second end 6122 that are wide and extended ears (or extended wide ears) which extend outwardly away from a longitudinal axis of binding plate 6120. As such, ears 6125b at least partially define a width of binding plate 6120 at second end 6122 that is wider than a width defined between side edges 6129a, 6129b and at first end 6121 of binding plate 6120. Additionally, ears 6125b have an inner surfaces 6126 that are opposed to each other and parallel to each other. This configuration helps create flat contact with the wire structure of a release harness, such as release harness 740, and helps control the harness equally on both sides thereof. In other words, the extended wide ears 6125b have flat surfaces 6126 opposing each other and being parallel to each other, unlike the angled wide ears 6025b which have angled inner surfaces 6026. This parallel ear configuration provides consistent engagement surfaces that may reduce asymmetrical wear and improve operational reliability.

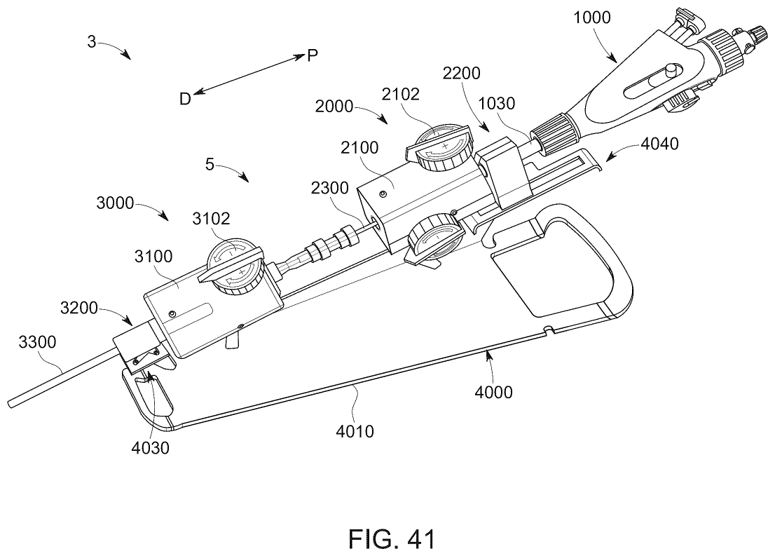

Referring now in addition to FIG. 40, which depicts a binding plate 6220 according to another embodiment of the present disclosure. Binding plate 6220 is similar to binding plate 6120, with the exception that its wide and extended ears 6225b each have a circular cutout configured to receive the wire structure of a release harness, such as release harness 740. In this regard, when harness 740 is engaged with binding plate 6220, the wire structure, such as straight segments 749b of harness 740, nest within cutouts 6227 thereby providing controlled positioning of harness 740 relative to binding plate 6220. Such cutout configuration of ears 6225b creates a complementary engagement with harness 740 which may reduce wear on both the binding plate 6220 and harness 740 over repeated use cycles by providing a precise nesting location.

It should be understood that any of the modified binding plates described herein (5320, 5420, 5520, 5620, 5720, 5820, 5920, 6020, 6120, and 6220) can be used in conjunction with any of the aforementioned single-sided harnesses (740, 5040, 5140, and 5240) to achieve the desired reduction in side loading while maintaining reliable locking and unlocking performance. For example, binding plate 5320 with its tab 5327 can be paired with reinforced harness 5140 to provide both optimized force transmission and resistance to harness deformation under load. In another example, binding plate 5720 with its bump 5760 can work effectively with harness 5040 having the single or double bump configuration on foot 5046, creating complementary contact geometry that further enhances performance consistency. In a further example, binding plate 6020 with angled wide ears 6025b can function with single-sided harness 740 by providing controlled inboard wedging that compensates for the non-reinforced foot 746. In yet a further example, binding plate 5620 with finger 5627 moveable within slot 5610 can be combined with any of the harness configurations mentioned above, as the guided movement of binding plate 5620 reduces side loading regardless of the specific harness configuration employed. These combinations provide flexibility in addressing various manufacturing constraints, tolerance issues, and performance requirements while maintaining the core benefit of reliable, consistent operation. In addition, bias element 730 may be configured in any orientation to facilitate locking in any of the aforementioned locking assembly configurations. For example, bias element 730 may be oriented such that its convex curvature contacts any one of the aforementioned binding plates at its central portion. Alternatively, bias element 730 may be flipped in orientation such that its concave side faces the respective binding plate and legs 734 of biasing element 730 may instead engage the binding plate.

It is to be understood that the fixation devices and components thereof described above are provided as examples are not to be considered as limiting to fixation devices suitable for use with other aspects of the disclosure.

FIG. 41 depicts various components of an exemplary interventional system 3 which may be utilized to implant a fixation device (e.g., fixation device 112) within a target valve. Such components may include delivery system 1000, steerable guide system 5, and stabilizer 4000. As described in more detail below, delivery system 1000 may generally include a delivery catheter handle 1010 and delivery catheter 1020. Delivery catheter handle 1010 may include a plurality of controls for steering fixation device 112 to a target valve and for controlling various features of fixation device 112, such as distal and proximal elements 120, 140, and for releasing fixation device 112 from delivery catheter 1020, for example. Additionally, steerable guide system 5 may generally include a plurality of guide catheter assemblies 2000, 3000 which may be configured to provide a conduit to help guide delivery catheter 1020 and fixation device 112 to a target valve, such as a mitral valve or a tricuspid valve, for example, and for optimal positioning of the same above a valve plane of the target valve. Stabilizer 4000 may support delivery system 1000 and steerable guide system 5 and may facilitate stable and controlled movement of fixation device 112 in-situ.

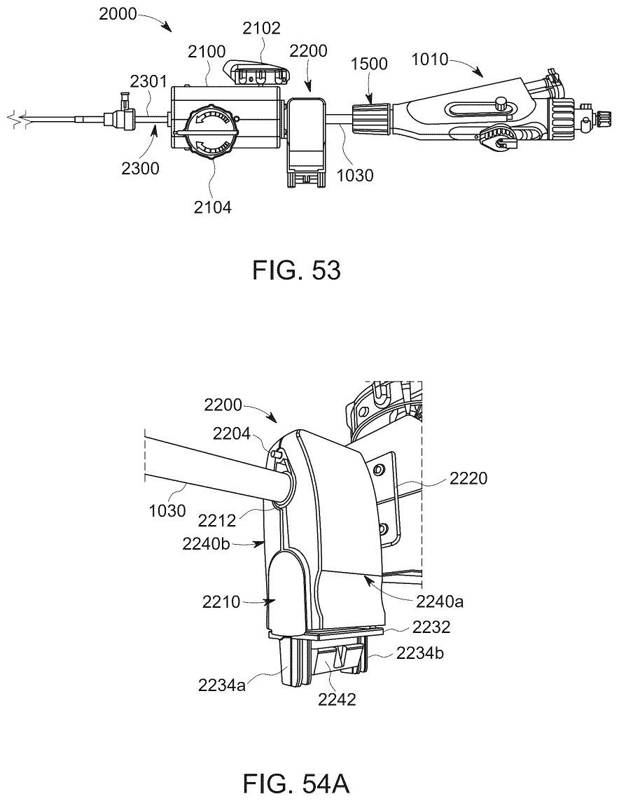

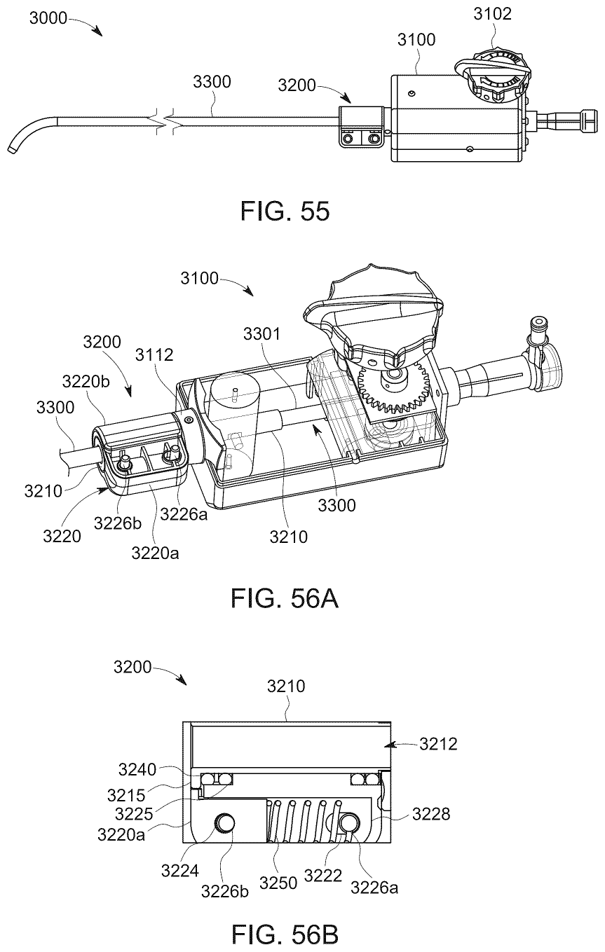

FIGS. 42A-56B depict delivery system 1000 according to an embodiment of the present disclosure. Delivery system 1000 may include a delivery catheter handle 1010 and a delivery catheter 1020 which may extend distally from delivery catheter handle 1010. The components of delivery system 1000 are configured to cooperatively function as an integrated unit for controlled and precise delivery of fixation device 112 to a target valve. Handle 1010 incorporates a plurality of control assemblies that provide the operator with tactile feedback and precise control over the positioning, deployment, and release of fixation device 112. Delivery system 1000 may also include an actuator rod 170, one or more proximal element lines 101, such as proximal element lines 101a and 101b, and one or more lock lines 102 which may extend from respective controls in handle 1010 and through delivery catheter 1020 to a fixation device 112 coupled to delivery catheter 1020, as shown in FIG. 42B and as described in more detail below.

According to one example of delivery catheter handle, delivery catheter handle 1010 may include a housing or main body 1012, a positioner assembly 1100, a gripper control assembly 1200, a fluid management assembly 1300, a lock control assembly 1400, and a delivery catheter fastening assembly 1500. Actuator rod 170 may extend from positioner assembly 1100, and positioner assembly 1100 may be configured to actuate actuator rod 170 in a proximal-distal direction for moving distal elements 120 between open, closed, and inverted positions, examples of which are described above. Positioner assembly 1100 may also be configured to release actuator rod 170 from fixation device 112 for deployment of fixation device 112. Delivery device handle 1010 may also include a deployment control system 1170 that may be coupled to positioner assembly 1100 for prevention of inadvertent deployment of fixation device 112 until desired. Proximal element lines 101a, 101b may extend from gripper control assembly 1200, and gripper control assembly 1200 may be configured to actuate proximal elements lines 101a, 101b to move proximal elements 140 between their raised and lowered positions, as described above. Lock line 102 may extend from lock control assembly 1400, and lock control assembly 1400 may be configured to actuate lock line 102 and correspondingly lock 300 of fixation device 112 between the locked and unlocked configuration, as described above. Additionally, lock control assembly 1400 may be configured to release lock line 102 from fixation device 112. Actuator rod 170, proximal element lines 101a, 101b, and lock line 102 may be directed into the fluid management assembly 1300 which itself may be coupled to delivery catheter 1020 for providing sterile fluid through one or more lumens of delivery catheter 1020. During a transcatheter procedure, it may be desirable to advance or retract delivery catheter 1020 relative to guide catheter system 5 to help position fixation device 112 within a target valve. Delivery catheter fastening assembly 1500 may be configured to selectively secure and unsecure delivery catheter 1020 so that it can be freely advanced or retracted relative to steerable guide system 5 and secured in a desired position so that delivery catheter 1020 is constrained from axially translating relative to steerable guide system 5. Each of these features are described in more detail below.

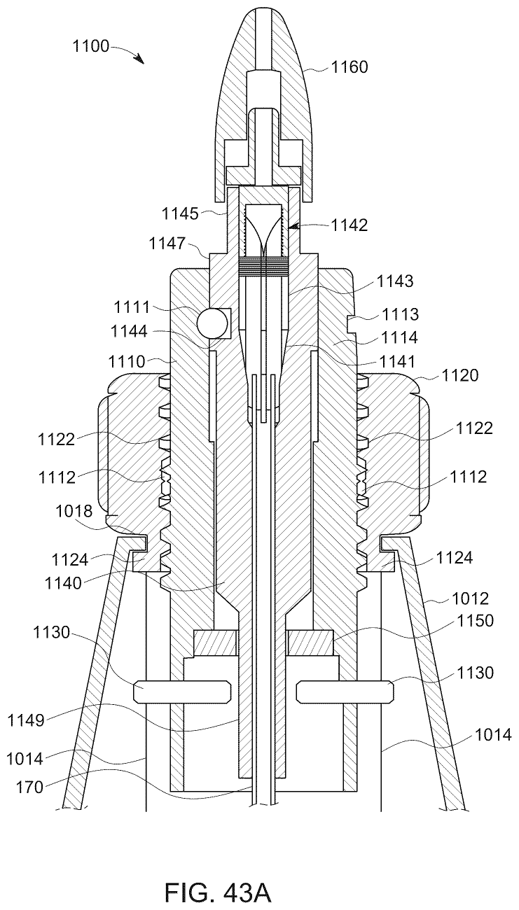

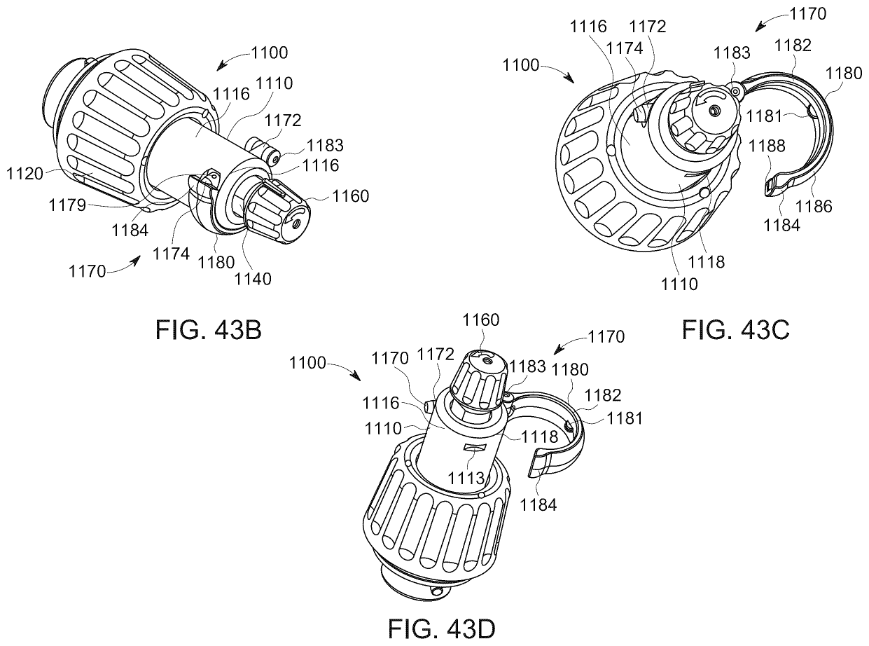

FIGS. 42B and 43A-43D depict positioner assembly 1100 according to an embodiment of the present disclosure. In one example, positioner assembly 1100 may generally include a slider 1110, an actuator knob 1120, one or more guide pins 1130, an actuator shaft 1140, a bearing 1150, and an actuator rod handle 1160.

According to various examples, slider 1110 may be substantially cylindrical and may have an axial channel 1114 that extends entirely along its length from a proximal end to a distal end thereof. A transverse opening 1111 may extend through slider 1110 near its proximal end such that it extends through opposing sides of slider 1110 and intersects channel 1114. In one example, transverse opening 1111 is offset relative to a longitudinal axis of slider 1110. Nonetheless, in such embodiment, transverse opening 1111 may be oriented generally perpendicular (±5 degrees) relative to the longitudinal axis of slider 1110. Also at the proximal end of slider 1110, a notch 1113 may extend radially inwardly into slider 1110. Such transverse opening 1111 may be configured to releasably receive a deployment pin of a deployment system, and notch 1113 may be configured to receive a protuberance of such deployment system, as described further below. At a distal end of slider 1110 in one example, a pair of guide pins 1130 may extend radially outwardly therefrom and may correspondingly engage grooves 1014 within delivery handle housing 1012. This pin 1130 and groove 1014 engagement may rotationally constrain slider 1110 relative to housing 1012 while permitting translation along housing 1012. Such guide pins 1130 may, in some examples, be orthogonal to the longitudinal axis of slider 1110 and may be press-fit to slider 1110, threaded to slider 1110, or integral with slider 1110, for example. Slider 1110 may also include external threads 1122 that extend helically along an outer surfaces thereof and that extend along at least a portion of its length, as best shown in FIG. 43A. In this regard, threads 1112 may extend along a length of slider 1110 disposed between transverse opening 1111 and guide pins 1130.

Actuator knob or actuator control 1120 may be annular such that it has a generally cylindrical exterior and interior. However, actuator knob 1120 can have other shapes, such as a conical shape, for example. As shown in FIG. 43A, in one example, actuator knob 1120 may extend circumferentially about slider 1110 and may have internal threads 1122 that engage external threads 1112 of slider 1110 such that rotating actuator knob 1120 in a first direction translates slider 1110 in a proximal direction, and rotating actuator knob 1120 in a second direction translates slider 1110 in a distal direction. Actuator knob 1120 may be rotatably connected to a proximal end of delivery handle housing 1012 while being translationally constrained. For example, actuator knob 1120 may include a circumferential lip 1124 that defines a circumferential groove that receives a corresponding lip 1018 of housing 1012, as shown in FIG. 43A. Thus, with actuator knob 1120 translationally constrained, and slider 1110 rotationally constrained via pins 1130 and grooves 1014, slider 1110 is free to translate relative to actuator knob 1120 upon its rotation.

According to one example, actuator shaft 1140 may be positioned within axial channel 1114 of slider 1110, as shown in FIG. 43A, and may be rotatable and slidable therein. Actuator shaft 1140 may be connected directly or indirectly to a proximal end of actuator rod 170. In this regard, actuator shaft 1140 may include a connection feature for connecting to actuator rod 170 in a secure manner such that it prevents relative rotation between actuator shaft 1140 and actuator rod 170. For example, actuator shaft 1140 may have a central opening that receives the proximal end of actuator rod 170 and a collet 1142 disposed within the central opening. Such collet 1142 may be configured to grasp or otherwise engage and secure actuator rod 170 to actuator shaft 1140. For example, collet 1142 may be threaded to actuator shaft 1140 and have fingers 1143 that are cammed inwardly by sloping or tapered inner surfaces 1141 of actuator shaft 1140 as collet 1142 is advanced distally within the central opening. Alternative connection features are also contemplated, such as a crimp ring and direct threaded engagement between actuator shaft and actuator rod, for example.

Actuator shaft 1140 may also have a proximal portion 1145, a distal portion 1149, and an intermediate portion 1147. In some examples, an actuator rod handle 1160 may be connected to proximal portion 1145 to help facilitate manual manipulation of actuator rod 170 when desired to release fixation device 112. In this regard, proximal portion 1145 may be positioned at least partially outside of axial channel 1114 of slider 1110. On the other hand, intermediate portion 1147 may be at least partially disposed within channel 1114. Intermediate portion 1147 may have a larger cross-sectional dimension than proximal and distal portions 1145, 1149 and may include a notch 1144 extending radially inwardly. Such notch 1144 may be configured to align with transverse opening 1111 of slider 1110, as shown in FIG. 43A, so that a deployment pin 1172 (FIGS. 43B-43D) extending through transverse opening 1111 and notch 1144 prevents actuator shaft 1140 from rotating and translating relative to slider 1110, as discussed in more detail further below.

In various examples, a bearing 1150 may be disposed within axial channel 1114 of slider 1110, and distal portion 1149 of actuator shaft 1140 may be removably received within bearing 1150, as shown in FIG. 43A. Bearing 1150 may be secured to slider 1110 via a press-fit or threaded connection, for example. Bearing 1150 may be a one-way bearing such that, when distal portion 1149 of actuator shaft 1140 is received within bearing 1150, actuator shaft 1140 is rotatable in a first direction but prevented from rotation in a second direction, for example. As mentioned above, in one example, actuator rod 170 may be released from fixation device 112 by rotating actuator rod 170 in the first direction which may unthread a distal end of actuator rod 170 from stud 131 or base 139 of fixation device 112. Thus, in some examples, bearing 1150 may allow actuator rod 170 to be rotated for release but prevent rotation in the opposite direction which may help prevent inadvertent overtightening. An exemplary positioner assembly with one-way bearing is disclosed in U.S. Pat. No. 10,660,625, the disclosure of which is hereby incorporated by reference herein in its entirety.

Actuator shaft 1140 may be coupled to slider 1110 via a deployment system 1170, for example. An exemplary deployment system 1170 is depicted in FIGS. 43A-43D and may include one or more of transverse opening 1111 of slider 1110, notch 1144 of actuator shaft 1140, and a deployment pin 1172 which may be releasable received within transverse opening 1111 and notch 1144. Deployment system 1170 may also include a handle 1170. Handle 1180 may connect to the deployment pin 1172 via a hinge 1183 in one example. In one example, handle 1180 may be pivotable relative to slider 1110 when pin 1172 is disposed therein so as to pivot between a locked configuration (shown in FIG. 43B) and an unlocked configuration (shown in FIGS. 43C and 43D). In the depicted embodiment, handle 1180 may extend around a portion of slider 1110 (e.g., about substantially one half of the slider 1110 or more). Handle 1180 may, for example, comprise a curved profile 1182 that corresponds to a curved profile 1116 of slider 1110, which advantageously minimizes a profile of the deployment system 1170 in the engaged configuration. This permits removal of pin 1172 when handle 1180 is rotated to the unlocked configuration and prevents removal of pin 1172 when handle 1180 is in the locked configuration. Handle 1180 may, for example, include at least one protuberance 1181 extending from an interior surface 1182 of handle 1180 which may cooperate with indentation or notch 1113 in slider 1110. Protuberance 1181 and notch 1113 can prevent handle 1180 from inadvertently rotating off of slider 1110. Handle 1180 may also include a pin cover 1184 in one example. Pin cover 1184 may be configured to cover and retain a pin tip 1174 when handle 1180 is in the locked configuration, which can in turn retain pin 1172 within slider 1110, as shown in the example of FIG. 43B. Pin cover 1184 may have a groove 1188 which can provide a space for the pin tip 1174. When provided, such covering 1184 helps prevent inadvertent contact with pin 1172 that could result in premature disengagement. A spring biased ball 1179 may also be included in pin 1172 to help further secure pin 1172 from being inadvertently removed from slider 1110 in some examples.

In operation, positioner assembly 1100 may be utilized to move distal elements 120 between the open, closed, and inverted positions. In one example, rotating actuator knob 1120 in the first direction may translate slider 1110 and consequently actuator rod 170 proximally which may move distal elements 120 from the closed to open position and from the open position to the inverted position. On the other hand, rotating actuator knob 1120 in the second direction may advance slider 1110 and consequently actuator rod 170 distally which may move distal elements 120 from the inverted position to the open position and from the open position to the closed position. When it has been determined that the valve leaflets have been sufficiently grasped by fixation device 112, fixation device 112 may be released from delivery catheter 1020. In one example, this may be achieved by releasing handle 1180 from slider 1110 and removing pin 1172 from slider 1110 which frees actuator shaft 1140 from slider 1110. Once pin 1172 has been removed, actuator shaft 1140 and actuator rod 170 may be rotated via actuator handle 1160 in the first direction thereof to thereby release actuator rod 170 from stud 131 or base 139. Once released, actuator rod 170 can be pulled proximally relative to slider 1110 which retracts actuator rod 170 and positions it proximal to the coupling interface between shaft 111 and coupling member 160, as described above with respect to coupling system 115 of FIGS. 7A and 7B and also similarly described with respect to coupling system 315 of FIGS. 8A and 8B. Other exemplary deployment control systems and positioner assemblies that may be used in delivery system 1000 are disclosed in U.S. Pub. No. 2021/0353419 (“the '419 Publication”), the disclosure of which is hereby incorporated by reference herein in its entirety.

FIGS. 42B and 44A-44C depict gripper control assembly 1200 according to an embodiment of the present disclosure. Examples of the gripper control assembly 1200 may generally include one or more of a first element line handle 1202a, a second element line handle 1202b, and an interlock 1210 selectively coupling first and second element line handles 1202a, 120b to each other. In one example, first proximal element line 101a may be coupled to first gripper element line handle 1202a, and second proximal element line 102b may be coupled to second gripper element line handle 1202b. Each of these handles 1202a, 1202b may separately and independently actuate first and second proximal element lines 101a, 101b so as to separately and independently raise and lower proximal elements 140 between their raised and lowered positions, according to examples of the disclosure. However, when interlock 1210 couples first and second element line handles 1202a, 1202b together, first and second proximal element lines 101a, 101b and, consequently, proximal elements 140 may be actuated concurrently in some examples. In the embodiment depicted, first and second proximal element line handles 1202a, 1202b can be aligned in parallel which helps facilitate concurrent actuation. Although two proximal element line handles 1202a, 1202b are depicted, one proximal element line handle may be provided where a single proximal element line 101 is coupled to both proximal elements 140, such as in the interventional device 110′ arrangement depicted in FIG. 15. It should also be understood that a third proximal element line handle can also be provided where fixation device 112 may include a third proximal element 140 in some examples.

First and second proximal element line handles 1202a, 1202b may each include a connection mechanism for connecting to respective proximal element lines 101a, 101b. In the embodiment depicted, each element line handle 1202a, 1202b may connect to a proximal end portion 103a (see FIG. 44C) of its respective proximal element line 101a, 101b, while a second end portion 103b of each proximal element line 101a, 101b may be releasably coupled to shaft 111, such as described with respect to FIGS. 16 and 17A-17F. Thus, in the embodiment depicted, the connection mechanism of each proximal element line handle 1202a, 1202b may be configured to connect to first end portion 103a of a respective proximal element line 101a, 101b. For example, the connection mechanism can include a collet 1220 that may securely grasp proximal end portion 103a of proximal element lines 101a, 101b. As shown, first proximal element line handle 1202a may have a lumen 1203 with a central axis, and a screw 1224 therein. Screw 1224 can have a lumen 1223 with collet 1220 therein, collet 1220 may have fingers 1222, for example. First end 93a of first proximal element line 101a can be disposed within with a recess defined by fingers 1222 according to one example. When screw 1224 is tightened, collet fingers 1222 may tighten down on and grasp proximal element line 101a. In this regard, proximal element line 101a may be fixed in a tensioned or non-tensioned state. Proximal element line 101b may be coupled to proximal element line handle 1202b using a similar collet system (i.e., connection mechanism). Although the depicted embodiment includes a connection mechanism including a collet 1220, it should be understood that other connection mechanisms may be utilized for securing a first end 93a of a proximal element line 101a, 101b to a proximal element line handle 1202a, 1202b, such as a swage or a knot, for example.

One or more of proximal element line handles 1202a and 1202b may also include respective end caps 1206a, 1206b disposed at their proximal ends in some examples. Each of the end caps 1202a and 1206b can, for example, include finger grip portions, such as one or more top concave portions 1207. In various examples, one or more of end caps 1206a and 1206b may include a bump 1208 which may provide tactile feedback to the surgeon as to which handle 1206a, 1206b and corresponding proximal element 140 is being actuated. Other tactile features can be utilized such as protrusion, ridge, or roughened surface, for example.

Interlock 1210 may, for example, be moveable between an unlock position in which the first and second proximal element line handles 1202a and 1202b are independently actuatable and a locked position in which first and second proximal element line handles 1202a and 1202b are coupled together to be actuatable together. Interlock 1210 can be fixedly or moveably coupled to first proximal element line handle 1202a and releasably coupled to second proximal element line handle 1202b. Interlock 1210 can include, for example, a latch 1212 and recess 1213. Latch recess 1213 can receive latch 1212, such that proximal element line handles 1202a and 1202b are operably connected for concurrent actuation. In one example of the unlocked position, latch 1213 can be removed from recess 1213, such that the proximal element line handles 1202a and 1202b are not operatively connected and are independently actuatable. Latch 1213 can be configured to be moved linearly, rotationally, or in any suitable motion to lock and unlock the interlock 1210 as described. Also, as shown in FIG. 44B, latch 1213 and recess 1213 can have complementary dovetail shapes in various examples. The dovetail shapes can limit the proximal element line handles 1202a and 1202b from pulling away from one another when the latch 1212 is engaged with recess 1213. The dovetail shapes can also prevent interlock from switching between the lock and unlock positions. A locking lever 1214 can be provided to facilitate movement of latch 1212 in some examples. Actuating interlock 1210 between the locked position and the unlocked position can, for example, be performed with a single hand or a single finger. Either latch 1212 or recess 1213 can be disposed in either first proximal element line handle 1202a or second proximal element line handle 1202b. Other interlock examples that may be used in gripper control assembly 1200 are disclosed in U.S. Pub. No. 2021/0015614, the disclosure of which is hereby incorporated by reference herein in its entirety.

Proximal element line handles 1202a, 1202b may each be moveably coupled to a base 1201 which may be secured to housing 1012 of delivery system handle 1010 in one example. Base 1201 provides structural support for gripper control assembly 1200 and helps maintain proper alignment of each proximal element line handle 1202a, 1202b during actuation. In this regard, each proximal element line handle 1202a, 1202b and their respective connection mechanisms may be moveable in a proximal-distal direction between a first position and a second position relative to base 1201, according to one example. The movement between positions helps provide consistent tensioning of the proximal element lines 101a, 101b and corresponding movement of proximal elements 140 regardless of variations in operator technique. One example of a first position is shown in FIG. 42B and is a tensioned position in which proximal element lines 101a, 101b are tensioned so that proximal elements 140 are in their raised position. Pushing proximal element line handles 1202a, 1202b in a distal direction moves handles to their second position (see FIG. 44C) which releases the tension and correspondingly lowers proximal elements 140. Base 1201 and proximal element line handles 1202a, 1202b may together form a ball-detent mechanism or a snap-fit mechanism, for example, which may releasably and respectively secure proximal element line handles 1202a, 1202b in the first and second position. The detent mechanism provides tactile feedback to the operator confirming that handles 1202a, 1202b have reached their respective positions and helps prevent unintended movement during the procedure. As illustrated in FIG. 42B, base 1201 is disposed within housing 1012 such that, when proximal element line handles 1202a, 1202b are in an example of their second position, a majority of their respective lengths are disposed within base 1201 and handle housing 1012. This helps secure gripper control assembly 1200 and provide gripper control assembly 1200 a low profile.

A first sheath 1220a and second sheath 1220b may extend from a distal end of base 1201 and to a manifold 1320 of fluid management assembly 1300, as described in more detail below. In one example, first proximal element line 101a may extend through first sheath 1220a, and second proximal element line 101b may extend through second sheath 1220b. Sheaths 1220a, 1220b may help shield proximal element lines 101a, 101b from rubbing or snagging on other components within handle 1010 as they are actuated. Additionally, sheaths 1220a and 1220b help prevent fluid from escaping fluid management assembly 1300 and or contaminants from entering fluid management assembly 1300.

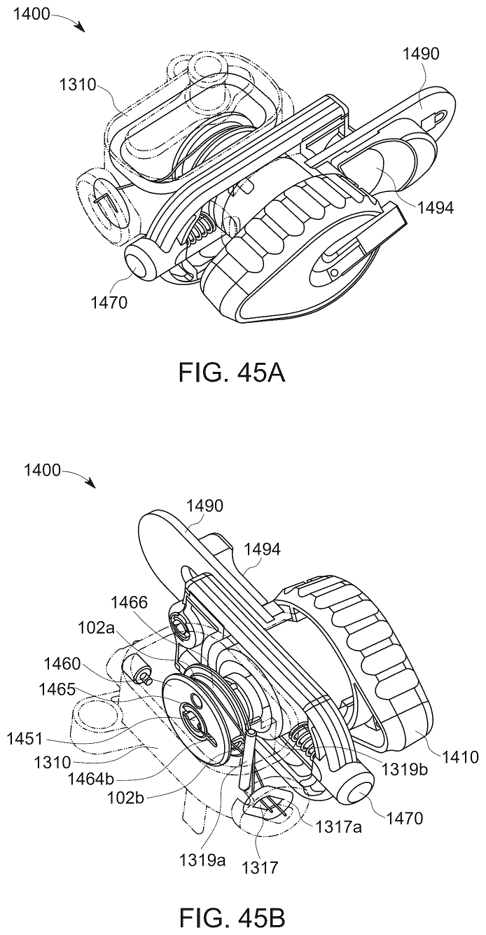

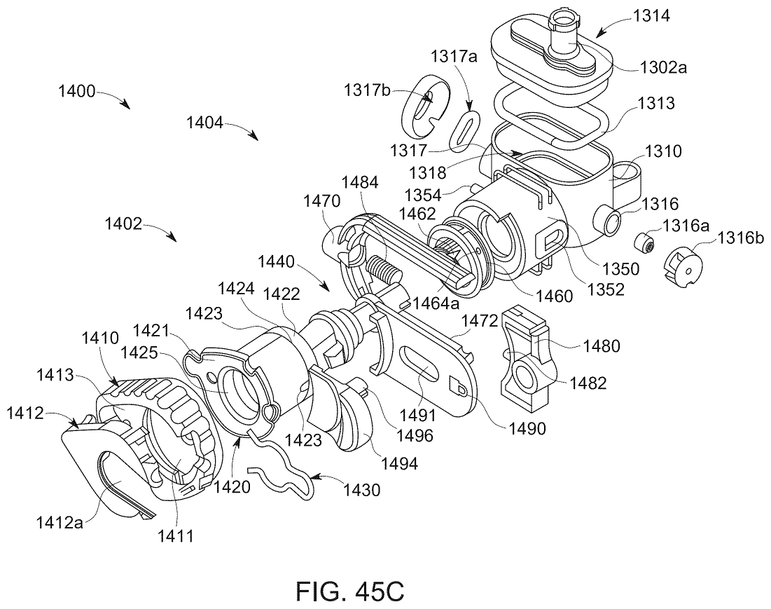

As shown in FIGS. 42B, 44A, and 45C, fluid management assembly 1300 may generally include a fluid chamber 1310, a connector 1330, and a manifold 1340 separating chamber 1310 from connector 1330 according to various examples of the disclosure. Fluid chamber 1310 may also be a lock handle housing, as described in more detail below. Thus, fluid chamber 1310 and lock handle housing 1310 are used interchangeably herein. Fluid chamber 1310 may include a chamber volume 1318 and chamber lid 1314 covering chamber volume 1318 in one example. In one example, an O-ring 1313 seals lid 1314 to fluid chamber 1310, as best shown in FIG. 45C. Chamber lid 1314 may include a first inlet/outlet port 1302a which may define a luer lock for connection to a flush bag or the like for delivering/receiving fluid to or removing air from chamber 1310 in some examples. Chamber 1310 may also include a lock handle extension (or cylindrical extension) 1350 extending from a side thereof according to various examples. Such lock handle extension 1350 may be configured to receive a lock line knob, as described further below. Additionally, fluid chamber 1310 may include a first opening 1316 at proximal end and a second opening 1317 at a distal end, as shown in the example of FIG. 45C.

Connector or fluid inlet block 1330 may be disposed distal of fluid chamber 1310 and may be configured to connect to a proximal end portion 1021 of delivery catheter 1020, such as over at least a portion of proximal end portion 1021 of delivery catheter 1020, in some examples. In this regard, proximal end portion 1021 of delivery catheter 1020 may be connected to connector 1330 such that fluid may flow between connector 1330 and delivery catheter 1020 lumens to lubricate lines 101a, 101b, 102 and shaft 170 and over an exterior of delivery catheter 1020 to ensure smooth translation of delivery catheter relative to sleeve 1030, in some examples. Connector 1330 may also include a second inlet/outlet port 1302b for fluid transfer, for example. Second port 1302b, however, may extend from an opposite side of handle 1010 from first inlet/outlet port 1302a in various examples.

Manifold 1340 may be positioned between fluid chamber 1310 and connector 1330 and may be connected to a proximal end of connector 1330, as shown in the example of FIGS. 42B and 44A. Manifold 1340 may include a shaft 1342 extending proximally therefrom which may be coupled to second opening 1317 of fluid chamber 1310 in one example. Shaft 1342 may have a plurality of lumens extending therethrough each for receipt of any one of actuator rod 170, a first end portion 102a of lock line 102, and a second end portion 102b of lock line 102 (see e.g., FIG. 49E). Second opening 1317 of fluid chamber 1310 may include an O-ring 1317a and ring retainer 1317b to provide a leak free seal between fluid chamber 1310 and shaft 1342, for example. As illustrated in the example of FIG. 44A, actuator rod 170 may extend into first opening 1316 of fluid chamber 1310 and may pass through fluid chamber 1310 into shaft 1342 of manifold 1340 where it may then be directed into delivery catheter 1020. First opening 1316 of fluid chamber 1310 may include a mandrel seal 1316a and mandrel retainer 1316b (see FIG. 45C) for sealing the interface between actuator rod 170 and fluid chamber 1310 in various examples. Proximal element line sheaths 1220a, 1220b may be connected to manifold 1340 such that proximal element lines 101a, 101b extending through sheaths 1220a, 1220b are directed through manifold 1340 and into delivery catheter 1020 according to various examples.

In one example, fluid may enter fluid management system 1300 via port 1302b and into connector 1330. Fluid from connector 1330 may then travel distally into the lumens of delivery catheter 1020 to lubricate the control elements therein, such as gripper lines 101a and 101b, and lock line end portions 102a and 102b, for example. Additionally, fluid from connector 1330 may flow distally between delivery catheter 1020 and sleeve 1330 to lubricate relative translation, as described in more detail below. Fluid may also travel proximally from connector 1330 through manifold 1340 and into sheaths 1220a, 1220b to lubricate gripper lines 101a, 101b and through lock line lumens within shaft 1342 and into fluid chamber 1310. Once fluid is located within chamber 1310, it may then travel distally through a lumen for actuator rod 170 which may pass through shaft 1342 and into delivery catheter 1020. Inlet/outlet port 1302a may be used to deair the system.

The arrangement of fluid management assembly 1300, as described above and shown in the figures, significantly reduces the volume of fluid delivered to and from delivery system handle 1310 as compared to prior delivery systems and provides a single point flush and anchoring to handle 1310 while ensuring fluid runs within delivery catheter 1020 lumens and over an outer surface of delivery catheter 1020. Such reduction may be upwards of 3.7 times that of prior delivery systems and results in a more efficient deairing of system 3 with a reduction in the amount of flush bags utilized. This efficiency is particularly beneficial during time-sensitive cardiac procedures where rapid and complete deairing is essential to prevent air embolism. Fluid volume of fluid management assembly 1300 may be about 14 cubic centimeters, for example, which represents an optimal balance between adequate fluid delivery for lubrication and minimal volume for efficient deairing. The reduced fluid volume also decreases the system's overall weight enhancing operator handling and control during delicate procedures. Fluid volume of fluid management assembly 1300 may be about 14 cubic centimeters, for example. Such reduction in volume is facilitated at least by the knob configuration of lock line assembly 1400, which is described in more detail below. Fluid management assembly 1300 also decreases overall preparation time and decreases the risk that a flush line may become tangled at least due to a reduced number of flush line connections (e.g., one total flush line connection to port 1302b). The simplified connection arrangement enables faster setup and reduces the potential for operator error. Furthermore, the arrangement of fluid management assembly 1300 reduces the number of hemostatic valves that may be needed which correspondingly increases the torque transmission ratio from delivery catheter handle 1010 to fixation device 112, resulting in more responsive and precise control during valve repair procedures.

FIGS. 30A-49G depict lock control assembly 1400 according to an embodiment of the present disclosure. Lock control assembly 1400 may actuate any one of locks 500, 600, and 700 between their respective locked and unlocked configurations, for example. Lock control assembly 1400 may, for example, also be configured to release lock line 102 from fixation device 112, 112′, and 112″. Lock control assembly 1400 may generally include one or more of a lock line handle 1402, a lock line handle housing 1310, a spool 1460, and a locking system 1404.

Referring to the example of FIG. 45C, lock line handle 1402 may generally include a lock knob 1410 and a lock handle shaft 1440 extending from lock knob 1410.

In one example, lock knob 1410 may be configured to be gripped by a user and pivoted relative delivery system handle 1010 to operate lock line 102 (as described in greater detail below). Lock knob 1410 can have a pear shape which can act as a flag or pointer to help indicate when lock knob 1410 is in a locked or unlocked position. Although shown as a pear shape, lock knob 1410 can have any suitable shape and/or configuration. Also, lock knob 1410 may have a recess 1413 which may also be pear shaped. However, other shapes are contemplated such as rectangular or oval, for example. Additionally, lock knob 1410 may have an opening 1411 (e.g., a cylindrical shaped opening) extending therethrough and intersecting recess 1413.

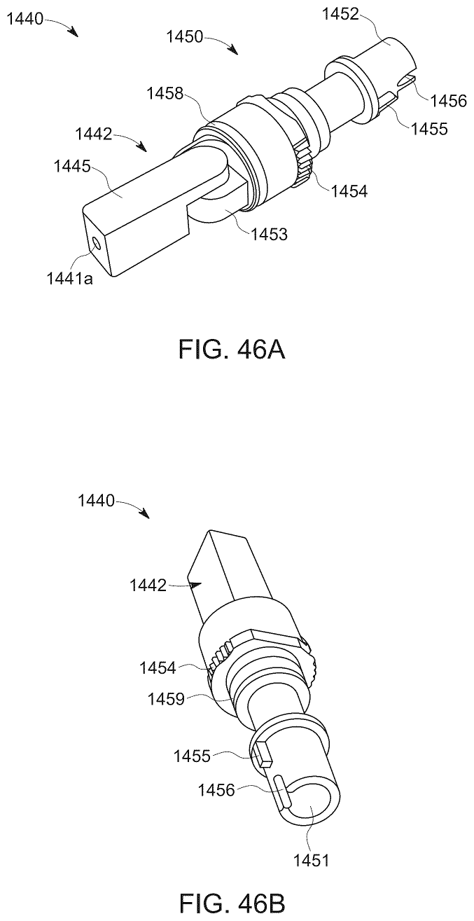

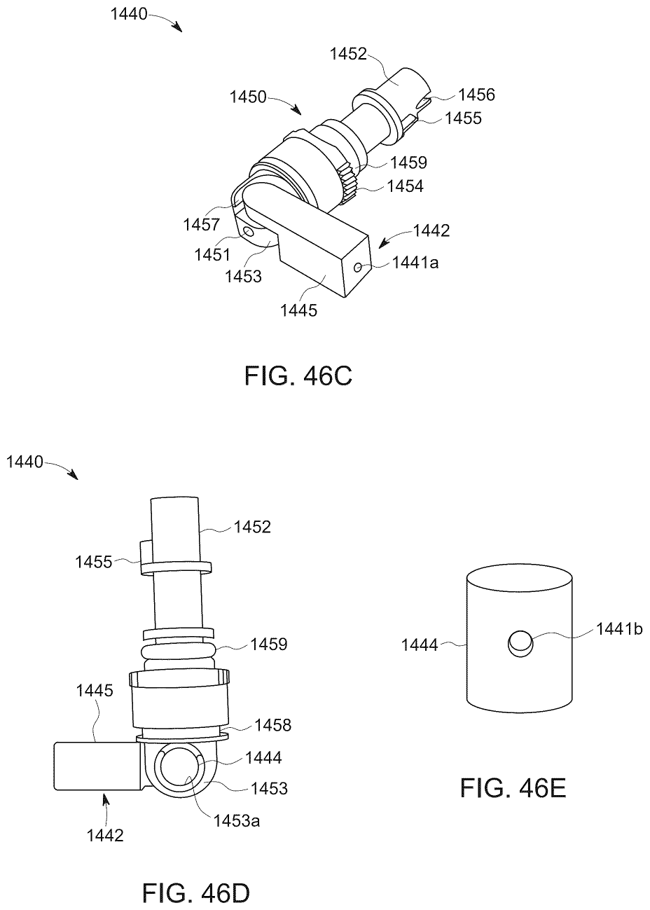

In the embodiment depicted, lock knob 1410 may include a lock knob insert 1420 and a lock knob cap 1412. Lock knob insert 1420 may include an insert body 1422 that may be cylindrical and a flange 1421 that may be connected to one end of insert body 1422. Flange 1421 may have the same (i.e., corresponding) shape as lock knob recess 1413 in various examples. Thus, where lock knob recess 1413 is pear shaped, flange 1421 may have a pear shape. Insert body 1422 may have an inner surface that may define an opening or passageway 1425 that may extend entirely through insert body 1422 in some examples. Inner surface of insert body 1422 may include a plurality of teeth or splines that may be configured to enmesh with corresponding teeth or splines 1454 on lock handle shaft 1440 (see e.g., FIG. 46C), as discussed further below. Insert body 1422 may also include a channel 1424 extending circumferentially about an exterior of insert body 1422 and may include a plurality of detents 1423 which may each extend radially inwardly and, in one example, may intersect channel 1424. In various assembled examples, flange 1421 may be disposed within recess 1413 of lock knob 1410 while insert body 1422 may extend through cylindrical opening 1411 of lock knob 1410. The shape (e.g., pear) of lock knob recess 1413 and corresponding shape (e.g., pear) of flange 1421 can limit relative rotation between lock knob insert 1420 and lock knob 1410. Lock knob cap 1412 may be connected to lock knob 1410, such as via a snap-fit connection and/or threaded connection, for example. Lock knob cap 1412 may be positioned over flange 1421 of insert 1420 which may sandwich flange 1421 between lock knob 1410 and lock knob cap 1412 thereby retaining lock insert 1420. Lock knob cap 1412 may also have an opening or a recess 1412a that may align with the opening 1425 of lock knob insert 1420, as shown in the example of FIG. 45C, and may provide clearance for a lever arm 1442 of lock handle shaft 1420 to be partially positioned therein in a locked position. Although lock knob 1410, lock knob insert 1420, and lock knob cap 1412 are shown and described as being separate components, it should be understood that each of these components can be integrated together so as to form a monolithic lock knob structure, such as by an injection molding process or additive manufacturing process, for example.

FIGS. 46A-46E depict one example of lock handle shaft 1440. Lock handle shaft 1440 may generally include one or more of a shaft body 1450 and a lever arm 1442 pivotably connected to shaft body 1450. Shaft body 1450 may include a first end portion 1452 and a second end portion 1453. An opening 1451 may extend through the length of shaft body 1450 from first end portion 1453 to second end portion 1453. As shown in the example of FIGS. 46B and 46C, opening 1451 may be larger at first end portion 1452 than at second end portion 1453. First end portion 1452 of shaft body 1450 may be substantially cylindrical and may be configured to be received within a corresponding opening 1465 in spool 1460 (see e.g., FIG. 45B). A notch 1456 may be formed in first end portion 1452, and a fin or tab 1455 may extend radially outwardly from first end portion 1452 in some examples. Fin 1455 may be configured to be received within a corresponding notch 1462 in spool 1460 (see FIG. 45C), for example. In the embodiment depicted, fin 1455 and notch 1462 may be circumferentially aligned. Lock handle shaft body 1450 may, for example, also include an O-ring 1459 positioned between first and second end portions 1452, 1453 for forming a seal between lock handle shaft 1440 and lock handle extension 1350 of lock handle housing 1310 when received therein. Additionally, shaft body 1450 may include a plurality of teeth or splines 1454 which may enmesh with the teeth of lock knob insert 1420 which may rotationally constrain lock handle shaft 1440 relative to lock knob insert 1420 when disposed therein according to various examples of the disclosure. As shown in the example of FIGS. 46C and 46D, second end portion 1453 may include a post opening or hinge opening 1453a for a corresponding post 1444 of lever arm 1442. Also, second end portion 1453 may include an abutment feature 1457 extending radially outwardly therefrom which may be configured to constrain a range of motion of lever arm 1442 when coupled to body 1450, for example.

In various examples, lever arm 1442 of lock handle shaft 1440 may include a lever body 1445 and post or hinge 1444 extending from lever body 1445. Post 1444 may be rotatably received within post opening 1453a of shaft body 1450 in some examples. Lever arm 1442 may also include a first opening 1441a that passes through lever body 1442 and a second opening 1441b that passes through post 1444 (see the example of FIG. 46E). However, in some embodiments, lever arm may only include opening 1441b passing through post. First and second openings 1441a, 1441b may be axially aligned in various examples. As shown in the example of FIG. 49F, lock line 102 may include a first end portion 102a and a second end portion 102b. First end portion 102a may be fixedly secured to shaft body 1450, such as to an inner surface thereof. Prior to removal of lock line 102 from fixation device 112, second end portion 102b of lock line 102 may extend through opening 1451 in shaft body 1450, and first and second openings 1441a, 1441b of lever arm 1445. In one example, lever arm 1445 may be rotatable about an axis of post 1444 and relative to shaft body 1442 between a first position or unlocked configuration (see FIGS. 46A, 46B, and 49F) and second position or locked configuration (see FIGS. 46C and 46D). In one example of the unlocked configuration, lever arm 1442 may be axially aligned with shaft body 1445 such that first and second openings 1441a, 1441b of lever arm 1442 are coaxially aligned with opening 1451 of shaft body 1450. This may allow second end portion 102b of lock line 102 to be passed through lock handle shaft 1440 as it is pulled away from delivery system handle 1010, as best shown in FIG. 49F. In one example of the locked configuration, lever arm 1442 may be rotated such that it is angled relative to shaft body 1450. In this regard, second opening 1441b within post 1444 may be angled relative to opening 1451 in shaft body 1450 such that a segment of lock line 102 is trapped between post 1444 and shaft body 1450. For example, lever body 1445 may be rotated 90 degrees relative to shaft body so that lock line 102 may be trapped and compressed along an arc subtending an angle of about 90 degrees and second opening 1441b is oriented orthogonal to opening 1451. However, in other embodiments, lever body may be rotated to angles less than or greater than 90 degrees, such as 45 degrees to 135 degrees, for example. This may secure second end portion 102b of lock line 102 to lock handle shaft 1440 to prevent it from being decoupled from fixation device 112 until desired. Although it has been described that shaft body 1450 includes a female post opening and lever arm 1442 includes a male post, it is also contemplated that shaft body 1450 may include the male component, while lever arm may include the female component. In other words, shaft body 1450 may include a post, like that of post 1444, with a lock line opening extending therethrough, while lever arm 1442 may include a post opening, like that of opening 1444, and a lock line opening, such as opening 1441a, intersecting such post hole. Lever arm 1442 may be provided with a readily identifiable color as compared to other features of lock control assembly 1400 so as to identify lever arm 1442 with fixation device deployment in some examples. Such color may be magenta, for example.

As illustrated in the example of FIGS. 45C and 49F, lock handle shaft 1440 may extend through lock knob insert 1420 and lock knob 1410 so that lever arm 1442 extends from lock knob 1410 and first end portion 1452 of shaft body 1450 extends from insert 1420 so that it can be received with spool 1460. Teeth 1454 of shaft body 1450 may mesh with the teeth or splines of insert 1420 so as to rotationally constrain shaft body 1450 relative to insert 1420 and allow them to rotate together in some examples. Lock handle shaft 1440 may be secured to lock knob 1410 via a hairpin clip 1430 which may be inserted between knob 1410 and knob cap 1412 and engage a circumferential groove 1458 in shaft body 1450 according to various examples. In this arrangement, lever arm 1442 may be moved between the locked and unlocked configuration to correspondingly secure and release second end portion 102b of lock line 102. Abutment feature 1457 may limit rotation of lever arm 1442 so that when abutment feature 1457 stops the rotation of lever arm 1442, it is in the unlocked configuration, according to various examples of the disclosure.

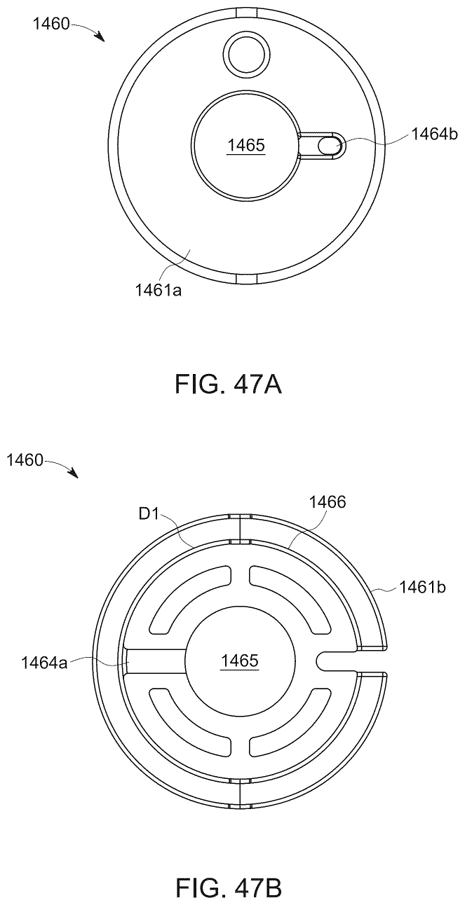

FIGS. 45B, 47A, and 47B depict a spool 1460 according to one example. Spool 1460 generally includes a first flange 1461a, a second flange 1461b, and a core 1466 extending between each flange 1461a, 1461b. Core 1466 may be cylindrical and may define a track along which lock line 102 may at least partially extend and may be wound during operation. In the embodiment depicted, core 1466 has a diameter D1. Spool 1460 may also have a shaft opening 1465 extending through core 1466 for receipt of first end portion 1452 of lock handle shaft 1450, as shown in the example of FIG. 45B. In operation, rotating spool 1460 in a first direction via lock knob 1410, winds lock line 102 about spool 1460 to thereby increase tension in lock line 102 and unlock a lock, such as locks 500 and 600, for example, and as described above. Conversely, rotating spool 1460 in an opposite second direction releases tension on lock line 102 and allows the lock to transition to the locked configuration.

Additionally, spool 1460 may include a notch 1462 which may be configured to receive fin 1455 of shaft body 1450 for transmission of torque from shaft body 1450 to spool 1460. Spool 1460 may also include a first lock line opening 1464a which may be aligned with notch 1462 and may extend through core 1466 of spool 1460, as best shown in the example of FIGS. 24C and 47B. This may allow first end portion 102a of lock line 102 to pass through notch 1456 in shaft body 1450 and through first lock line opening 1464a so that it can be partially wrapped around spool 1460 and extend through opening 1317 in housing 1310 to delivery catheter 1020, as shown in FIG. 45B. In one example, spool 1460 may include a second lock line opening 1464b which may extend through core 1466 of spool 1460 and through first flange 1461a, as shown in FIGS. 45B and 47A. Second end portion 102b of lock line 102 returning from delivery catheter 1020 may extend through second lock line opening 1464b out of spool 1460 and into opening 1451 of lock handle shaft 1440, as shown in FIG. 45B. First and second lock line openings 1464a, 1464b may be offset from each other 180 degrees about spool 1460.

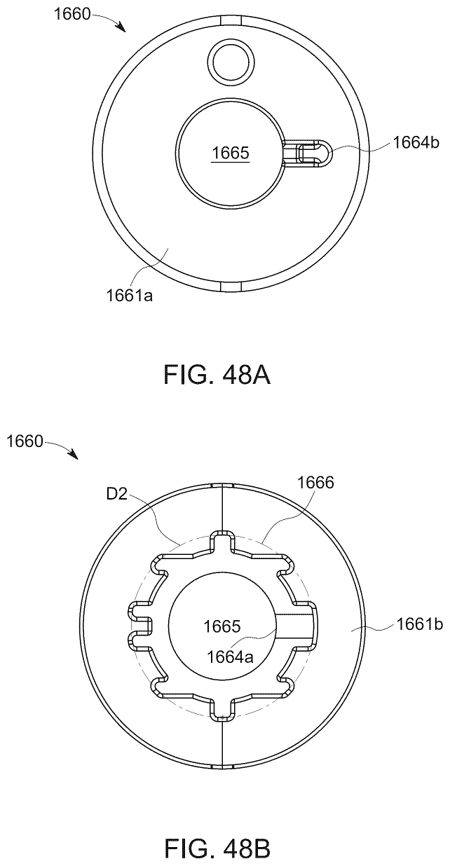

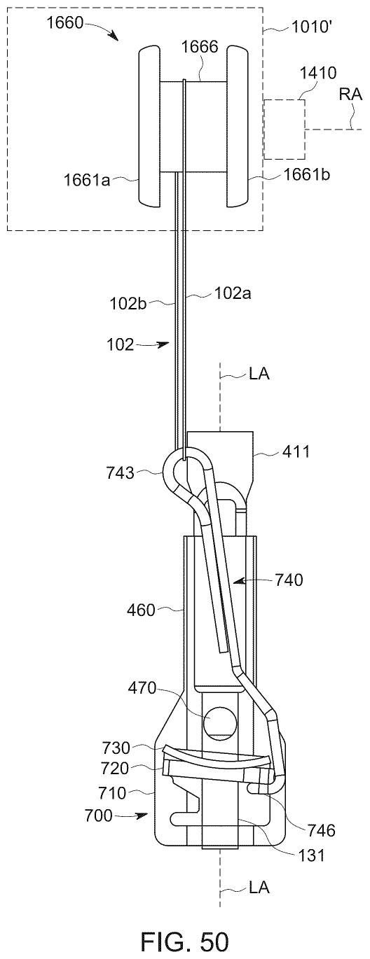

FIGS. 48A and 48B depict a spool 1660 according to another example. Spool 1660 is similar to spool 1460 in that it includes a pair of opposing flanges 1661a, 1661b and a core 1666 extending therebetween. Spool 1660 may also include first and second lock line openings 1664a, 1664b for receipt of first and second end portions 102a, 102b of lock line 102, as described with respect to spool 1460. However, core 1666 of spool 1660 has a diameter D2 which is smaller than diameter D1 of spool 1460. For example, diameter D2 may be approximately 20% smaller than diameter D1 of spool 1460. In one example, diameter D1 of spool 1460 may be about 0.560 in. (14.224 mm), and a diameter D2 of spool 1660 may be about 0.36 in. (9.144 mm) to 0.50 in. (12.7 mm), but preferably 0.445 in. (11.303 mm). As described below with respect to FIGS. 50 and 36, the smaller diameter D2 of spool 1660 unexpectedly helps limit the maximum force that may be applied to a lock, such as locks 500, 600, and 700, for example, which may be particularly beneficial for a SSH like that of harness 740 to reduce deformation thereof. The 0.445 in. diameter of spool 1660 creates a mechanical advantage ratio that limits peak tension. The relationship between spool diameter, lock line tension, and input torque follows the equation T=F*r, where T is torque, F is force, and r is radius. The 20% reduction in diameter creates not only a proportional change in mechanical advantage but also modifies the force-displacement curve. This diameter creates a balance in which sufficient mechanical advantage exists to generate the 1.25 to 3.0 lbf unlocking threshold while the physical constraints of the system naturally limit maximum force to approximately 6 lbf regardless of input torque. This self-limiting characteristic is further enhanced by the ergonomic constraints of knob 1410, which naturally limits the maximum torque a typical operator can apply.

As mentioned above, lock handle housing 1310 may also be fluid chamber of fluid management assembly 1300. In some examples, spool 1460 (or spool 1660) may be disposed within internal volume 1318 of lock handle housing 1310 such that it is aligned with lock handle extension 1350 extending therefrom, as shown in FIG. 45B. As mentioned above, spool 1460 can be rotationally fixed relative lock handle shaft 1440, for example, by a keying feature, such as fin 1455 and notch 1462. Accordingly, rotation of lock knob 1410, which can cause rotation of lock handle shaft 1440, can thereby cause rotation of spool 1460 and therefore increase or decrease tensile load on lock line 102 by way of lock line 102 spooling or unspooling around spool 1460. Lock handle housing 1310 can include a dowel pin 1319a in one example. First end portion 102a of lock line 102 can be routed around dowel pin 1319a and fin 1319b in lock handle housing 1310. This can align first end portion 102a of lock line 102 with core 1466 of spool 1460 and can manage slack in first end portion 102a of lock line 102. If slack is not managed, first end portion 102a of lock line 102 can disengage from core 1466 and from spool 1460. As noted above, first end portion 102a and second end portion 102b of lock line 102 may extend through opening 1317 in housing 1310 so as to extend to and from delivery catheter 1020, respectively. In one example, first end portion 102a can be routed around dowel pin 1319a and relative fin 1319b and routed at least partially around spool 1460 along core 1466 to first lock line opening 1464a where it passes through spool 1460 and enters opening 1451 of lock handle shaft 1140 via notch 1456. First end portion 102a may be fixedly secured to shaft body 1450 in one example. Additionally, in some examples, second end portion 102b may be routed around dowel pin 1319a, through second lock line opening 1464b in spool 1460, and through opening 1451 in lock handle shaft 1440 where it may be releasably secured via lever arm 1442.

Delivery system 1000 can be flushed, for example, via port 1302a in cover 1314 of lock handle housing 1310. For example, flush fluid can enter through port 1302a into the lock handle housing 1310 and out through opening 1317 into connector 1330. During preparation of system 1000, air can exit delivery system handle 1010 through second port 1302b of connector 1330 in some examples.

In one example, lock handle extension 1350 may rotatably receive lock handle shaft 1440 of the lock line handle 1402 and cylindrical body 1422 of lock knob insert 1420. As mentioned above, lock control assembly 1400 may include a locking system 1404 to limit movement of lock line handle 1402 relative delivery system handle 1010 and removal of lock line handle 1402 relative handle 1010. Lock system 1404 can be a latch-detent lock in one illustrative example. For example, the latch can include a distal U-lock 1470 and a proximal U-lock 1480 coupled to one another (and collectively referred to as the “U-lock” or “lock body”) and configured to surround lock handle extension 1350, as shown in FIGS. 45B and 45C. Proximal U-lock or first lock member 1480 can include a pin 1482 which can engage detents 1423 on lock knob insert 1420, when pin 1482 is insert through window 1352 disposed in lock handle extension 1350. In addition, as mentioned above, channel 1424 may intersect detents 1423. Thus, pin 1482 may also engage channel 1424 when pin 1482 is not engaging detents 1423. This helps prevent lock knob insert 1420 and the rest of lock handle 1402 from being inadvertently removed when rotating handle 1420 between the different positions (i.e., locked, unlocked, and third position described below). Distal U-lock or second lock member 1470 can include a rail 1472 to engage channel 1424 on lock knob insert 1420 according to one example.

In one example, locking system 1404 can further include a delivery handle insert 1490 and a release slider 1494, as shown in FIG. 45C, which can be used to release lock knob 1410. Delivery handle insert 1490 can include an indication, for example, an icon (such as a lock) or writing, to indicate that the delivery handle insert 1490 and release slider 1494 are intended to lock the lock line handle 1402. For example, and not by way of limitation, release slider 1494 can include a pin 1496 that can engage proximal U-lock 1480 (for example, through a window 1491 in delivery handle insert 1490). A spring 1484 or other biasing mechanism can be supported on pin 1354 of lock handle extension 1350 and can bias the latch into the locked position, for example. That is, spring 1484 can bias the latch in a distal direction to cause pin 1482 to engage detents 1423. Engagement of pin 1482 with detents 1423 can provide feedback, for example, audible and/or tactile, for a user that lock knob 1410 has reached a specific position (e.g., lock, unlock, override (also referred to herein as the third position or troubleshooting position)). Pin 1482 and detents 1423 can be arranged such that lock knob 1410 will not move from a specific position (e.g., lock, unlock, override), however, a user can move lock knob 1410 (i.e., overcome the engagement between the pin 1482 and the detents 1423) by turning knob 1410, and without touching release slider 1494. A portion of distal U-lock 1470, for example, rail 1472 on distal U-lock 1470 and channel 1424 can be arranged such that lock line handle 1402 can be removed from delivery handle 1010 when distal U-lock 1470 is disengaged from channel 1424 of knob insert 1420. For example, a user can slide release slider 1494 to disengage distal U-lock 1470 from channel 1424 and thereby release lock line handle 1402. For example, sliding release slider 1494 in the proximal direction can disengage rail 1472 on distal U-lock 1470 from channel 1424 and thereby permit the user to pull lock line handle 1402 from delivery handle 1010.

In an exemplary operation, fixation device 112, 112′, or 112″ can be delivered in a locked position (or first position). Lock knob 1410 can be in the locked position, which is shown for example, in FIGS. 42A and 49A. In the locked position, lock line 102 can have no (or relatively low) tension and lock knob 1410 can point distally. Spring 1484 can bias distal U-lock 1470 and proximal U-lock 1480 in a distal direction and therefore bias pin 1482 to engage a first detent 1423a of lock knob insert 1420 (FIG. 49A).

During one example of a procedure, the user can turn the lock knob 1410 to the unlocked position (or second position) to apply tension (or relatively higher tension) to lock line 102. Turning lock knob 1410 can overcome the engagement of pin 1482 with detent 1423. For example, lock knob 1410 can be rotated in a clockwise rotation (as viewed in FIG. 42A) such that lock knob 1410 can point upwardly (FIG. 49B). In such a position, lock knob 1410 itself can indicate to the user that fixation device 112 is in the unlocked position. Rotating lock knob 1410 can cause spool 1460 to rotate (as described above) and can increase tension on lock line 102 as lock line 102 spools around spool 1460. Such tensioning may tension both first and second portions 102a, 102b of lock line 102 so as to have an even pull on lock 500, 600, or 700. As noted above, increasing tension on lock line 102 can apply a force on harness 540, 640, or 740 which can unlock fixation device 112, 112′, or 112″, respectively, as described above. Spring 1484 can bias distal U-lock 1470 and proximal U-lock 1480 in a distal direction and therefore bias pin 1482 to engage a second detent 1423b of lock knob insert 1420 (FIG. 49B) and bias rail 1472 on distal U-lock 1470 to engage channel 1424 of lock knob insert 1420. Detents 1423, such as first and second detents 1423a, 1423b, (as well as labeling) can define the locked and unlocked positions. A click sound can be made as pin 1482 engages second detent 1423b. Detent 1423b can also prevent lock knob 1410 from returning to the locked position (until a user engages lock knob 1410).

In one example, the user can lock fixation device 112 by rotating lock knob 1410 in the counterclockwise direction (as viewed in FIG. 21A) to return lock knob 1410 to the locked position, unspool lock line 102 from spool 1460, and decrease tension on lock line 102. A click sound can be made as pin 1482 re-engages first detent 1423a. Rail 1472 can remain engaged with channel 1424 even as handle 1402 moves between the locked, unlocked, and, optionally, a third position according to an example of the disclosure. In one example, lock knob 1410 can be rotated beyond the unlock position (see e.g., FIG. 49C), to a third position corresponding to a third detent 1423c (see FIG. 49B), and to thereby further increase tension on lock line 102. This can be useful during certain procedures to ensure that fixation device 112, 112′, or 112″ properly unlocks. In this regard, the third position can be considered an override or troubleshooting position which may be used in the event the second position corresponding to second detent 1423b is not effective to unlock lock 500, 600, or 700. In various examples, additional detents 1423 can be provided to engage pin 1482 and hold lock knob 1410 in further rotated positions. For example, a fourth position can correspond to a fourth detent 1423d (see FIG. 49B) and may provide an additional override position. Rail 1472 can remain engaged with channel 1424 even as knob 1410 moves between the locked, unlocked, and additional positions. Although described in a particular arrangement, the third position, and any further positions, can include any suitable arrangement.

Before fixation device 112 can be deployed from delivery system 1000, lock line 102 may be decoupled from fixation device 112. To remove lock line 102, in one example, the user can ensure that lock knob 1410 is in the locked position. The user can also rotate lever arm 1442 of lock handle shaft 1440 to the unlocked position and retract release slider 1494 (i.e., the release slider 1494 can be pulled in the proximal direction). This can pull proximal U-lock 1480 (for example via pin 1496) and distal U-lock 1470 in a proximal direction (FIGS. 49D and 49E), which can disengage rail 1472 of distal U-lock 1470 from channel 1424. The user can pull lock line handle 1402 off delivery handle 1010 (FIG. 49F). Withdrawing lock line handle 1402 while lever arm 1442 is in the locked position can release second end portion 102b of lock line 102 from within lock handle shaft 1440, while first end portion 102a remains fixed to lock handle shaft 1440. Accordingly, in this example, withdrawing lock line handle 1402 pulls one end portion of lock line 102 (i.e., first end portion 102a), while the other end portion (i.e., second end portion 102b) is free to travel distally along the length of shaft 1440, through fixation device 112, and proximally along the length of delivery catheter 1020. Therefore, pulling lock line handle 1402 away from delivery handle 1010 withdraws lock line 102 from delivery system 1000 and fixation device 112.

If first end portion 102a of the lock line 102 becomes stuck, the user can disassemble lock line handle 1402 (FIG. 49G). For example, the user can remove hairpin clip 1430 and remove lock knob 1410 and lock knob insert 1420 from lock handle shaft 1440. This can provide direct access to first end portion 102a which is attached to lock handle shaft 1440. The user can pull second end portion 102b of lock line 102 to pull to free the “stuck” first end portion 102a. As described, lock control assembly 1100 is configured to provide discrete binary lock and unlock positions with tactile feedback which helps the clinician know when the locked and unlocked conditions are achieved. Additionally, lock control assembly 1100 with lock knob 1410 controls and limits the forces exerted on fixation device 112 thereby protecting its components from aggressive end user use and high lock line tension loads. Other exemplary lock control assemblies that may be implemented in interventional system 5 are disclosed in U.S. Pat. No. 11,622,859, the disclosure of which is hereby incorporated by reference herein in its entirety.

FIG. 50. illustrates the use of a delivery system handle 1010′ in conjunction with lock 700. Delivery system handle 1010′ is the same as delivery system handle 1010 with the exception that it includes spool 1660. In operation, lock line 102 extends from spool, through loops 743 of harness 740, and back to spool 1660. As shown, a longitudinal axis LA may be coaxial with stud 131, coupling member 460, and delivery systems shaft 411. Lock line 102 extends through loops 743 at a first side of longitudinal axis LA, while foot 746 of harness 740 is positioned at an opposite second side of longitudinal axis LA. As described above with respect to handle 1010, lock 700 may be unlocked by rotating spool 1660 about a rotational axis RA to a second detent, such as detent 1423b. Rotational axis RA may be approximately perpendicular (±5 degrees) to the tension force applied to lock line 102. Rotational axis RA may also be substantially perpendicular to longitudinal axis LA (±5 degrees). In circumstances in which rotating to second detent 1423b does not unlock lock 700, lock knob 1410 may be rotated to a third detent, such as third detent 1423c, to apply further tension on lock line 102 and for troubleshooting. As lock line 102 is tensioned, foot 746 engages binding plate 720 and raises it against the bias of biasing element 730 to unlock lock 700. As described above with respect to FIGS. 24A and 24B, harness 740 moves axially and has a limited runout RO2 as compared to harness 640.

Delivery system handle 1010′ confers multiple benefits particularly in relation to lock 700 and the SSH 740 thereof. In this regard, harness 740 may be more prone to deformation than harness 640 due to only a single foot 746 carrying the lock line load, whereas harness 640 is a DSH that distributes lock line tension between both feet 646a, 646b. Lock control assembly 1400 helps limit the peak force that may be applied to lock line 102 and thereby to harness 740. For example, the rotation of knob 1410 about rotational axis RA facilitates ease of control and avoids lock line slack which can result in rapid increases in force when slack is taken up, which is referred to herein as “slack line jumping.” This is in comparison to prior art devices which may utilize axially actuated levers that are pulled axially to unlock a fixation device. An example of such axially actuated levers are disclosed in U.S. Pat. No. 7,563,267. Such axially actuated levers are susceptible to slack line jumping and being pulled with too much force by aggressive use. Furthermore, the use of detents, such as second, third, and fourth detents 1423a, 1423b, 1423c, in conjunction with lock knob 1410 creates stop positions that helps prevent knob 1410 from being inadvertently over-torqued which can further help limit the peak load that may be imposed on lock line 102.

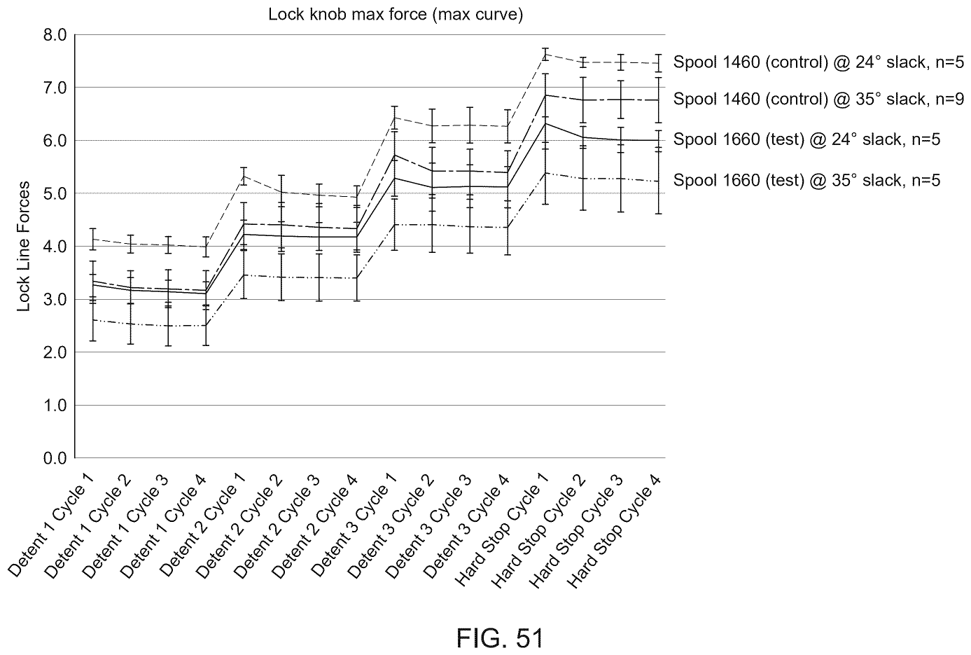

Additionally, spool 1660 is complementary to lock 700 and particularly to harness 740 in that it provides a balance between sufficient unlocking force to prevent inadvertent unlocking while limiting the peak load imposed on harness 740 to prevent undesirable deformation or failure of harness 740. In other words, lock 700 may be configured to have a threshold unlocking force. For example, lock 700 may have a threshold unlocking force of 1.25 lbf to 3.0 lbf. This range of the threshold unlocking force ensures that lock 700 has sufficient resistance to unintended unlocking when lock line 102 is removed by the user and that excessive lock line tension, which could damage harness 740, is not needed to unlock lock 700. Spool 1660 is configured to consistently provide such threshold locking force. Furthermore, as mentioned above, harness 740 may be more prone to deformation than harness 640. The reduced diameter D2 as compared to D1 unexpectedly reduces peak load imposed on lock line 102 and harness 740 by approximately 20%. For example, spool 1660 may deliver a peak load to lock line 102 of approximately 7.5 lbf, whereas spool 1460 may delivery a peak load of 6 lbf. This is illustrated in the line graph of FIG. 51 which illustrates the results of a test comparing the lock knob max force between the control (i.e., spool 1460) and the test (i.e., spool 1660) at various intervals defined by detents of handle 1010 and 1010′ (e.g., first, second, and third detents 1423a, 1423b, 1423c). As shown, spool 1660 provides sufficient threshold unlocking force while minimizing peak load at every load interval thereby ensuring consistent locking and unlocking while protecting harness 740. It is also noted that, while spool 1460 may have a higher peak load than spool 1660, spool 1460 provides similar benefits with respect to the DSH 640 of lock 600 in that spool 1460 in conjunction with locking assembly 1400 limits peak load and ensures consistent application of controlled tension.

Another unexpected result that is illustrated in FIG. 51 is that spool 1660 reduced the standard deviations of the lock knob max force and reduced the overall slope of the lock knob max force as compared to spool 1460 which helps ensure a more controlled locking operation and to limit excessive tensioning of the lock line.

### End Spool

Delivery catheter 1020 may include a proximal end portion 1021 and a distal end portion 1022, as best shown in FIG. 42B. As mentioned above proximal end portion 1021 of delivery catheter 1020 may be connected to delivery device handle 1010 and, in particular, may be coupled to connector 1330 of fluid delivery assembly 1300. Delivery catheter 1020 may include a plurality of lumens 1027 (see FIG. 16) extending from proximal end portion 1021 to distal end portion 1022. Each lumen may receive one of first proximal element line 101a, second proximal element line 101b, actuator rod 170, and first and second end portions 102a, 102b of lock line 102 which each extend through distal end 1022 of delivery catheter 1020 and to fixation device 112 where they are respectively coupled to proximal elements 140, stud 131 or base 139, and lock 300. Distal end 1022 of delivery catheter 1020 may include shaft 111 extending distally therefrom for coupling to fixation device 112. As discussed above, actuator rod 170 may extend through shaft 111 and into fixation device 112 where it may be coupled to stud 131 or base 139. Distal end 1022 of delivery catheter 1020 may optionally include a nose 1024. In the embodiment depicted, nose 1024 has a flanged shape. Such a flanged shape prevents nose 1024 from being retracted into a guide catheter of steerable guide system 5. However, it may be appreciated that nose 1024 may have any shape including bullet, rounded, blunt or pointed, to name a few.

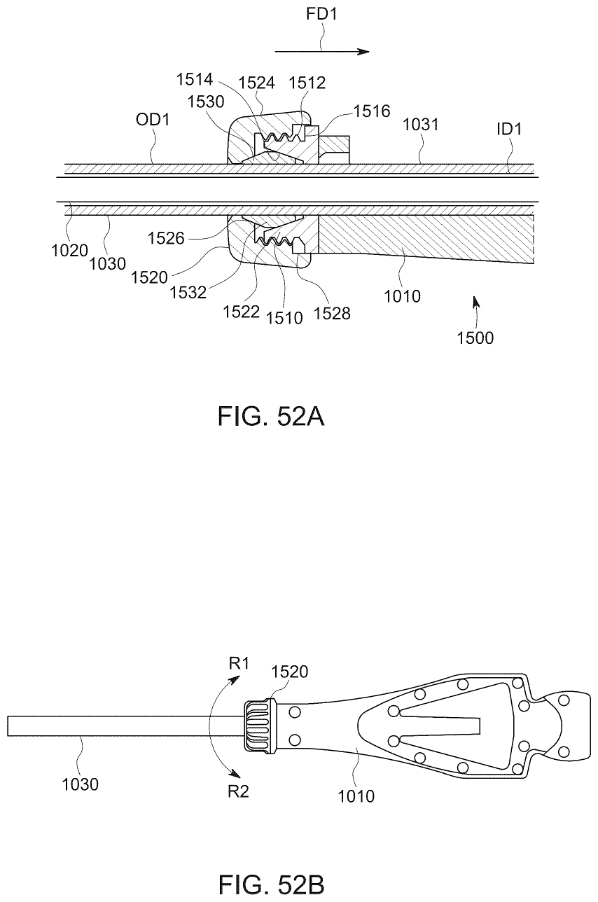

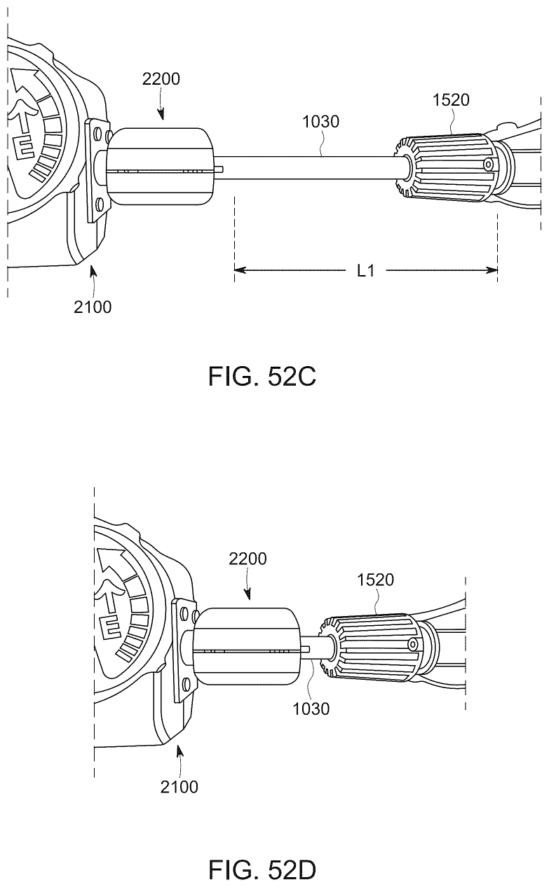

During a transcatheter procedure it may be desirable to advance and/or retract delivery catheter 1020 relative to steerable guide system 5 and to secure delivery catheter 1020 in a desired position relative to the target valve. FIGS. 42B and 52A-52D depict a delivery catheter fastening assembly 1500 according to an embodiment of the present disclosure and which may be configured to selectively secure delivery catheter 1020 from translation and release delivery catheter 1020 for proximal-distal translation. In the embodiment depicted, delivery catheter fastening assembly 1500 may generally include delivery catheter 1020, a brake shaft 1030, a distal end portion 1510 of delivery system handle 1010, a fastener knob 1520, and a compression ring 1530.

Brake shaft or sleeve 1030 may, in one example, have a first end portion or proximal end portion 1031 that that may extend through a distal opening of delivery system handle 1010 and may be slidable relative thereto, as best shown in FIGS. 42B and 52A. Brake shaft 1030 may also include a second end portion or distal end portion 1032 that may be fixedly secured to an inner guide catheter handle 2100 of steerable guide system 5, as shown in FIGS. 52C and 52D and as described in more detail below. Brake shaft 1030 may comprise an outer diameter OD1 and an inner diameter ID1. Delivery catheter 1020 may slidably and rotatably extend through brake shaft 1030 and inner diameter ID1 thereof, as shown in FIGS. 42B and 52A.

Distal end portion or static member 1510 may be disposed at a distal end of handle 1010 and may define the distal opening through which brake shaft 1030 and delivery catheter shaft 1020 extend. In this regard, proximal end portion 1031 of brake shaft 1030 may extend through distal end portion 1510 such that distal end portion 1510 circumferentially extends about outer diameter OD1, as shown in FIG. 52A. Distal end portion 1510 may include external threads 1512 and an inner engagement section 1514. Inner engagement section 1514 may be an inner sloping surface or chamfered portion that may taper radially inwardly toward brake shaft 1030 in a distal to proximal direction. Distal end portion 1510 may also have a stop surface 1516 disposed proximal of external threads 1512.

Fastener knob or dynamic member 1520 may be a threaded ring that may circumferentially extend about proximal end portion 1031 of brake shaft 1030 and may comprise internal threads 1522 that may be configured to cooperate or matingly engage with external threads 1512 of distal end portion 1510 of delivery system handle 1510, for example. Fastener knob 1520 may comprise an inner engagement section 1526 that may be an inner sloping surface or chamfered portion that tapers radially outwardly away from brake shaft 1030 in a distal to proximal direction. An outer surface 1524 of fastener knob 1520 may be tapered and/or may comprise textured or frictional features that may enhance grip. Additionally, fastener knob 1520 may have a stop-forming surface 1528 disposed proximal of internal threads and which may correspond to stop-forming surface 1516 of distal end portion 1510 which, when engaged, prevent further translation of fastener knob 1520 in a fastening direction FD1. This has the benefit of providing an indication to a practitioner that fastener knob 1520 has been adequately engaged with distal portion 1510 so as to reliably arrest translation of brake shaft 1030 and delivery catheter 1020.

Compression ring 1530 may circumferentially extend about outer diameter of brake shaft 1030 such that compression ring 1530 may come into direct contact with outer diameter OD1, as shown in the example of FIG. 52A. Compression ring 1530 may also be disposed between respective inner engagement sections 1514, 1526 of distal end portion 1510 and fastener knob 1520. Compression ring 1530 may comprise any suitable material including those with increased frictional properties. Such materials may include but are not limited to elastomeric or polymeric materials including medical-grade polymers, metal materials, ceramic materials, combinations thereof, or otherwise. Compression ring 1530 may define an outwardly bulging profile 1532 such that bulging profile 1532 generally peaks at a mid-section of compression ring 1530 and tapers inwardly towards its respective proximal and distal ends, according to one example. In this regard, with compression ring 1530 positioned between engagement sections 1514, 1526 of distal end portion 1510 and fastener knob 1520, inner sloping surfaces of distal end portion 1510 and fastener knob 1520 correspondingly engage bulging profile 1532, as illustrated in FIG. 52A.

During a transcatheter procedure it may be desirable to advance or retract delivery catheter 1030 to position fixation device 112 within a target valve. In one example, this may be achieved by unlocking fastening assembly 1500 by rotating fastener knob 1520 in a first rotation direction R1, as shown in FIG. 52B. Rotating knob 1520 in direction R1 advances knob 1520 in a distal direction which relieves compression on compression ring 1530 and correspondingly friction on brake shaft 1030. When the friction is sufficiently reduced, delivery system handle 1010 may be translated in a proximal or distal direction along brake shaft 1030 which correspondingly advances delivery catheter 1020 proximally or distally through brake shaft 1030, as illustrated in FIGS. 52C and 52D. As handle 1010 is advanced distally, proximal end portion 1031 of brake shaft 1030 is moved further proximally within handle 1010. Conversely, as handle 1010 is moved proximally, proximal end portion 1031 of brake shaft 1030 moves toward distal end portion 1510 of fastening assembly 1500. As shown in FIG. 52C, the maximum travel length of handle 1010 relative to brake shaft is L1. This length L1 may be limited so as to prevent fixation device 112 from advancing too far and potentially damaging the heart. For example, in a transfemoral approach to a mitral valve or tricuspid valve, fixation device 112 may be initially positioned within an atrium and advanced toward the valve from the atrium. L1 may be limited so as to not overshoot valve and damage the chordae or ventricle. L1 may be about 3 to 4 inches, for example.

Once fixation device 112 is in a desired position relative to the target valve, fastening assembly 1500 may be locked by rotating fastener knob 1520 in a second rotation R2 direction opposite first rotation direction R1, as shown in the example of FIG. 52B. As fastener knob 1520 is rotated about distal portion 1510 of delivery handle 1010, fastener knob 1520 may translate in a longitudinal fastening direction FD1, which may be the proximal direction. As fastener knob 1520 is translated in the fastening direction FD1, engagement sections 1514, 1526 of distal end portion 1510 and fastener knob 1520 compress bulging profile 1532 of compression ring 1532 which correspondingly compresses compression ring 1532 against outer diameter OD1 of brake shaft 1030 until the friction resulting from such compression is sufficient to arrest proximal-distal translation of handle 1010 relative to brake shaft 1030. Stop-forming features 1516, 1528 of distal end portion 1510 and fastener knob 1520 may, for example, provide an indication that arrest has been achieved. By providing integrated fastening system 1500, superfluous floating components, which in practice are easily and frequently lost, are minimized, as fastener knob 1520 in cooperation with distal end portion 1510 of the delivery system handle 1010 provide a simplified, effective, and intuitive system for restricting or arresting translation of delivery catheter 1020 relative to brake shaft 1030 and correspondingly steerable guide system 5. Additionally, fastener knob 1520 improves ergonomics and ease of repeated and precise use by the practitioner by providing a conveniently sized and located component for effectively arresting translation. Other delivery catheter fastener examples that may be implemented in delivery system are disclosed in '419 Publication mentioned above and are incorporated herein by reference.

FIGS. 41, 53, 54A-54E, 55, and 56A-41B depict steerable guide system 5 according to an embodiment of the present disclosure. Steerable guide system 5 may be configured to guide delivery catheter 1020 and fixation device 112 coupled thereto percutaneously through the cardiovascular system to a position near the target valve. In the embodiment depicted, steerable guide system 5 generally includes an inner guide catheter assembly 2000 and an outer guide catheter assembly 3000. Inner guide catheter assembly 2000 may generally include one or more of an inner guide catheter handle 2100 and an inner guide catheter 2300 extending distally therefrom, and outer guide catheter assembly 3000 may generally include one or more of an outer guide catheter handle 3100 and outer guide catheter 3300 extending distally therefrom.

Outer guide catheter 3300 may have a proximal end 3301, a distal end 3302, and a central lumen extending therethrough, and inner guide catheter 2300 may have a proximal end 2301, a distal end 2302, and a central lumen extending therethrough. Inner guide catheter 2300 may be positioned coaxially within the central lumen of outer guide catheter 3300, as shown in FIG. 41. Distal ends 2302, 3302 of catheters 2300, 3300, respectively, may be sized to be passable to a body cavity, typically through a body lumen such as a vascular lumen. Outer guide catheter 3300 and/or the inner guide catheter 2300 may be precurved and/or have steering mechanisms to position distal ends 2302, 3302 in desired directions.

Steering of outer guide catheter 3300 and inner guide catheter 2300 may be achieved by actuation of one or more steering mechanisms. Actuation of the steering mechanisms may be achieved with the use of actuators which may be located on inner and outer guide catheter handles 2100, 3100. Outer guide catheter handle 3100 may be connected to proximal end 3301 of outer guide catheter 3300 and may remain outside of a patient's body during use. Outer guide catheter handle 3100 may include one or more steering actuators or steering knobs 3102 which may be used to bend, arc, or reshape outer guide catheter 3300, such as to form a primary curve. Inner guide catheter handle 2100 may be connected to proximal end 2301 of the inner guide catheter 2300 and may optionally join with outer guide catheter handle 3100 to form one larger handle. Inner guide catheter handle 2100 may include one or more steering actuator 2102 which may be used to bend, arc, or reshape inner guide catheter 2300, such as to form a secondary curve and move or sweep distal end 2302 of inner guide catheter 2300 through one or more angles of motion.

Steerable guide system 5 may have different configurations depending on the approach taken to the target valve and may differ depending on which valve is targeted. For example, approaching a tricuspid valve through the inferior vena cava may involve tighter turns than approaching a mitral valve from the inferior vena cava and across the interatrial septum. Thus, the steering mechanisms and actuators 2102, 3102 of those steering mechanisms for inner and outer guide catheter assemblies 2000, 3000 may be configured accordingly. Examples of inner and outer guide catheter assemblies, steering mechanisms, and actuators thereof which may be incorporated into interventional system 3 and utilized to target a mitral valve are disclosed in U.S. Pat. No. 7,226,467, the disclosure of which is hereby incorporated by reference herein in its entirety. Also, examples of inner and outer guide catheter assemblies, steering mechanisms, and actuators thereof that may be incorporated into interventional system 3 and utilized to target a tricuspid valve are disclosed in U.S. Pub. No. 2023/0131595, the disclosure of which is hereby incorporated by reference herein in its entirety.

During a transcatheter procedure, it may be desirable to support and stabilize steerable guide system 5 and delivery system 1000. However, it may also be desirable to move certain components thereof in a controlled manner. In this regard, interventional system 3 may include, in some examples, one or more of a proximal attachment assembly or first attachment assembly 2200 and a distal attachment assembly or second attachment assembly 3200.

FIGS. 54A-54E depict one example of proximal attachment assembly 2200. Proximal attachment assembly 2200 may be positioned between inner guide catheter handle 2100 and delivery system handle 1010. Proximal attachment assembly 2200 generally includes one or more of a support frame 2210 and first and second engagement members 2240a, 2240b moveably attached to support frame 2210.

In the embodiment depicted, support frame or support body 2210 may include an upper portion and a lower portion extending from the upper portion. The upper portion may include a mount member 2220 which may be configured to mount or otherwise connect to inner guide catheter handle 2100, as shown in the example of FIGS. 54D and 54E. In the embodiment depicted, mount member 2220 may be in the form of a mounting plate which may be secured to inner guide catheter 2100 via one or more fasteners. However, in other embodiments, mount member 2220 may be connected to inner guide catheter handle 2100 via welding, for example, or mount member 2220 may be integral with inner guide catheter handle 2100 such as via an injection molding process, as another example.

The upper portion may, for example, also include a boss 2221 extending proximally from mount member 2220. Boss 2221 may define a first opening 2222 and a second opening 2223 which may each extend at least partially into boss 2221 and in a proximal-distal direction. First opening 2222 may be configured to receive brake shaft 1030 such that brake shaft 1030 may pass through proximal attachment assembly 2200 as it extends from delivery system handle 1010 to inner guide catheter handle 2100 where it is fixedly secured. However, in some embodiments, proximal attachment assembly 2200 may fixedly secure brake shaft 1030 to inner guide catheter handle 2100. Second opening or pin opening 2223 may be offset in an upward direction from first opening 2222 and may be generally parallel relative thereto. Second opening 2223 may be configured to receive a hinge pin 2204, as shown in the example of FIG. 54A.

The lower portion of proximal attachment assembly 2200 may include a central support wall or central member 2230, a transverse plate or transverse member 2232, and first and second fingers 2234a, 2234b. As shown in the example of FIGS. 54B and 54C, central support wall 2230 may extend downwardly from boss 2221 and may extend in a proximal-distal direction. Transverse plate 2232 may extend transverse to central support wall 2230, such as perpendicular (±5 degrees) relative thereto, and may be connected to a lower end of central support wall 2230 opposite an upper end thereof which may be connected to boss 2221, according to one example of the disclosure. First finger or first end wall 2234a and second finger or second end wall 2234b may extend from transverse plate 2232 in a downward direction and may be respectively disposed at proximal and distal ends of transverse plate 2232 so as to form a gap therebetween, for example. As shown in FIGS. 54B and 54C, fingers 2234a, 2234b each define a width W1 which extends in a direction transverse to the proximal-distal direction.

First and second engagement members 2240a, 2240b may be pivotably connected to support frame 2210 in one example. In this regard, first and second members 2240a, 2240b may, for example, be connected to each other via hinge pin 2204 which extends through boss 2221 of proximal attachment assembly, as mentioned above. Such pivotable connection may be made at or near respective first ends of engagement members 2240a, 2240b. As shown in the illustrated example, first and second engagement members 2240a, 2240b extend about boss 2221 and downwardly at opposite sides of central support wall 2230. Boss 2221 may have a tear-drop shaped profile which may correspond to a tear-drop shape opening defined by first and second engagement members 2240a, 2240b. This shape may facilitate pivotable action of first and second engagement members 2240a, 2240b about boss 2221. A spring 2250 may be positioned between first and second engagement members 2240a, 2240b such that the spring 2250 biases engagement members 2240a, 2240b toward a first position or locked configuration away from each other, as shown in FIG. 54D. In other words, when first and second engagement members 2240a, 2240b are in the first position, they may be in an expanded state. However, pressing on engagement members 2240a, 2240b against the bias of spring 2250 may move engagement members 2240a, 2240b to a second position or unlocked configuration in which they may abut each other, as shown in the example of FIG. 54E. Thus, when first and second members 2240a, 2240b are in the second position, they may be in a collapsed state according to one example of the disclosure.

Each engagement member 2240a, 2240b may, in various examples, also include a hook member or capture member 2242 extending downwardly from a second end thereof. Such capture members 2242 may each extend through transverse plate 2232 of support frame 2210 and within the gap between fingers 2234a, 2234b of support frame 2210. Thus, in one example of the first position, first and second engagement members 2240a, 2240b may engage transverse plate 2232 to limit or stop their outward expansion. Capture members 2242 may each include a sloping surface 2244 that slopes outwardly and terminates at a shoulder 2246 which faces transverse plate 2232, as best shown in the example of FIG. 54B. Each shoulder 2246 and transverse plate 2232 may define a slot or channel 2202 when first and second engagement members 2240a, 2240b are in the first position. Such slot 2202 may be configured to slidably receive a corresponding rail of stabilizer 4000, which is described in more detail below. Thus, in the example first position, capture members 2242 may extend beyond width W1 of fingers 2234a, 2234b. In other words, capture members 2242 in the first position can, in one example, collectively define a width greater than width W1 of fingers 2234a, 2234b. However, according to one example, when engagement members 2240a, 2240b are in the second position, as shown in FIG. 54C, capture members 2242 are either flush with or narrower than the width W1 of fingers 2234a, 2234b. Although it has been described that first and second engagement members 2240a, 2240b are each moveable relative to support frame 2210, it should be understood that in some embodiments one of such engagement members 2240a, 2240b may be static, while the other engagement member 2240a, 2240b may be dynamic. In other words, one of engagement members 2240a, 2240b may only be moveable relative to support frame 2210 between the first and second configurations (i.e., locked and unlocked configurations).

FIGS. 56A and 56B depict one example of distal attachment assembly 3200. Distal attachment assembly 3200 can be positioned distal of outer guide catheter handle 3100. Thus, in the embodiment depicted in FIG. 41, inner and outer guide catheter handles 2100, 3100 are positioned between proximal and distal attachments 2200, 3200. Distal attachment assembly 3200 may also have a snap-fit mechanism or the like. However, the snap-fit mechanism of distal attachment may be configured to constrain all movement when attached to stabilizer 4000. Distal attachment 3200 may generally include one or more of a bushing 3210, a housing 3220, and a connection mechanism for connecting to stabilizer 4000.

Bushing or shaft 3210 may define a central opening 3212 through which outer guide catheter 3300 may extend, as shown in the example of FIG. 56A. Bushing 3210 may extend into outer guide catheter handle 3100 and be connected to a distal end of outer guide catheter handle 3100 via a collar 3112 which may have a set screw that rotationally and translationally secures bushing 3210 according to various examples.

Housing or support body 3220 may, in one example, surround the portion of bushing 3210 extending from outer guide catheter handle 3100 and may include a lower housing portion 3220a and an upper housing portion 3220b. As shown in the example of FIG. 56B, one or more O-rings 3240 may be positioned between bushing 3210 and housing 3220. For example, distal attachment 3200 may have four O-rings 3240 with two O-rings 3240 located at a proximal location and two O-rings 3240 located at a distal position. This distribution of O-rings 3240 helps to ensure even distribution of load/compression. However, other configurations are contemplated, such as two or more O-rings 3240 distributed at even intervals along a length of bushing 3210. To help contain O-rings 3240, lower housing portion 3220a and bushing 3210 may have corresponding lips 3215, 3225 preventing proximal-distal travel of O-rings 3240. O-rings 3240 may be made from a material that enhances friction, such as silicon, for example. This arrangement helps provide rotational friction between housing 3220 and bushing 3210 and prevent unintentional rotation of outer and inner guide catheters 2300, 3300 while utilizing inner and outer guide catheter handles 2100, 3100. In other words, O-rings 3240 help provide constant friction to prevent unintentional rotation of inner and outer catheters 2300, 3300. This O-ring configuration also helps coaxially correct inner and outer guide catheters 2300, 3300 to help keep them coaxially aligned. Furthermore, O-rings 3240 help protect outer catheter 3300 by removing direct contact of catheter 3300 and by promoting handle torque transmission directly to O-rings 3240 rather than directly from handle 3100 to catheter 3300 in order to brake the system.

Lower housing portion 3220a may include a connection mechanism for connecting distal attachment 3200 to stabilizer 4000, for example. In the depicted embodiment, the connection mechanism may include a dynamic pin or first engagement member 3226a and a static pin or second engagement member 3226b, for example. Static pin 3226b may extend through a first opening 3224 in lower housing 3220a, while dynamic pin 3226a may extend through a second opening 3222 in lower housing 3220a, as best shown in the example of FIG. 56B. In one example, second opening 3222 may be an elongate opening or elongate slot allowing pin 3226a to dynamically move in a proximal-distal direction. Lower housing 3220a may include a chamber 3228 with a spring or other biasing element 3250 disposed therein, for example. Spring 3250 may bias dynamic pin 3226a in a first position or proximal position. When the spring bias is overcome, dynamic pin 3226a may move to a second position or second position which is closer to static pin 3226b than the first position. Thus, pins 3226a, 3226b have a first configuration when pin 3226a is in the first position and a second configuration 3226b when pin 3226a is in the second position, according to an example of the disclosure.

FIGS. 57A-57C depict stabilizer 4000 according to an embodiment of the present disclosure. Stabilizer 4000 may generally include one or more of a base 4010, one or more support arms 4020, a distal attachment 4030, and a proximal attachment 4040.

Base or platform 4010 may be a plate with a flat bottom for placement on a flat surface of a table or the like. Base 4010 may define a longitudinal axis A3 and may include one or more handles 4012 for manipulating stabilizer 400. For example, as shown in FIG. 57A, base 4010 may include one or more of handles 4012 at proximal and distal ends of base 4010.

Support arm 4020 may extend upwardly from base 4010 and may support both distal attachment 4030 and proximal attachment 4040, as shown in the example of FIG. 57A. However, in other embodiments, separate support arms 4020 may extend from base 4010 that may separately support distal and proximal attachments 4030, 4040.

Proximal attachment or first attachment 4040 may include one or more of a first rail or first member 4042a, a second rail or second member 4042b, a first end wall 4044a, and a second end wall 4044b. First and second rails 4042a, 4042b may extend parallel (±5 degrees) to each other between first and second end walls 4044a, 4044b and may each have a planar upper surface 4043 and planar lower surface (not shown) according to one example of the disclosure. End walls 4044a, 4044b and rails 4042a, 4042b may define an elongate slot 4046 that may be rectangular in shape, as shown in the example of FIG. 57C. However, elongate slot 4046 may also have other shapes, such as oval or pill-shaped, for example. Elongate slot 4046 may have a width slightly wider than width W1 of fingers 2234a, 2234b of proximal attachment 2200. Additionally, elongate slot 4046 may have a length L2 which may be defined from first end wall 4044a to second end wall 4044b. As explained in more detail below, this length L2 may correspond to a distance that inner guide catheter 2300 may translate relative to outer guide catheter 3300. In this regard, first and second end walls 4044a, 4044b form stop surfaces for proximal attachment assembly 2200 and correspondingly a translational limit for inner guide catheter handle 2100. Proximal attachment 2200 may also include proximal and distal tabs 4048a, 4048b extending downward therefrom, for example. Such tabs 4048a, 4048b may be used as leverage points for manual manipulation and translation of inner guide catheter handle 2100 when proximal attachment assembly 2200 is connected to proximal attachment 4040. In one example, proximal attachment 4040 extends along a longitudinal axis A4 which may form an acute angle relative to axis A3 of base 4010.

Distal attachment or second attachment 4030 may include a pair of attachment members 4032 and a recess 4031 extending therebetween. As shown in the example of FIG. 57B, each attachment member 4032 may include a first hook or first capture member 4034a and a second hook or second capture member 4034b. First capture member 4034a extends in a distal direction while second capture member 4034b extends in a proximal direction such that each capture member 4034a, 4034b defines a corresponding recess 4035a, 4035b for receipt of dynamic pin 3226a and static pin 3226b, respectively. Additionally, first capture member 4034a may include a cam surface 4038 that slopes in a proximal to distal direction. Such cam surface 4038 may be configured to deflect dynamic pin 3226a of distal attachment assembly 3200 from its first position to its second position thereby allowing dynamic pin 3226a to pass by first hook 4034a and into its corresponding recess 4035a where it returns to the first position or somewhere between the first and second positions. Each attachment member 4032 may also include an intermediate projection 4036 that defines sloped surface 4037 that is sloped toward second hook 4034b in various examples. This sloped surface 4037 may direct static pin 3226b of distal attachment assembly 3200 into recess 4035b. Attachment members 4032 may be laterally offset from each other and may extend parallel relative to each other (±5 degrees) so as to define recess 4031 which may be configured to receive lower housing portion 3220a of distal attachment assembly 3200 according to various examples of the disclosure.

Stabilizer 4000 may be made from a single sheet of metal material such that at least the base 4010, support arm 4020, and proximal attachment 4040 are stamped from the single sheet of metal material and bent into their depicted configuration according to one example of the disclosure. The single-sheet construction provides several advantages over multi-component assemblies, including enhanced structural integrity, reduced manufacturing complexity, and elimination of potential failure points at component junctions. The metal material may include, but is not limited to, stainless steel, titanium alloys, aluminum alloys, and the like. Distal attachment 4030 may be separately manufactured and connected to support arm 4020, such as by welding, for example. However, in other embodiments distal attachment 4030 may also be stamped out of the single sheet of metal material. The depicted configuration of stabilizer 4000 may reduce welding points, which may be potential points of failure, and may reduce cleaning and sterilization risks.

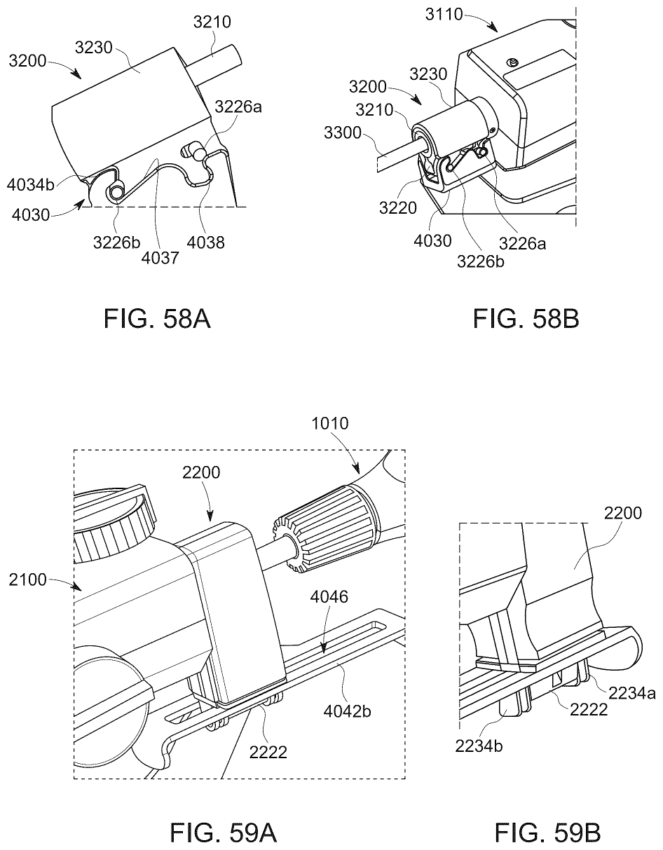

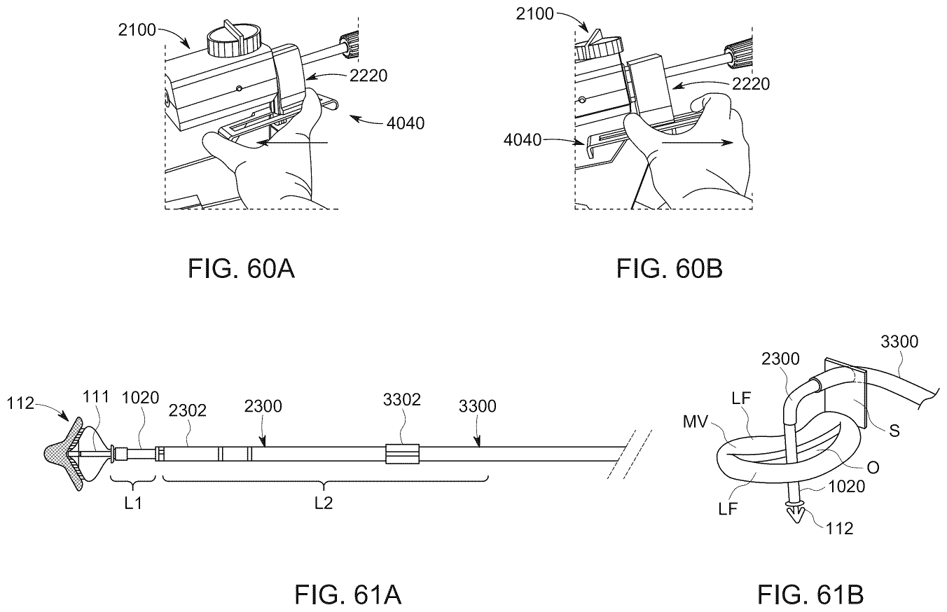

FIGS. 58A and 58B illustrate one example of the connection between distal attachment 4030 of stabilizer 4000 and distal attachment assembly 3200 of steerable guide system 5. In this regard, distal attachment assembly 3200 may be attached to distal attachment 4030 first before proximal attachment assembly 2200. This may be achieved by sliding static pin 3226b along sloped surface 4037 of intermediate projection 4036 and in the distal direction until pin 3226b is fully received within recess 4035b and captured by first hook 4034b, as illustrated in the example of FIG. 58A. At this point, distal attachment assembly 3200 may be rotated about first pin 3226b in a downward direction until dynamic pin 3226a contacts cam surface 4038. Further downward rotation causes cam surface 4038 to urge dynamic pin 3226a from its first position to its second position which clears first hook 4034a allowing dynamic pin 3226a to be received within recess 4035a. Once dynamic pin 3226a is received within recess 4035a, it returns to the first position or somewhere between the first and second positions thereby locking distal attachment assembly 3200 to distal attachment 4030 of stabilizer 4000, as illustrated in the example of FIG. 58B.

FIGS. 59A and 44B illustrate one example of the connection between proximal attachment 4040 of stabilizer 4000 and proximal attachment assembly 2200 of steerable guide system 5. In this regard, once distal attachment assembly 3200 is secured to distal attachment 4030, proximal attachment assembly 2200 may be lowered toward elongate slot 4046 of proximal attachment 4040. As proximal attachment assembly 2200 is lowered, fingers 2234a, 2234b and capture members 2242 are received within elongate slot 4046 such that rails 4042a, 4042b engage sloped surfaces 2244 of capture members 2242 and urge them inwardly from their first position to their second position in which their collective width is equal to or lesser than width of fingers 2234a, 2234b. However, since width W1 of fingers 2234a, 2234b is slightly less than that of elongate slot 4046, fingers 2234a, 2234b may advance into elongate slot 4046. Once rails 4042a, 4042b pass shoulders 2046, the bias of spring 2250 returns hook capture 2242 and corresponding engagement members 2240a, 2240b to their respective first position. In this configuration, first and second rails 4042a, 4042b are received in their corresponding slots 2022. Additionally, spring 2250 pushes capture members 2242 outwardly into contact with rails 4042a, 4042b with sufficient force so as to create friction between them which limits inadvertent movement of proximal attachment assembly 2200 along elongate slot 4046.

Although it has been described that proximal attachment assembly 2200 of the multi-catheter system includes moveable engagement members 2240a, 2240b that are configured to attach to rails 4042a, 4042b and translate within slot 4046 of proximal attachment 4040, it should be understood that this arrangement can be reversed. For example, stabilizer 4000 may have a proximal attachment with engagement members, like engagement members 2240a, 2240b, that are moveable between first and second positions, and the multi-catheter system may include a proximal attachment with rails and an elongate slot, like rails 4042a, 4042b and slot 40446, for receipt of such engagement members of the stabilizer. Similarly, the configurations of distal attachment assembly 3200 of the multi-catheter system and distal attachment 4030 can be reversed. Thus, stabilizer 4000 may have a distal attachment assembly with a static and dynamic pin, such as static and dynamic pins 3226a, 3226b, and the multi-catheter system may have a distal attachment with attachment members, like attachment members 4032 for receiving and engaging with the static and dynamic pins.

FIGS. 60A-61B illustrate the use of interventional system 5 to position fixation device 112 within a target valve, which is depicted as a mitral valve MV, but should be understood to alternatively be a tricuspid valve. In a transcatheter procedure for repairing mitral valve MV, distal end 3302 of outer guide catheter 3300 is advanced through the interatrial septum S and positioned within the right atrium proximal to mitral valve MV. To help position fixation device 112 above the desired location along the line of coaptation and within the valve orifice O, inner guide catheter 2300 may be advanced from outer guide catheter 3300. This may be achieved by sliding proximal attachment assembly 2100 distally (i.e., forward) within elongate slot 4046 of proximal attachment 4040, as shown in FIG. 60A. The practitioner may utilize distal tab 4048b as a leverage point to advance proximal attachment assembly 2100. Should it be determined that it was advanced too far, the practitioner may slide proximal attachment assembly 2100 proximally (i.e., backward) optionally utilizing proximal tab 4048a as a leverage point, as shown in FIG. 45A. As illustrated in FIG. 61A, the maximum length at which inner guide catheter 2300 can advance from outer guide catheter is length L2, which corresponds to the length of elongate slot 4046. This length L2 may be determined via a statistical analysis of heart geometries of a select patient population. For example, the dimensions of the left atria of the select population may be analyzed up to the first, second, or third standard deviation such that when inner guide catheter 2300 is advanced to the maximum length L2 from outer guide catheter 3300, fixation device 112 does not bump into and damage any structures of the atrium. Thus, elongate slot 4046 of proximal attachment 4040 forms a “parking spot” or “landing zone” in which, when proximal attachment assembly 2100 is as proximal as possible (i.e., abutting first end wall 4044a), fixation device 112 is positioned within and maintained within distal end 3302 of outer guide catheter 3300. This provides the operator with visual confirmation that fixation device 112 is sheathed so that fluoroscopic confirmation is not needed which helps reduce the overall time the patient and operator are exposed to fluoroscopy and enhances patient safety. As shown in FIG. 61B, inner guide catheter 2300 may also be curved so that fixation device 112 may be oriented toward mitral valve.

Once fixation device 112 is positioned above the desired location along the line of coaptation, delivery catheter 1020 may be advanced from inner guide catheter 2300 into the valve. This may be achieved by unlocking fastening assembly 1500 by rotating fastening knob 1520 in the first rotation direction R1, according to one example. Delivery system handle 1010 and delivery catheter 1020 may then be advanced along brake shaft 1030 until the desired position within valve MV is achieved, at which point fastening assembly 1500 may be locked by rotating knob 1520 in the second rotation direction R2. The maximum length at which delivery catheter 1020 can advance from inner guide catheter is length L1, which corresponds to the maximum distance handle 1010 can advance along brake shaft 1020. Again, this length can be determined from a population level analysis of heart geometries to set the L1 limit. From there, leaflets LF may be grasped. Once the leaflets LF have been sufficiently grasped and fixation device 112 is moved to the closed position, lock line 102 may be released, as described above. Additionally, deployment system 1170 may be disengaged from slider 1110 and actuator shaft 1140. Actuator shaft 1140 may then be rotated to release actuator rod 170 from stud 131 or base 139 and retracted which releases coupling member 160 from shaft 111 and also releases proximal element lines 101a, 101b from shaft 111, as also described above.

Although interventional system 3 has been described as including fixation device 112, delivery system 1000, steerable guide system 5, and stabilizer 4000, it should be understood that interventional system 3 can include more, less, or a different arrangement of components. For example, interventional system 3 may not include stabilizer 4000, which may be appropriate for procedures where manual stabilization is preferred or where limited procedure space precludes the use of a separate stabilizing apparatus. In another example of interventional system 3, steerable guide system 5 may be integrated into delivery system 1000 such that delivery catheter 1020 may itself be steerable via controls integrated into delivery handle 1010. This integrated approach could reduce the overall system profile and simplify the procedural workflow by eliminating separate catheter systems. In an even further example of interventional system 3, steerable guide system 5 may alternatively be configured such that a single guide catheter provides all the steering inputs to guide fixation device 112 to the target valve rather than inner and outer guide catheter assemblies 2000, 3000 for performing the same. This single-catheter configuration might be advantageous for specific anatomical approaches or in pediatric applications where space constraints are significant. In a still further embodiment, a robotically guided catheter (not shown) may be provided in interventional system 3 to both stabilize and steer delivery system 1000 and fixation device 112 to the target valve. The robotic system could incorporate haptic feedback mechanisms to provide the operator with tactile sensation during the procedure while offering enhanced stability and precision. However, in such robotically assisted procedure, a practitioner may still manually grasp leaflets and deploy fixation device 112 via the controls of delivery device system 1000.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. An implantable fixation device for fixating tissue comprising:
a first fixation element and a second fixation element, each of the first and second fixation elements having a first end, a free end opposite the first end, and an engagement surface therebetween for engaging tissue, the free ends being moveable between a first position and a second position;
an actuation mechanism coupled to the first and second fixation elements, the actuation mechanism having a stud configured to move in an axial direction, wherein movement of the stud in the axial direction moves the first and second fixation elements between the first and second positions; and
a lock comprising:
a wedging element having a first surface and an opposing second surface, the wedging element being at least partially disposed about the stud and being moveable between a first position in which the wedging element engages the stud to arrest its movement in the axial direction, and a second position in which the wedging element is disengaged from the stud allowing the stud to move in the axial direction,
a biasing element engaging the first surface of the wedging element and being configured to bias the wedging element toward the first position thereof, and
a harness having a first end portion and a second end portion, the second end portion having a single foot, the single foot being moveable to engage the second surface of the wedging element and move the wedging element against the bias of the biasing element from the first position to the second position of the wedging element,
wherein pulling the first end portion of the harness in the axial direction moves the wedging element to the second position and pivots the harness from a first orientation to a second orientation, the second orientation being 6 degrees or less from the first orientation.

2. The implantable fixation device of claim 1, wherein the first end portion of the harness has a first straight segment, and when the harness is in the first orientation, the first straight segment is parallel to a longitudinal axis of the stud.

3. The implantable fixation device of claim 2, wherein the first end portion of the harness has a second straight segment connected to the first straight segment and a loop disposed between the first and second straight segments, the loop defining an eyelet configured to receive a lock line.

4. The implantable fixation device of claim 3, wherein the first and second straight segments are connected to each other by at least three and up to five spot welds.

5. The implantable fixation device of claim 3, wherein the harness is formed from a wire, and the second straight segment of the first end portion defines a terminal end of the wire.

6. The implantable fixation device of claim 1, wherein the first end portion of the harness has a first straight segment, and the single foot is oriented at an acute angle relative to the first straight segment.

7. The implantable fixation device of claim 6, wherein the acute angle is 80 to 85 degrees.

8. The implantable fixation device of claim 7, wherein:
the distal end portion of the harness includes a first straight segment and a second straight segment, the first straight segment being angled outwardly relative to the first straight segment of the proximal end portion, and the second straight segment being parallel to the first straight segment of the proximal end portion, and
the single foot is connected to and extends inwardly from the second straight segment of the distal end portion.

9. The implantable fixation device of claim 1, wherein:
the wedging element is a binding plate having a first end, a second end, and an opening extending through the first and second surfaces of the wedging element,
the stud is disposed within the opening of the wedging element, and
the single foot is disposed adjacent to the second end of the wedging element such that, when the foot engages the wedging element, the foot pivots the second end of the wedging element about the first end of the wedging element.

10. The implantable fixation device of claim 1, wherein the harness is formed from a wire, the wire having a diameter of 0.0075 in. to 0.008 in.

11. The implantable fixation device of claim 1, wherein the biasing element is a leaf spring having a concave surface, a convex surface, a thickness defined between the concave surface and convex surface, and a slot extending through the concave and convex surfaces, the stud extending through the slot, and the thickness of the biasing element being 0.0064 in.

12. The implantable fixation device of claim 11, wherein the biasing element defines a width, and the slot of the biasing element defines a slot width, the width of the biasing element being 0.060 in. to 0.062 in., and the slot width being 0.029 in. to 0.031 in.

13. The implantable fixation device of claim 1, wherein the actuation mechanism includes first and second legs respectively pivotably connected to the first and second fixation elements, and a base pivotably connected to the first and second legs, the stud being connected to and extending from the base.

14. The implantable fixation device of claim 1, wherein the lock further includes a housing, the stud extending into the housing, and the biasing element, wedging element, and single foot being disposed within the housing.

15. The implantable fixation device of claim 14, wherein the housing includes a finger projecting inwardly therefrom and disposed at a first side of stud, and the single foot of the harness is disposed at a second side of the stud opposite of the first side.

16. The implantable fixation device of claim 15, wherein the wedging element has a first end disposed adjacent to the finger and a second end disposed adjacent to the single foot.

17. The implantable fixation device of claim 14, further comprising a coupling member connected to the housing and being configured to releasably connect to a shaft of a delivery system.

18. The implantable fixation device of claim 1, further comprising first and second gripping elements respectively disposed opposite the first and second fixation elements and being moveable relative thereto for capturing tissue therebetween.

19. The implantable fixation device of claim 18, wherein, when tissue is captured between the first and second fixation elements and respective first and second gripping elements, the first and second fixation elements and the lock define a threshold locking angle, the threshold locking angle being 30 degrees or less.

20. An interventional system for fixating tissue comprising:
a delivery system comprising:
a delivery catheter handle having a lock control assembly,
a delivery catheter having a first end coupled to the delivery catheter handle and a second end remote from the first end, and
a lock line extending from the lock control assembly and through the delivery catheter; and
a fixation device releasably coupled to the second end of the delivery catheter, the fixation device comprising:
a first fixation element and a second fixation element, each of the first and second fixation elements having a first end, a free end opposite the first end, and an engagement surface therebetween for engaging tissue, the free ends being moveable between a first position and a second position,
an actuation mechanism coupled to the first and second fixation elements, the actuation mechanism having a stud configured to move in an axial direction, wherein movement of the stud in the axial direction moves the first and second fixation elements between the first and second positions, and
a lock comprising:
a wedging element having a first surface and an opposing second surface, the wedging element being at least partially disposed about the stud and being moveable between a first position in which the wedging element engages the stud to arrest its movement in the axial direction, and a second position in which the wedging element is disengaged from the stud allowing the stud to move in the axial direction,
a biasing element engaging the first surface of the wedging element and being configured to bias the wedging element toward the first position thereof, and
a single-sided harness having a first end portion and a second end portion, the first end portion defining an opening configured to receive the lock line, and the second end portion having a foot, the foot being moveable to engage the second surface of the wedging element and move the wedging element against the bias of the biasing element from the first position to the second position of the wedging element,
wherein the second end of the delivery catheter defines a longitudinal axis, the lock line extends through the opening of the first end portion of the single-sided harness at a first side of the longitudinal axis, and the foot of the single-sided harness is disposed at an opposite second side of the longitudinal axis.
